(19) United States
(12) Patent Application Publication
(10) Pub. No.: US 2016/0045381 A1
(43) Pub. Date: Feb. 18, 2016

(54) METHOD AND SYSTEM FOR MEDICAL KITS FOR TACTICAL DEPLOYMENT

(71) Applicant: Tribalco, LLC, Bethesda, MD (US)

(72) Inventor: David Spence, Tampa, FL (US)

(73) Assignee: TRIBALCO, LLC, Bethesda, MD (US)

(21) Appl. No.: 14/823,362

(22) Filed: Aug. 11, 2015

Related U.S. Application Data
(60) Provisional application No. 62/036,938, filed on Aug. 13, 2014.

(51) Int. Cl. A61F 17/00

(52) U.S. Cl. CPC A61F 17/00

(57) ABSTRACT

Methods and systems are provided that decrease the time and distance from point of wounding to definitive medical care through integrated and specialized soldier survival medical kits and modules. These modules are mission-ready and usable out of the box, easily mission-configured and deployed by tactical operators to any operational location to ensure life saving measures can be rapidly and effectively executed if the need arises. They avoid unnecessary assembly and may involve no or minimal assembly. They make medical care items and supplies easily accessible, while minimizing encumbrances. They include the right equipment positioned in the right places for accessibility, and increase efficiency in use and shipment, and the time savings may save lives. Each module is designed to enable the operator to get the most out of the equipment on hand. Items are specifically chosen and packed in a way that is most efficient, easily accessible, and easily packed to reduce time and effort of use. Furthermore, operators do not need to individually determine what supplies are needed. They may include an extraction kit, a mobility kit, a transportation kit and a sustainment kit.

### RELATED APPLICATION

The present application claims priority to U.S. Provisional Application No. 62/036,938, entitled “Method and System for Medical Kits for Tactical Deployment, filed Aug. 13, 2014, which is incorporated by reference herein.

### FIELD OF THE INVENTION

This generally relates to medical kits, and more particularly to medical and survival kits for deployment for military situations and/or to military personnel.

### BACKGROUND

Operational teams, in particular military teams such as special warfare teams, are often deployed on short notice. Both the transition from inside a country to an overseas operational area and mission deployments are rapid operations that occur in short amounts of time. Conventionally, medical specialists, as key members of tactical teams, prepare their own soldier survival medical kits. Without a defined standard, the sourcing, types and packaging of casualty care supplies and capabilities is typically done in an ad-hoc manner, often by the individual medical member. As a result, mission teams often do not have the optimal capabilities needed to save lives.

Conventional systems may be packed and unpacked individually and then set up for use. These conventional medical supplies are often not mission-ready and ready to use out of the box. They require manual assembly by the operator, and operators often have their own styles and methods of assembly. The need for assembly by individual members for typical medical tasks is often a waste of time and effort, as well as being potentially hazardous to patients that need rapid care. Also, due to ad-hoc compilation, these medical supplies are often not readily usable interchangeably by other members of the team.

Medical supplies and individually made kits may not be packed in a sturdy manner that can withstand elements and impact, or be able to be carried efficiently or attached to vehicles. They may also be inefficiently packed, taking up more space than necessary, while not minimizing weight. They may also not contain useful items, or have items that are not as easily accessible as possible.

As such, there is a desire to avoid these and other related problems.

### SUMMARY

A set of mission-ready tactical medical kits for military personnel is provided, comprising an extraction medical kit comprising first equipment configured to assist a user in extracting a patient at a point of injury, and a mobility medical kit including second equipment configured to assist the user in moving the patient from a point of injury. The set also comprises a transportation medical kit including third equipment configured to assist the user in treating and monitoring the patient during patient transport, and a sustainment kit including fourth equipment configured to assist the user in sustaining a patient for up to three days, the fourth equipment including a pack system including two or more sustainment kit panels configured to attach pouches of medical equipment in the pack system. The extraction, mobility, transportation and sustainment medical kits are usable by the user without further assembly.

A set of mission-ready tactical medical kits for military personnel is provided, comprising an extraction medical kit comprising first equipment configured to assist a user in extracting a patient at a point of injury, and a mobility medical kit including second equipment configured to assist the user in moving the patient from a point of injury. The set also comprises a transportation medical kit including third equipment configured to assist the user in treating and monitoring the patient during patient transport, and a sustainment kit including fourth equipment configured to assist the user in sustaining a patient for up to three days. The extraction, mobility, transportation and sustainment medical kits comprise Rotomold transit cases for storage of the first, second, third and fourth equipment.

A method is provided for providing a set of mission-ready tactical medical kits for military personnel, comprising receiving equipment to assemble medical kits, and assembling, from the received equipment, an extraction medical kit comprising first equipment configured to assist a user in extracting a patient at a point of injury. The method further comprises assembling, from the received equipment, a mobility medical kit including second equipment configured to assist the user in moving the patient from a point of injury, and assembling, from the received equipment, a transportation medical kit including third equipment configured to assist the user in treating and monitoring the patient during patient transport. The method also comprises assembling, from the received equipment, a sustainment kit including fourth equipment configured to assist the user in sustaining a patient for up to three days, the fourth equipment including a pack system including two or more sustainment kit panels configured to attach pouches of medical equipment in the pack system. The extraction, mobility, transportation and sustainment medical kits are usable by the user without further assembly.

A method is provided for providing a set of mission-ready tactical medical kits for military personnel, comprising receiving equipment to assemble medical kits, and assembling, from the received equipment, an extraction medical kit comprising first equipment configured to assist a user in extracting a patient at a point of injury. The method further comprises assembling, from the received equipment, a mobility medical kit including second equipment configured to assist the user in moving the patient from a point of injury, and assembling, from the received equipment, a transportation medical kit including third equipment configured to assist the user in treating and monitoring the patient during patient transport. The method also comprises assembling, from the received equipment a sustainment kit including fourth equipment configured to assist the user in sustaining a patient for up to three days. The extraction, mobility, transportation and sustainment medical kits comprise Rotomold transit cases for storage of the first, second, third and fourth equipment.

### BRIEF DESCRIPTION OF THE DRAWINGS

FIG. 1 illustrates a complete set of the medical kit, the transit cases, and the modules in accordance with the present invention.

FIG. 2A illustrates an exemplary extraction kit in accordance with the present invention.

FIG. 2B illustrates an exemplary mobility kit in accordance with the present invention.

FIG. 2C illustrates an exemplary transportation kit in accordance with the present invention.

FIG. 2D illustrates an exemplary sustainment kit in accordance with the present invention.

FIG. 3A illustrates a pallet assembly in accordance with the present invention.

FIG. 3B illustrates a pallet sub-assembly in accordance with the present invention.

FIG. 3C illustrates a pallet sub-assembly in accordance with the present invention.

FIG. 4A illustrates an exemplary E-1 and E-2 (E-1/2) transit case of the extraction kit (E) and its contents.

FIG. 4B illustrates an exemplary E-1 Pack system of the E-1/2 Transit Case.

FIG. 4C depicts an exemplary E-1 Top Pouch of the E-1/2 Transit Case.

FIG. 4D shows an exemplary E-1 Lateral Bag Set of the E-1/2 Transit Case.

FIG. 4E illustrates an exemplary E-1 Lateral Bag Set of the E-1/2 Transit Case.

FIG. 4F illustrates the pack contents of an exemplary E-1 Pack System.

FIG. 4G illustrates the pack contents of an exemplary E-1 Pack System.

FIG. 4H shows an exemplary E-2 Pack System of the E-1/2 Transit Case.

FIG. 4I illustrates an exemplary E-2 Top Pouch of the E-1/2 Transit Case.

FIG. 4J illustrates an exemplary E-2 Bottom pouch of the E-1/2 transit case.

FIG. 4K illustrates the pack contents of an exemplary E-2 Pack system

FIG. 4L illustrates the pack contents of an exemplary E-2 Pack System.

FIG. 4M illustrates an exemplary E-2 High-angle recovery bag.

FIG. 4N illustrates an exemplary E-2 High-angle recovery bag.

FIG. 4O illustrates an exemplary E-2 Bottom storage of the E-1/2 Transit case.

FIG. 5A illustrates the exemplary E-3 Transit case and its contents.

FIG. 5B illustrates the exemplary LE Pack System of the E-3 transit case.

FIG. 5C illustrates the exemplary LE (Light Extrication) Pack of the E-3 transit case.

FIG. 5D illustrates the exemplary LE Pouches of the E-3 transit case.

FIG. 5E illustrates the exemplary LE Pack Contents of the E-3 transit case.

FIG. 5F illustrates the exemplary LL Pack System of the E-3 Transit Kit.

FIG. 5G illustrates the exemplary LL Pouches of the E-3 Transit Case.

FIG. 5H illustrates the exemplary LL Pack Contents of the E-3 Transit Case.

FIG. 5I illustrates the exemplary Bottom Storage of the E-3 Transit Case.

FIG. 6A illustrates the exemplary case contents of the M-1 Transit Case of the mobility kit.

FIG. 6B illustrates the exemplary M-2 Pack System of the M-1 Transit Case.

FIG. 6C illustrates the exemplary M-2 Pack System of the M-1 Transit Case.

FIG. 6D illustrates the exemplary M-2 Pouch Components of the M-1 Transit Case.

FIG. 6E illustrates the exemplary M-2 IFAK Pouch of the M-1 Transit Case.

FIG. 6F illustrates the exemplary M-2 IFAK Pouch of the M-1 Transit Case.

FIG. 6G illustrates the exemplary M-2 Strap & Hypothermia Pouches of the M-1 Transit Case.

FIG. 6H illustrates the exemplary M-2 Mass Hem Pouch of the M-1 Transit Case.

FIG. 6I illustrates the exemplary M-2 Circulation Pouch of the M-1 Transit Case.

FIG. 6J illustrates the exemplary M-2 Circulation Pouch of the M-1 Transit Case.

FIG. 7 illustrates the exemplary Med Storage Chest of the S-1 Transit Case of the sustainment kit.

FIG. 8A illustrates the exemplary case contents of the S-1 Transit Case of the sustainment kit.

FIG. 8B depicts an exemplary pack system of the S-1 Transit Case.

FIG. 8C illustrates the exemplary Pack System of the S-1 Transit Case.

FIG. 8D illustrates the exemplary top pouch of the S-1 Transit Case.

FIG. 8E illustrates the exemplary bottom pouch of the S-1 Transit Case.

FIG. 8F illustrates an exemplary IFAK Pouch of the S-1 Transit Case.

FIG. 8G illustrates an exemplary IFAK Pouch of the S-1 Transit Case.

FIG. 8H depicts an exemplary Airway Pouch of the S-1 Transit Case.

FIG. 8I illustrates the exemplary Airway Pouch of the S-1 Transit Case.

FIG. 8J illustrates an exemplary Respiratory Pouch of the S-1 Transit Case.

FIG. 8K illustrates an exemplary Circulation Pouch of the S-1 Transit Case.

FIG. 8L illustrates the exemplary Circulation Pouch of the S-1 Transit Case.

FIG. 8M illustrates the exemplary WWTK Pouch of the S-1 Transit Case.

FIG. 8N illustrates an exemplary Hypothermia Pouch of the S-1 Transit Case.

FIG. 8O illustrates the exemplary Bottom Storage of the S-1 Transit Case.

FIG. 9A illustrates exemplary case contents of the S-2 Transit Case of the sustainment kit.

FIG. 9B illustrates the exemplary Pack System of the S-2 Transit Case.

FIG. 9C illustrates the exemplary Pack System of the S-2 Transit Case.

FIG. 9D illustrates the exemplary Lower Pouch of the S-2 Transit Case.

FIG. 9E illustrates the exemplary IFAK Pouch of the S-2 Transit Case.

FIG. 9F illustrates the exemplary IFAK Pouch of S-2 Transit Case.

FIG. 9G illustrates the exemplary Airway Pouch of the S-2 Transit Case.

FIG. 9H illustrates the exemplary S-2 Transit Case: Airway Pouch (2 of 2).

FIG. 9I illustrates the exemplary Respiratory Pouch of the S-2 Transit Case.

FIG. 9J illustrates the exemplary Circulation Pouch of the S-2 Transit Case.

FIG. 9K illustrates the exemplary Circulation Pouch of the S-2 Transit Case.

FIG. 9L illustrates the exemplary WWTK Pouch of the S-2 Transit Case.

FIG. 9M illustrates the exemplary Hypothermia Pouch of the S-2 Transit Case.

FIG. 9N illustrates the exemplary Trauma Kit of the S-2 Transit Case.

FIG. 9O illustrates the exemplary Trauma Kit of the S-2 Transit Case.

FIG. 9P illustrates the exemplary DA Medical Bag of the S-2 Transit Case.

FIG. 9Q illustrates the exemplary DA Medical Bag of the S-2 Transit Case.

FIG. 9R illustrates the exemplary Med Bag Organizer Panel of the S-2 Transit Case.

FIG. 9S illustrates the exemplary Med Bag Airway Pouch of the S-2 Transit Case.

FIG. 9T illustrates the exemplary Med Bag Respiratory Pouch of the S-2 Transit Case.

FIG. 9U illustrates the exemplary DA Medical Bag Circulation 1 Pouch S-2 Transit Case.

FIG. 9V illustrates the exemplary DA Medical Bag Circulation 2 Pouch of the S-2 Transit Case.

FIG. 9W illustrates the exemplary DA Medical Bag Multi Pouch of the S-2 Transit Case.

FIG. 9X illustrates the exemplary bottom portion of the S-2 Transit Case.

FIG. 10A illustrates the exemplary Case Contents of the T-1 Transit Case.

FIG. 10B illustrates the exemplary T-1 and T-2 Case (T-1/2) of the T-1 Transit Case.

FIG. 10C illustrates the exemplary T-1/2 Case of the T-1 Transit Case.

FIG. 10D illustrates the exemplary T-1/2 Case of the T-1 Transit Case.

FIG. 10E illustrates the exemplary T-1/2 Case of the T-1 Transit Case.

FIG. 10F illustrates the exemplary T-1/2 Case of the T-1 Transit Case.

FIG. 10G illustrates the exemplary T-1/2 Case of the T-1 Transit Case.

FIG. 10H illustrates the T-3 and T-4 (T-3/4) Case of the T-1 Transit Case.

FIG. 10I illustrates the T-3/4 Case of the T-1 Transit Case.

FIG. 10J illustrates the T-3/4 Case of the T-1 Transit Case.

FIG. 10K illustrates the T-3/4 Case of the T-1 Transit Case.

FIG. 10L illustrates the T-3/4 Case of the T-1 Transit Case.

FIG. 10M illustrates the exemplary T-3/4 SeQual Oxygen System of the T-1 Transit Case.

FIG. 10N illustrates the exemplary T-5 module of the T-1 Transit Case.

FIG. 10O illustrates the exemplary T-6 module of the T-1 Transit Case.

FIG. 11 is a flowchart that illustrates an exemplary method for providing medical kits in accordance with the present invention.

FIG. 12 illustrates an exemplary method for using medical kits in accordance with the present invention.

### DETAILED DESCRIPTION

Methods and systems in accordance with the present invention decrease the time and distance from point of wounding to definitive medical care through uniquely integrated and specialized soldier survival medical kits and modules. These modules are mission-ready and usable out of the box, easily deployed by tactical operators to any operational location to ensure life saving measures can be rapidly and effectively executed if the need arises. They may be able to be used at a moment's notice without having to be assembled first. They avoid unnecessary assembly and may involve no or minimal assembly. They make medical care items and supplies easily accessible, while minimizing encumbrances. Providing time savings that may save lives, they include the right equipment positioned in the right places for accessibility, and increase efficiency in use and shipment. Each module is designed to enable the operator to get the most out of the equipment on hand. Items are specifically chosen and packed in a way that is most efficient, easily accessible, and easily packed to reduce time and effort of use. Furthermore, operators do not need to individually determine what supplies are needed.

Methods and systems in accordance with the present invention provide a comprehensive medical set usable by military or other personnel that is modular in design, borne from an international standard of training, interoperable across a majority of platforms worldwide and provides the capability of a multinational force to operate together with similar skills, doctrine, and equipment. Method and systems in accordance with the present invention fill mission gaps and enhance, for example, the Advanced Tactical Practitioner's (ATP's) ability to keep a wounded Special Operations Forces (SOF) member alive from the point of wounding to the point of definitive care. The methods and systems may apply to military special forces, any military or even non-military scenarios and personnel. The system enables, for example, United States Special Operations Command (SOCOM) teams to move the most advanced care as far forward as possible. These methods and systems enhance the modularity, accessibility, and kit configurations for the entire system, allowing for the simple, efficient extraction, movement, transportation, and sustainment of the wounded operator. The system also provides maximum accessibility to the most complicated medical equipment to be used.

These methods and systems enhance the abilities of medical specialists to take care of personnel from point-of-wounding, care under fire, tactical field care, Casualty Evacuation (CASEVAC) care stages and finally to definitive care at the Combat Surgical Hospital (CSH). Medical specialists have numerous options to configure their kits to their specific mission requirements using standardized kits and modules. The modular system allows for the customization of pre-packaged equipment, enabling the medic to customize their workspace used in their environment. This may include, for example, adding modular pockets, drawer configurations, night vision labels, etc. This ability to rapidly configure equipment better enables teams to react to time-sensitive targets (TSTs). With the time constraints of post-mission requirements, methods and systems in accordance with the present invention minimize repacking and recharging of equipment because of its specialized configuration and ease of access. Teams no longer have to unpack, but rather simply plug in rechargeable units. Dust covers and the like protect unused gear and decreases routine cleaning and maintenance. Cards providing nomenclature and pictures with packing sets enable team members to inventory a piece of equipment even if it is not their specialty.

These methods and systems are not an off-the-shelf existing system adopted from catalogues or from civilian emergency medical systems. They may be comprised of customized, combat-proven products and organized in a manner to be efficiently focused for each mission. These methods and system overcomes challenges identified by the operators, such as operation in austere environments and improvised patient treatment stations in vehicles or host nation buildings.

These systems offer mission effectiveness, decreasing the time and distance from point of wounding to definitive care through specialized modules. Operators can configure the modules for mounted (on a vehicle) or dismounted use so that the modules can be strategically placed closer to target structures.

These methods and systems provide modularity. The modular transit cases, containers and enclosures protect the valuable contents from the environmental elements such as rain, dust, and dirt. With the survival of the patients at stake, the system is designed for ruggedness and reliability of the containers. Equipment also arrives fully mission-capable, which places a premium on weather and climate protection.

Modularity and shipping containers assist in the performance of the system. For example, the modularity helps in arranging kits on Air Force pallets, for example, which decreases the time taken by teams to palletize equipment.

In one implementation, Loadmaster Rotomold cases are used which may work like building blocks, and there are many possible combinations when stacking groups of Loadmaster Rotomold cases. As an example, two 12-inch high cases can be stacked together alongside one 24-inch high case. A fourth case can be stacked over the tops of all three lower cases. Modular dimensions and stacking features, molded into the top and bottom surfaces of Loadmaster cases, allow these cases to securely interlock when stacked. ECS Composites cases are proven waterproof/dustproof containers that are able to stack in the most efficient way possible. These cases were selected and tested for durability with high altitude drops and other methods to ensure proper field operation.

With respect to durability and impact resistance, for example, the sustainment kit transit cases discussed below may have an additional 2″ of high density foam to allow for high-impact resistance within the cases to meet A Series Cargo Delivery System (CDS) air delivery requirements.

Methods and systems in accordance with the present invention may provide enhanced maintainability. The cases (for example, ECS cases) are purposefully designed to be water- and dust-proof With lids closed, cleaning can be done with water hoses and brushes. Loadmaster Rotomold transit cases comply with applicable performance requirements of the following commonly used standards and specifications, for example: MIL-P-116, MIL-STD-130, MIL-T-28800F, MIL-C-4150J, MIL-STD-454, ATA-300, MIL-T-21200, MIL-T-4734, MIL-STD-648C, MIL-STD-1472, and FED TEST METHOD STD 101.

In the critical few minutes it takes to stabilize a patient, there is little time to be spent finding and unpacking boxes and setting up equipment. Method and systems in accordance with the present invention provide an efficient and effective way to reduce the set-up time for transition from tactical field care to CASEVAC (Casualty Evacuation) care. In today's current environment, soldiers are using common aircraft or vehicles to perform MEDEVAC missions. Conventional CASEVAC platforms are not configured for patient treatment.

Furthermore, the system may include small, light-weight, interlocking carbon-fiber cases for the components integrated into the transportation systems. These carbon fiber cases are designed to fit in available space in aircraft, watercraft or ground vehicles. Also, they can be integrated placed in back pack systems for a man-packable option. Important components may be packaged together, and the set-up time to utilize these components is negligible. This allows SOF ATPs to administer advanced care as fast as possible and as far forward as possible.

With these methods and systems, task forces may patient treatment capabilities on both missions or in instances where SOCOM units provide QRF to each other. The transportation kits, for example, are adaptable to the skill level of the medical providers on mission and enable the ATP, SOF PA's and surgeons to effect care in forward-deployed locations. Finally, these systems may turn a vehicle or room in a building from a collection point to an advanced care point.

Conventional systems are typically packed, unpacked individually and then set up for use. Through a set of fasteners on carbon fiber containers, these custom containers can be mounted vertically and horizontally so that they can fit in the best places to allow a SOF medic to operate them. By being in the right places on vehicles and aircraft, those initial moments of loading a patient can be spent treating the patient rather than setting up equipment.

Furthermore, stabilization of the patient can be accomplished without setting up a stand because the configuration of the cases and boxes provides it already. Lids have quick detachable faces that reveal monitors, cables, and tubing, and, in one implementation, all are ready to be used and attached to a patient. Foam inserts provide structure and organization to key components. Additionally, the foam inserts are designed to pull out so that space behind devices can be available for additional storage of batteries, accessory power cords, tubing, etc.

Finally, transportation module carbon fiber carrying cases interlock with shared adaptors integrated a backpack system. This interlocking system maximizes stability, minimizes strap retainers, and makes these critical items available during dismounted operations.

In addition, a labeling product is provided to clearly mark kit components: a glow thread labeling system enables the SOF ATP to clearly identify kit items during the day or, more importantly, at night, utilizing night vision equipment. Pockets are packed and labeled according to the MARCH protocol (Massive Hemorrhage, Airway, Respiratory, Circulation, Head Injury/Hypothermia), a method to treat patients. This enables easy recognition during stressful combat care. Additionally, to allow for ATP customization, each label may be Velcro-backed and may be placed within the kits to meet the operator's individual configuration requirements.

The medical kit set in accordance with the present invention facilitates the rescue, recovery, and evacuation of casualties, and stabilizes and sustains casualties in case of a delayed or difficult CASEVAC and is capable of adapting to military and non-military modes of transportation. The equipment in the set is also man-packable. The man-portable packaging comprises man-packable packaging and/or ruggedized, air-worthy containers suitable to organize transport and protect the equipment/supplies stored within.

Additionally, the system provides simplicity and accessibility. The medical kit set's packaging is simple in design and permits easy access to the equipment/supplies contained within. In one implementation, the kit set's packaging is no more than one-zipper deep to access the main contents of any bag. Its packaging may include basic labels for each compartment and allow the equipment and supplies to be accessible by medics wearing gloves. The set may have universal connectors, straps, and other attachments to safely and quickly secure the individual modules to SOF standard and non-standard mobility platforms.

In one implementation, dual sustainment kit panels as described below are used as opposed to a single sustainment panel. These sustainment panels include various medical components described below, and the use of dual sustainment panels reduces the size and length of the sustainment panel and pack size, while providing additional storage of medical related components. A single longer sustainment panel increases the size and encumbrance of the pack, while potentially reducing carrying capacity. Dual panels (versus one) allow for additional customization when working in confined spaces.

The system combines access capabilities, mobility, and critical care components to support SOFs, for example, in far forward and austere environments where definitive medical support is not available. They offer advanced system modularity and accessibility. These features enhance decentralized teams' ability to self-recover by enabling SOFs to move advanced care as far forward as possible. They aid the advanced tactical practitioner's ability to keep a wounded SOF member alive from the point of wounding to the point of definitive care.

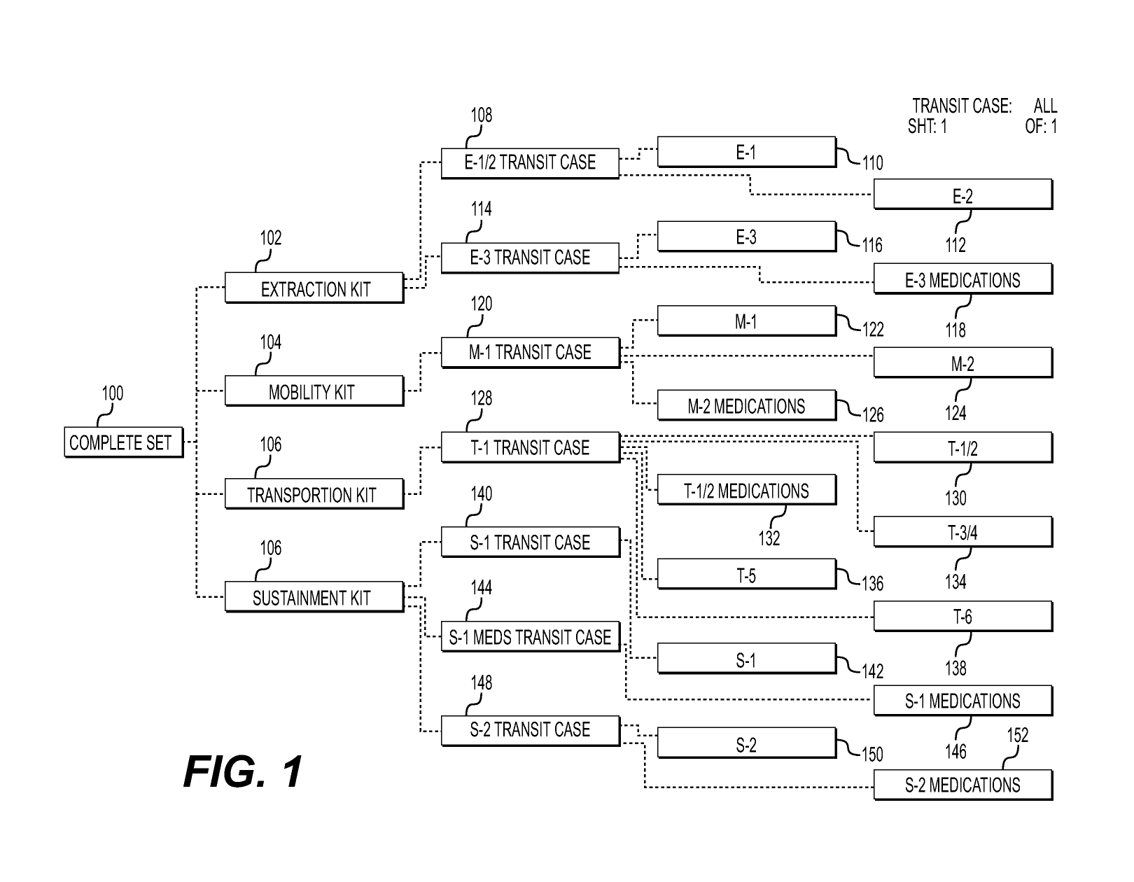

FIG. 1 illustrates an exemplary complete set of medical kits and included modules in accordance with methods and systems consistent with the present invention. The set 100 is broken down into four kits (Extraction 102, Mobility 104, Transport 106 and Sustainment 108) that each provide a specific capability and can be procured and/or used as a whole or individually.

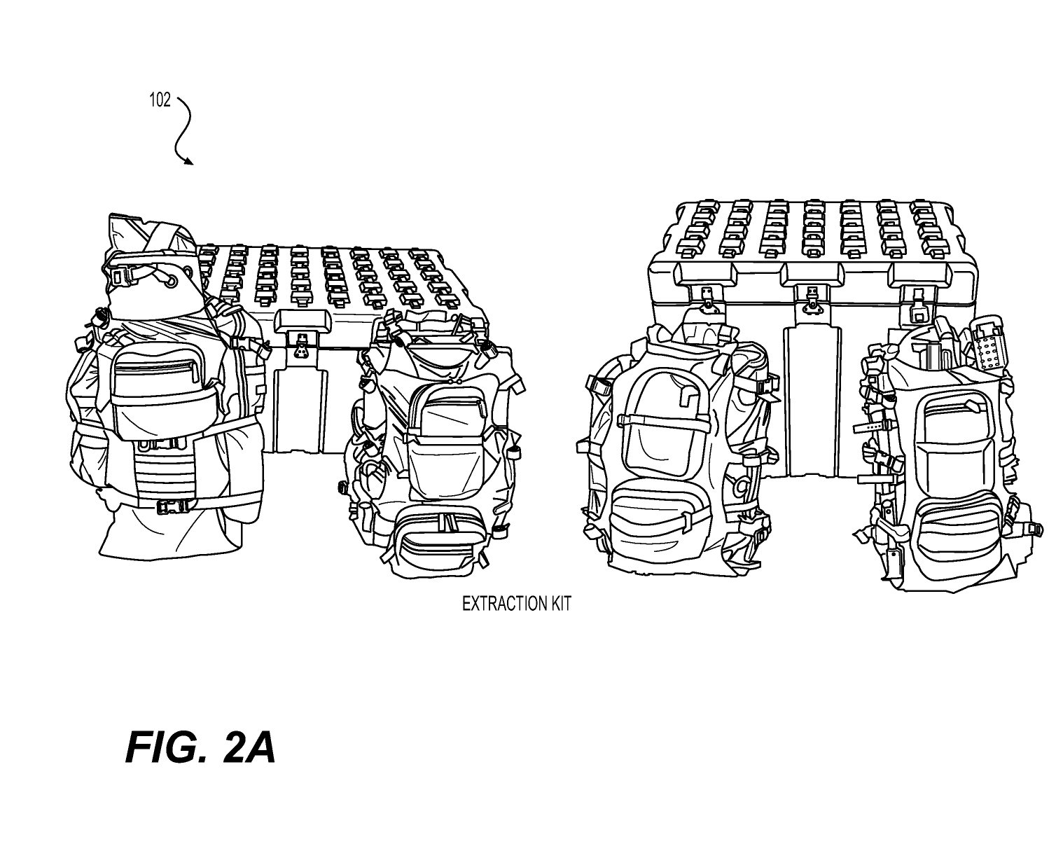

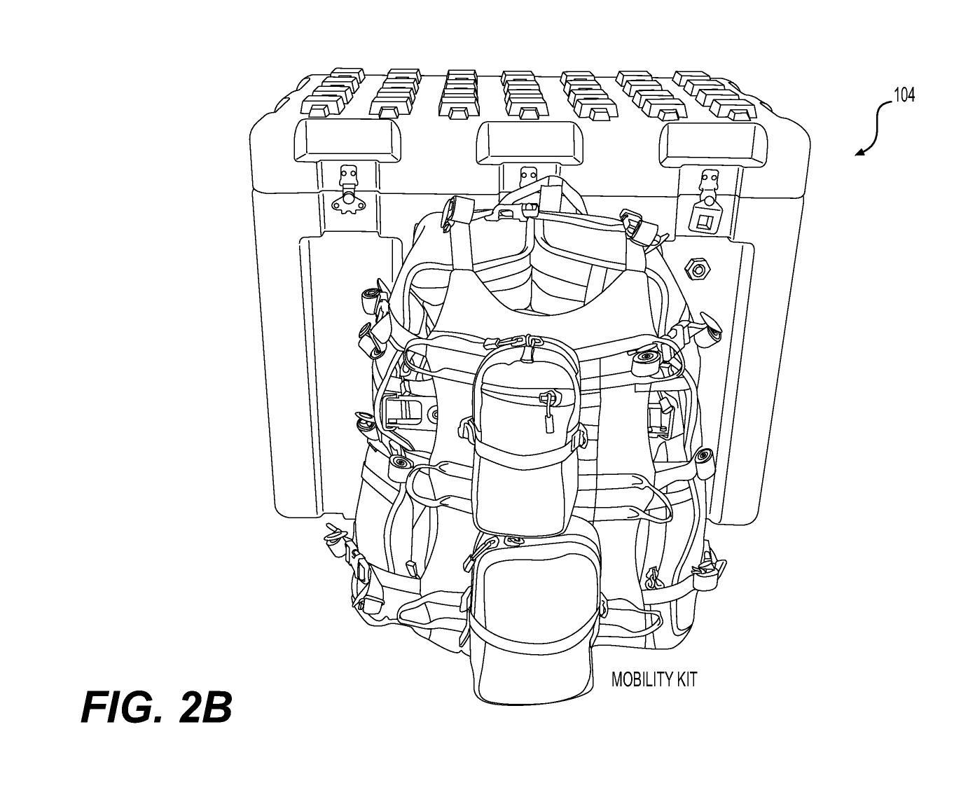

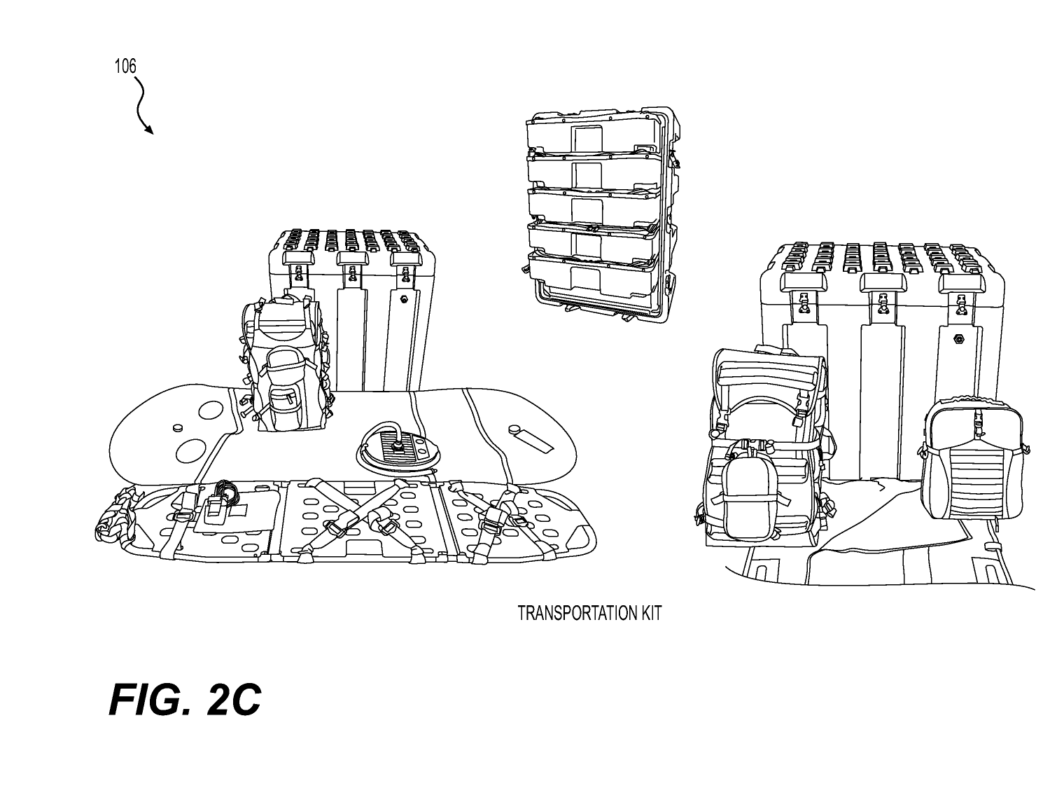

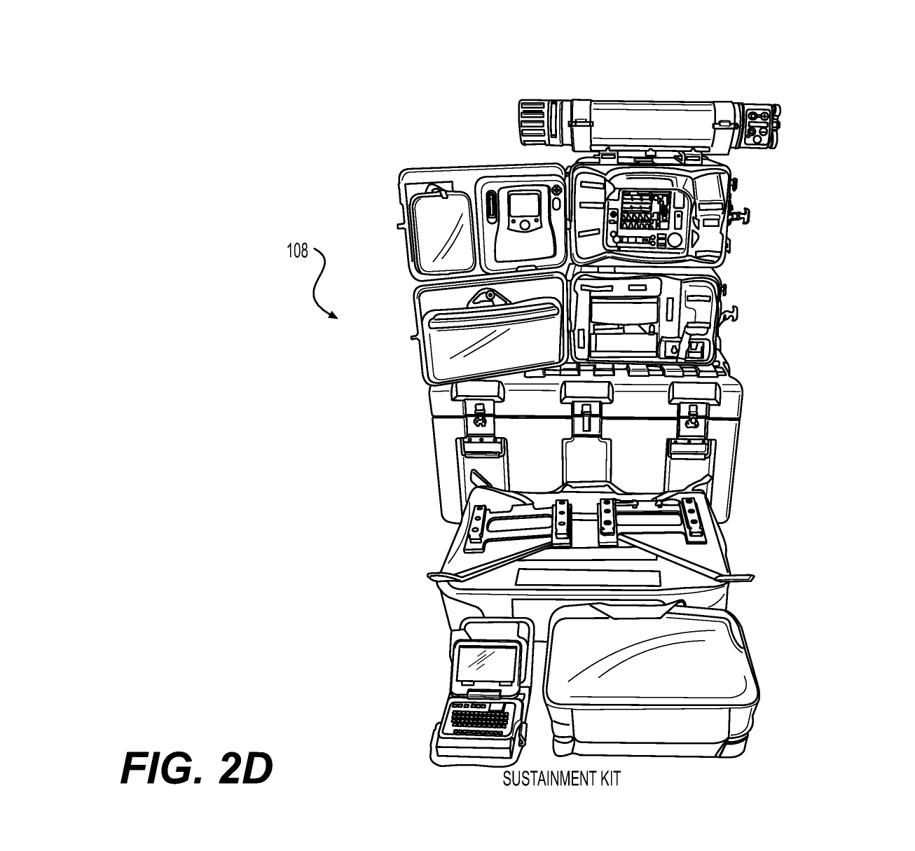

FIG. 2A also shows the extraction kit 102, shown on FIG. 1. The extraction kit 102 provides the ability to access and extricate the patient from various locations. It ensures personnel have the equipment to extricate casualties, e.g., wounded patients, from various terrain, including water obstacles, high angle conditions, urban conditions and in wreckage, during day or night. It includes the E-1/2 transit case 108 for the E1 and E2 modules (110 and 112) and the E-3 transit case 114 for the E-3 module 116 and E-3 medications 118. The E-1 module 108 has the capability to extricate casualties from terrain including water during day and night, and to package a casualty with a spinal injury. The E2 module 110 provides the capability to safely access and recover casualties from high angle terrain and urban obstacles using rope and rigging equipment. The E-3 module 112 provides the capability to pry and cut wreckage to facilitate rapid extrication, and lift and move large objects, e.g., vehicles, wreckage, building debris, etc. enough to remove trapped casualties.

FIG. 2B also shows the mobility kit 104, shown on FIG. 1. The mobility kit 104 provides the ability to move the patient from the point of injury to cover and casualty evacuation care. It generally provides the capability to move the patient on a rigid litter (e.g., portable stretcher) to medical treatment. It includes the M-1 Transit case 120 which includes the M-1 module 122, M-2 module 124 and M-2 medications 126. The M-1 module 122 provides the capability to secure and safely transported casualty on a rigid litter to medical treatment. The M-2 module 124 provides the capability to attach a rigid litter from the M1 module to, for example, SOF ground or air mobility platforms, day and night landing zone markings, and a casualty marking system to identify basic triage categories. The M-2 medications 126 provide related medical supplies.

FIG. 2C also shows the transportation kit 106, shown on FIG. 1. The transportation kit 106 provides the ability to provide life support measures and monitoring during critical care transport. It includes the T-1 transit case 128 and modules T-1 through T6 (130-138), as well as the T-1/2 medications 132. The T-1 and T-2 modules 130 provide the capability to monitor casualty vital signs, and provide electric cardiac therapy. The T-3 and T-4 modules 134 provide the capability to provide oxygen and mechanical ventilation, and to operate and charge associated pieces of medical equipment. The T-5 module 136 provides the capability to warm casualties with decreased body temperature. The T-6 module 138 provides the capability to mount the medical equipment in modules T1-5 to the rigid litter of the mobility kit 104.

FIG. 2D also shows the sustainment kit 108, shown on FIG. 1. The sustainment kit 106 provides the capability to reconstitute the set internally during isolated deployments where outside support is not readily available and to enable units to sustain 2-5 casualties for up to 72 hours in one implementation. It includes the S-1 and S-2 transit cases (140 and 148), which include the S-1 and S-2 modules (142 and 150) respectively. The sustainment kit 106 also includes the S-1 medications transit case 144, which includes the S-1 medications 152. The S-1 module 142 provides the capability to sustain the control of life-threatening bleeding, prevention of shock, protection from wounds, manage pain and control vomiting for 24 hours, for example. It also provides additional medic kit supplies and patient communicators, as well as other functions and supplies. The S-2 module 150 provides advanced airway care, including surgical airway procedures, and also provides mechanical ventilation as well as additional medic kit supplies.

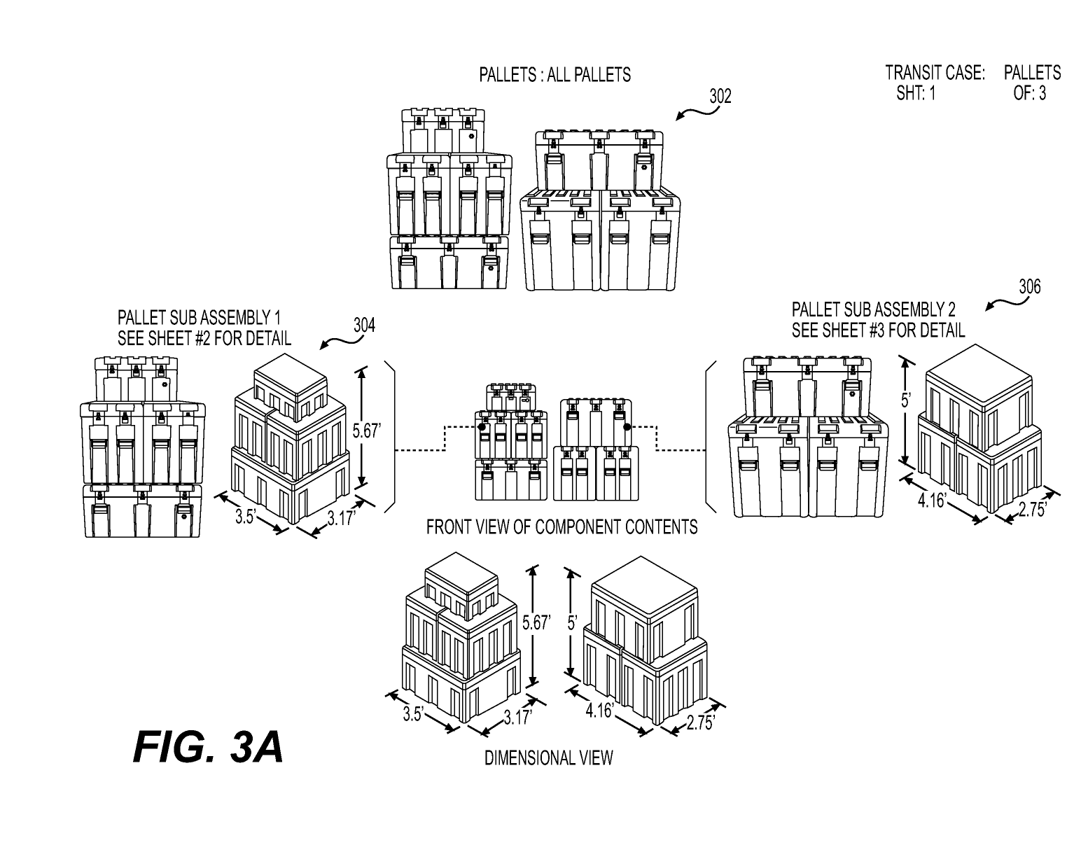

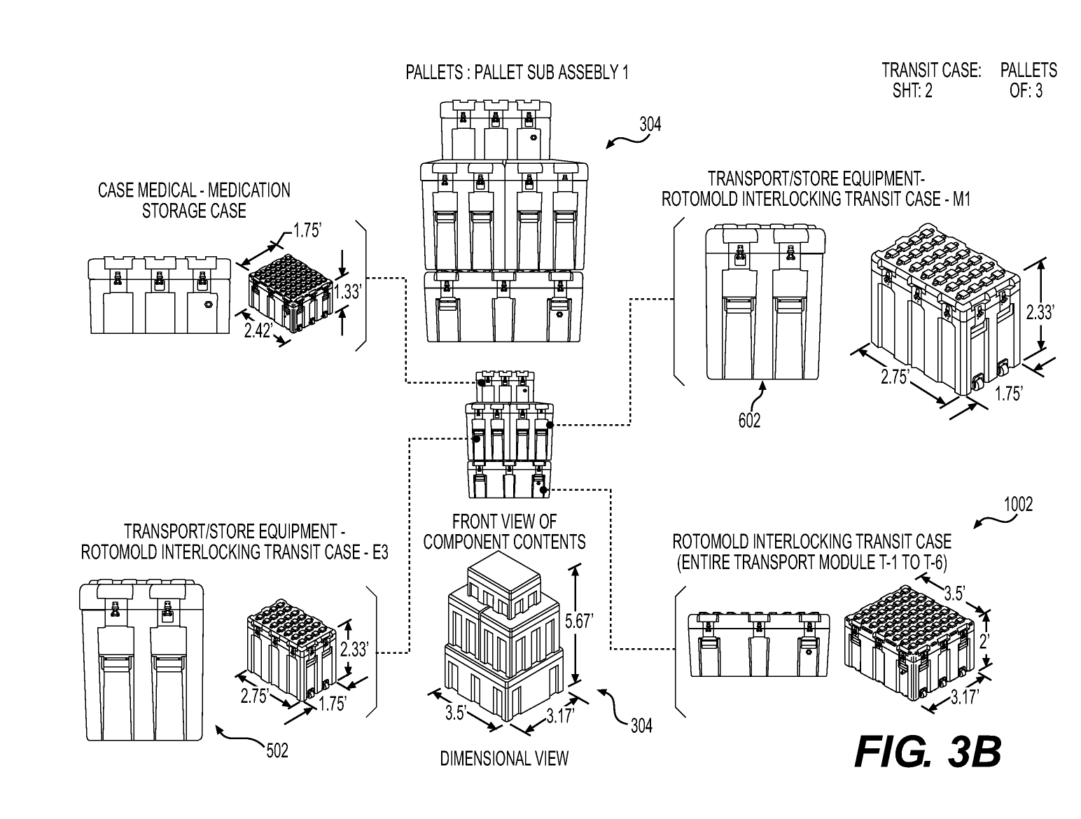

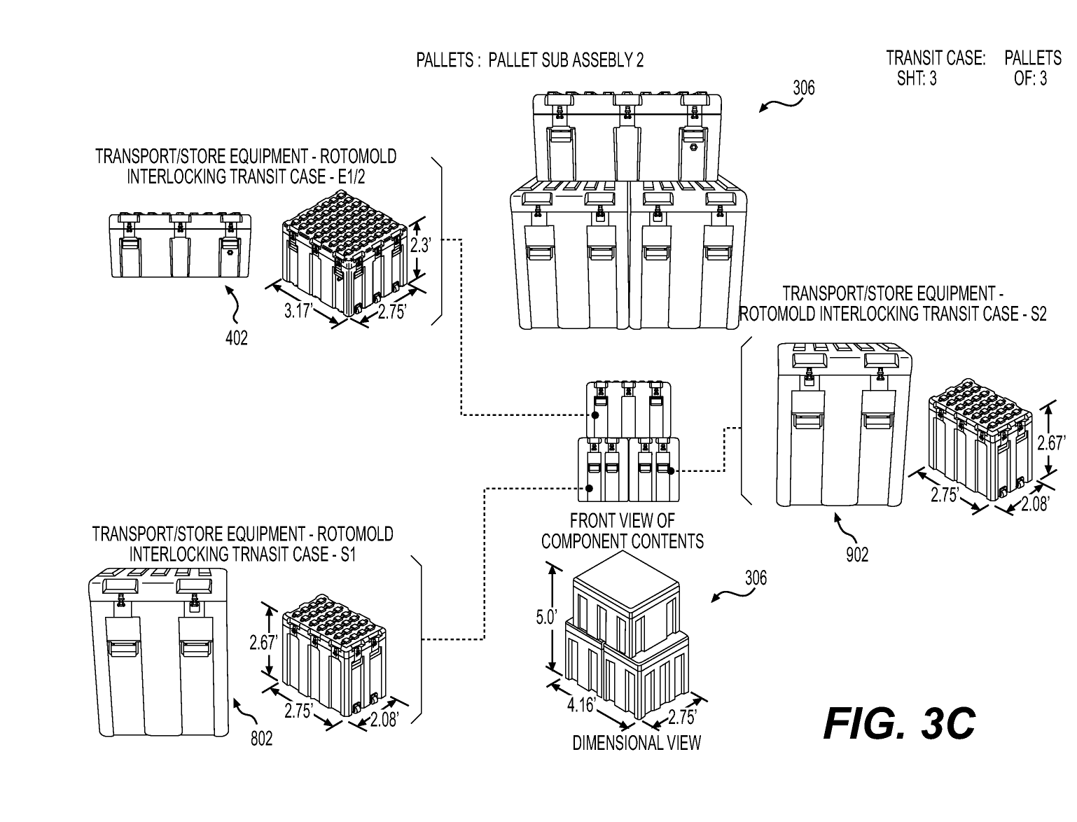

FIG. 3A shows a pallet assembly for assembling the transit cases for transportation, shipment or delivery. As shown, and exemplary configurations for stacking of transit cases is shown in the pallet assembly 302. This pallet assembly includes two pallets subassemblies, 304 and 306. These pallets may be assembled accordingly for increased space efficiency and in order to minimize cube space taken on vehicles, and decreased time to stack modules. Other suitable configurations may also be used.

FIG. 3B shows a first pallet subassembly 304. As shown, the S1 Medication storage case 701 is shown stacked on top of the Rotomold transit cases E3 502 and M1 602, which are side-by-side. These are all stacked on top of the rotor mold interlocking T-1 transit case 1002 discussed below.

FIG. 3C shows a second pallet subassembly 306. As shown, the rotomold transit case E1/2 402 is shown stacked on top of the Rotomold transit cases S1 802 and S2 902, which are side-by-side. These are both stacked on top of the Rotomold interlocking transit cases S1 and S2, which provide the base for this configuration.

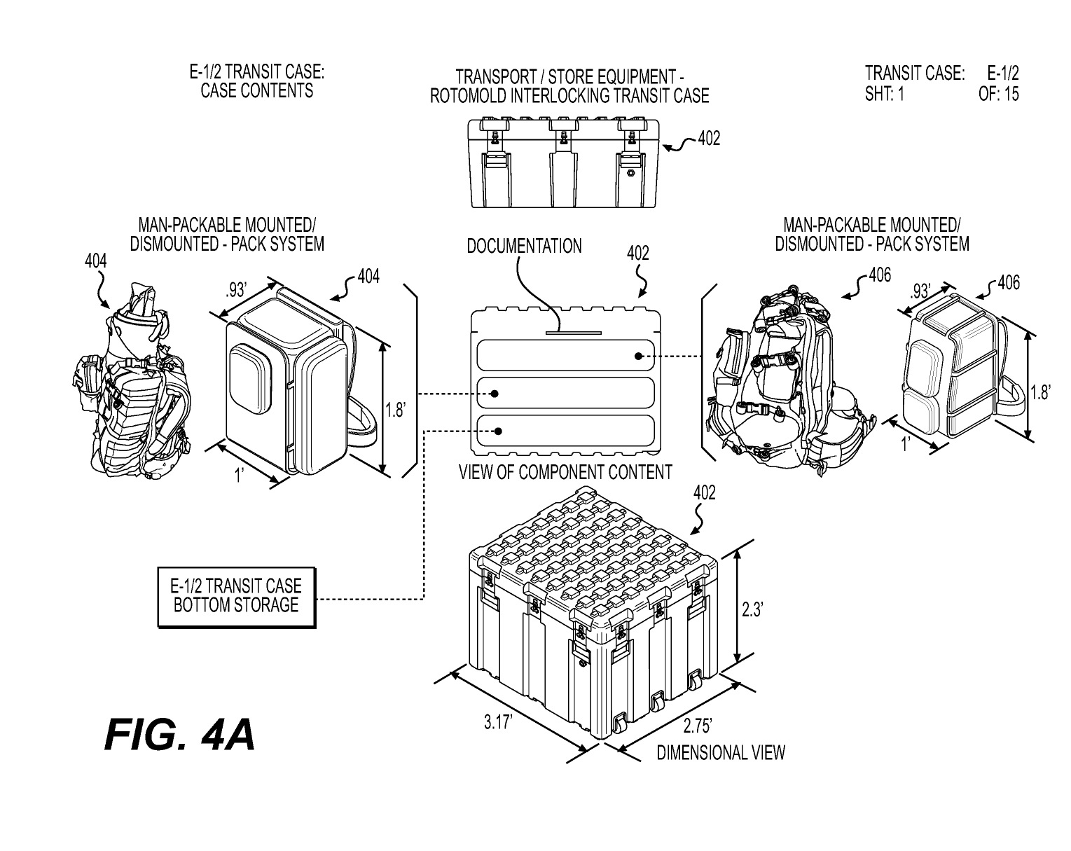

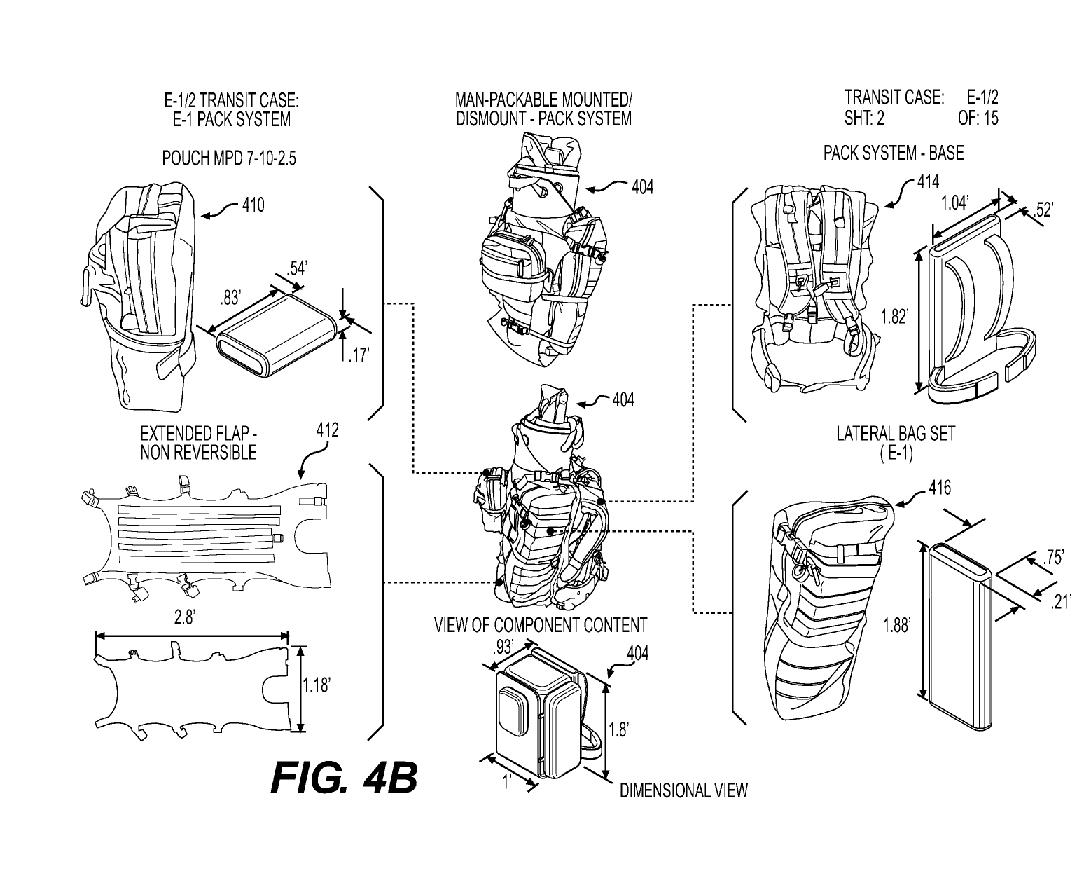

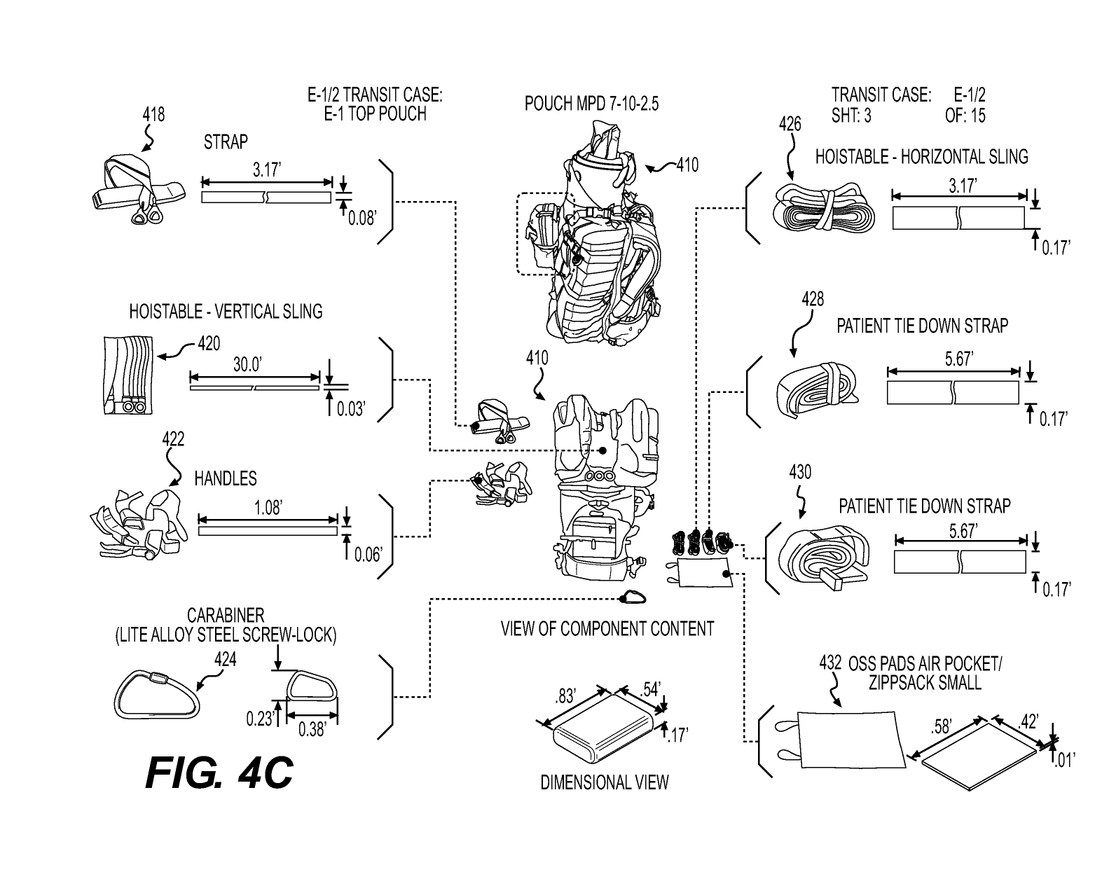

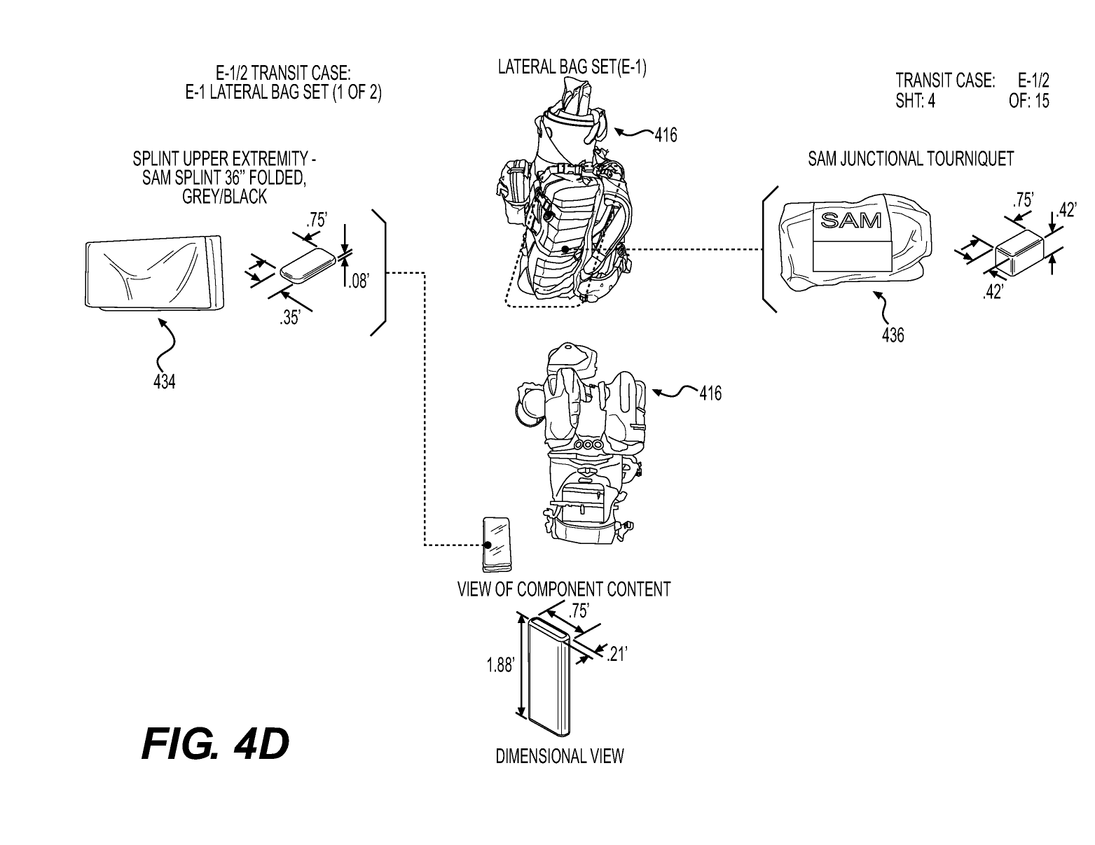

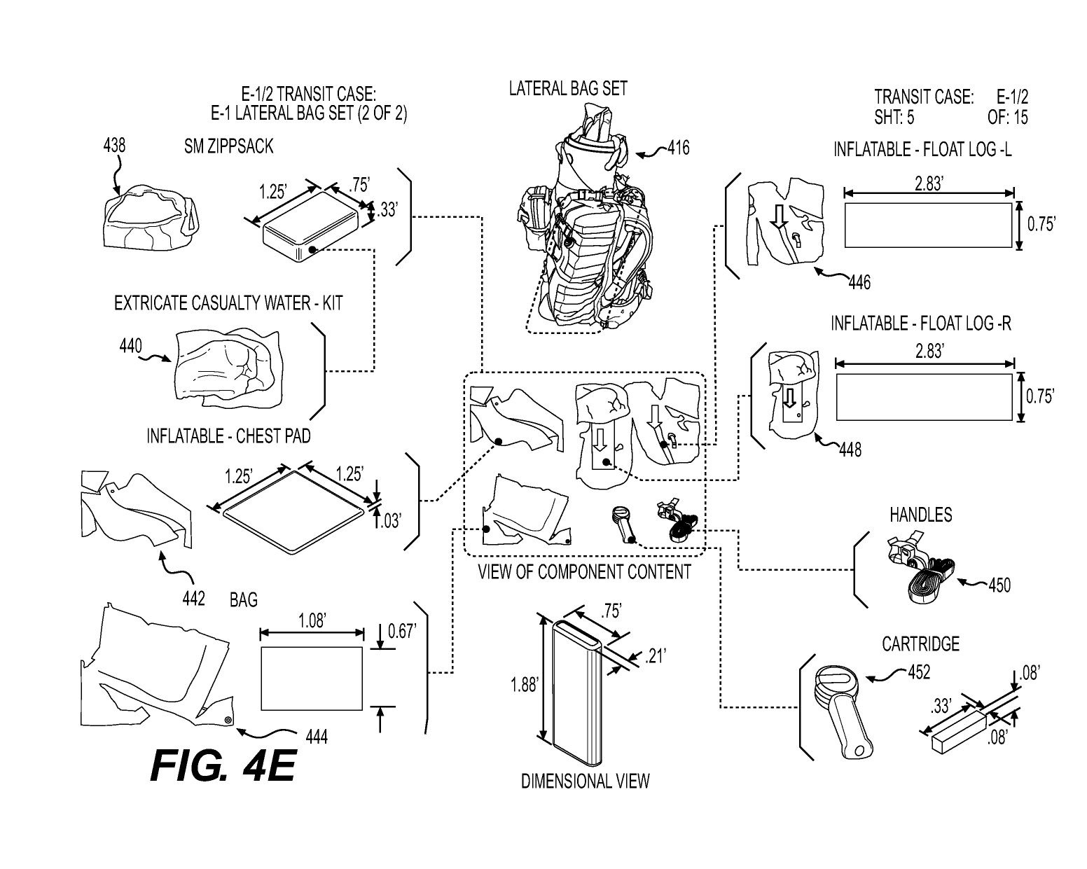

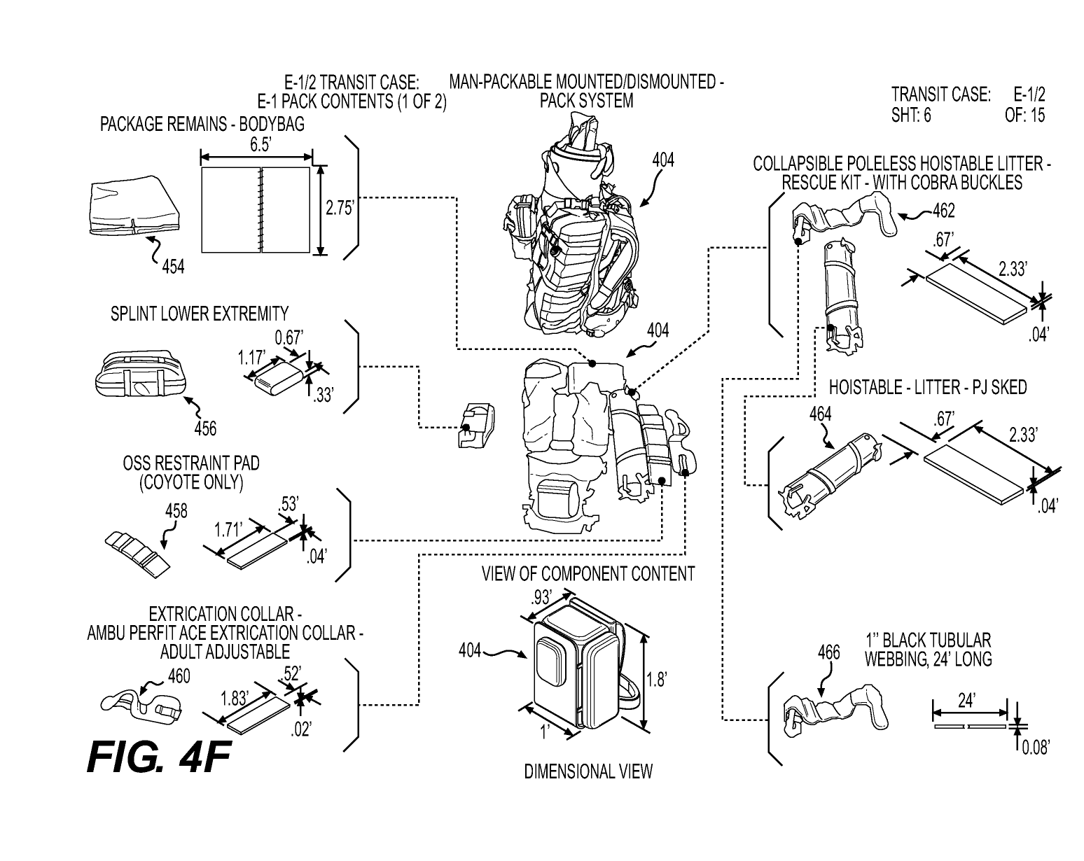

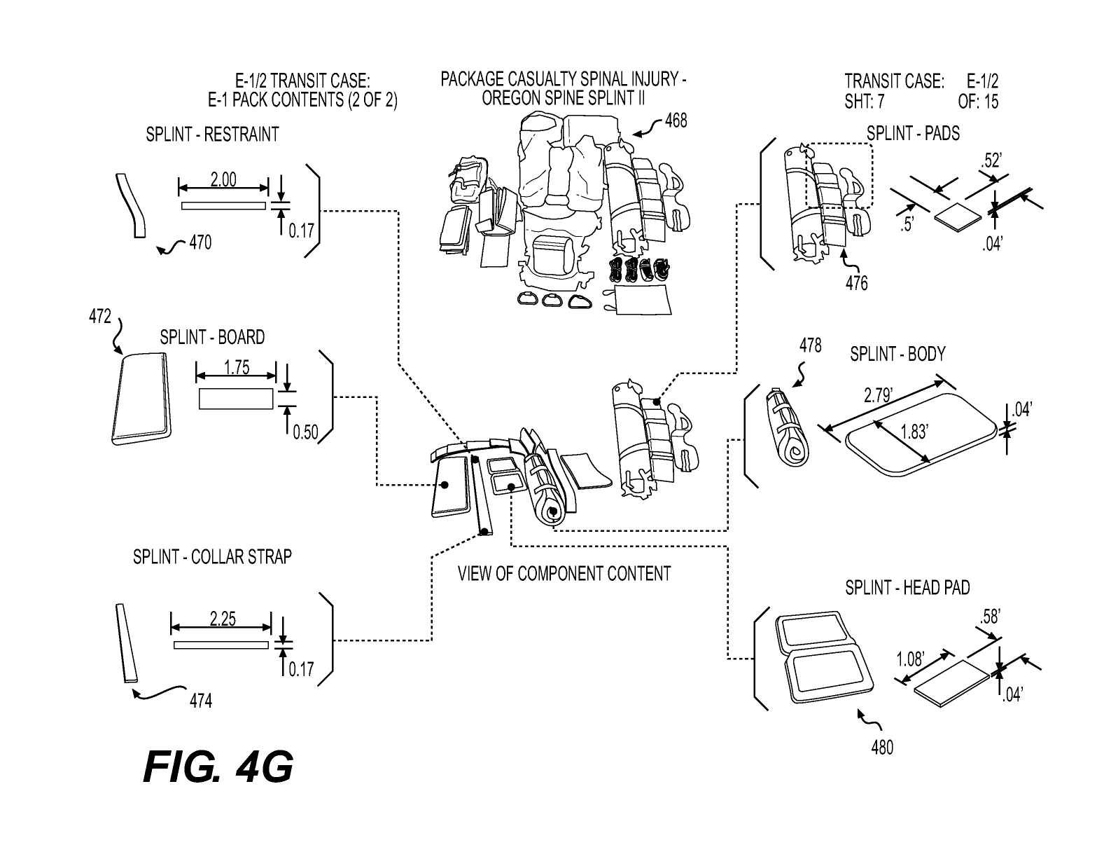

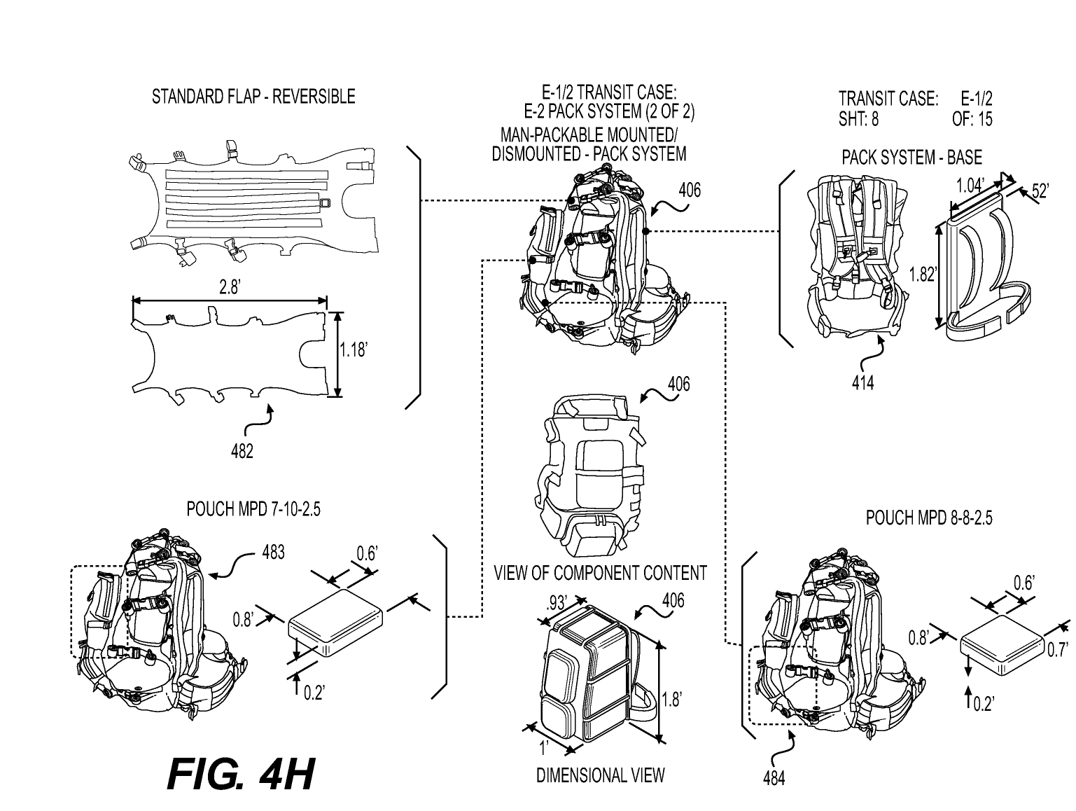

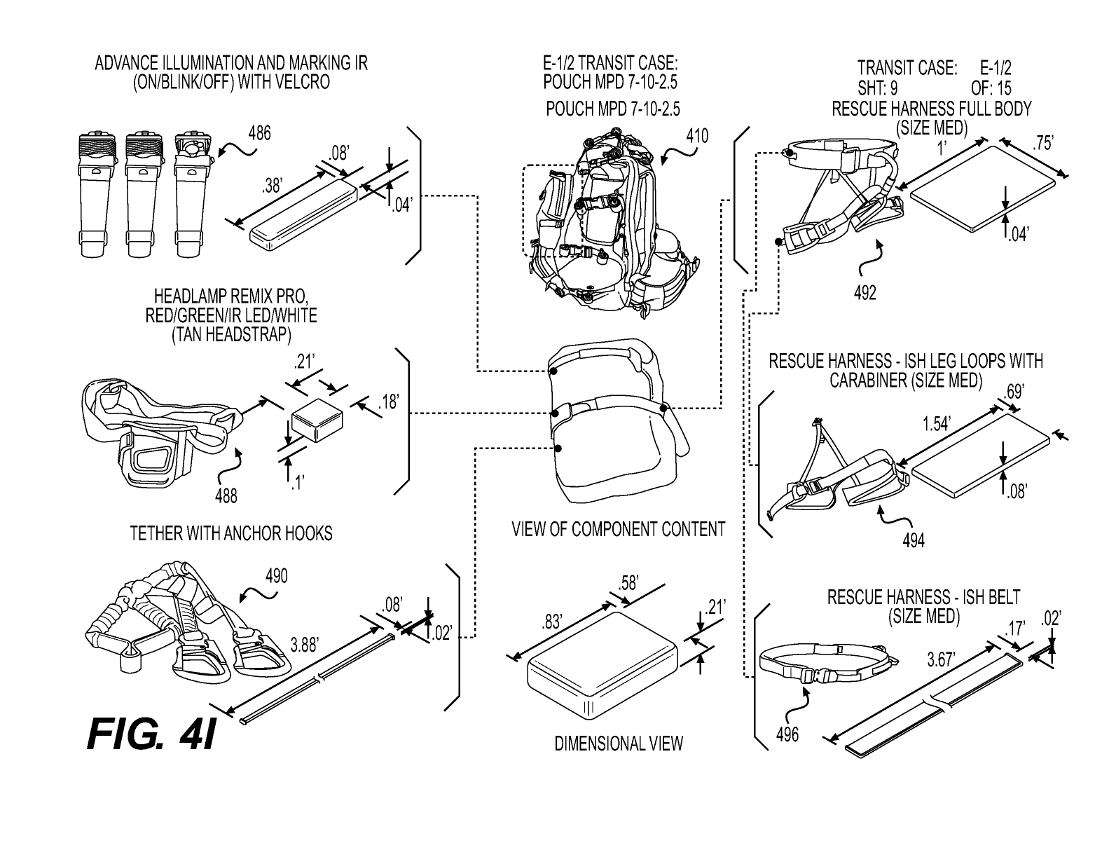

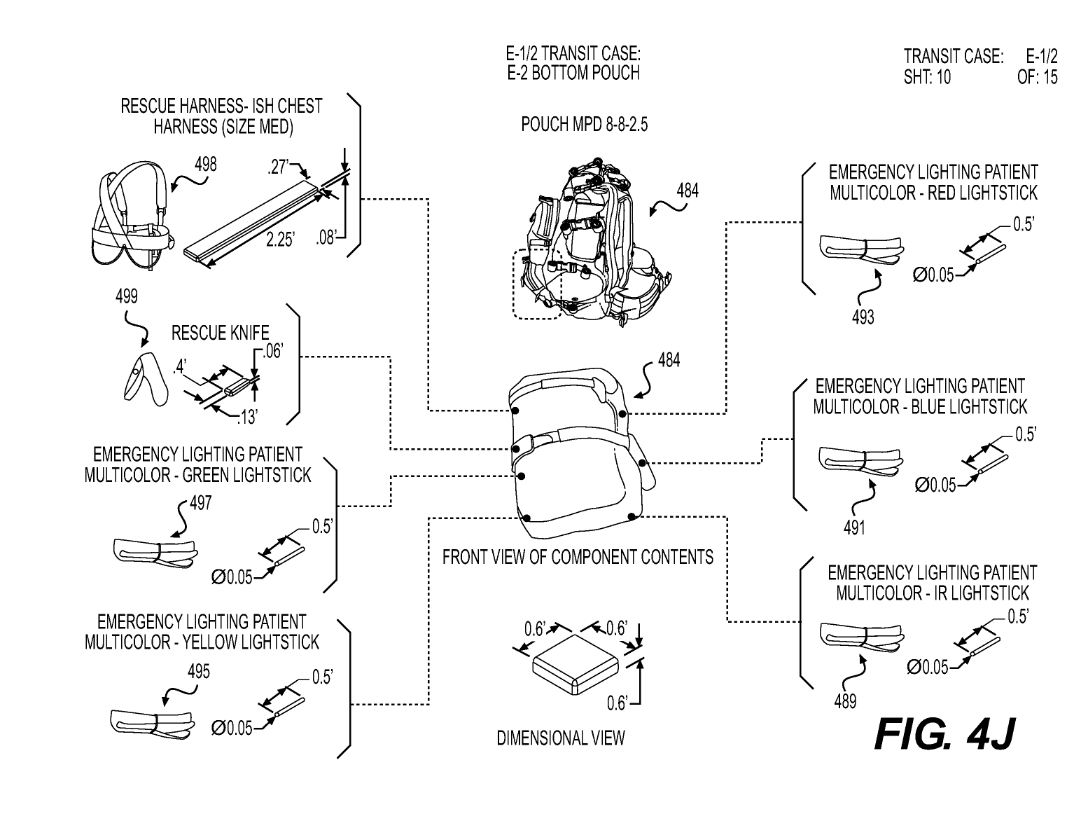

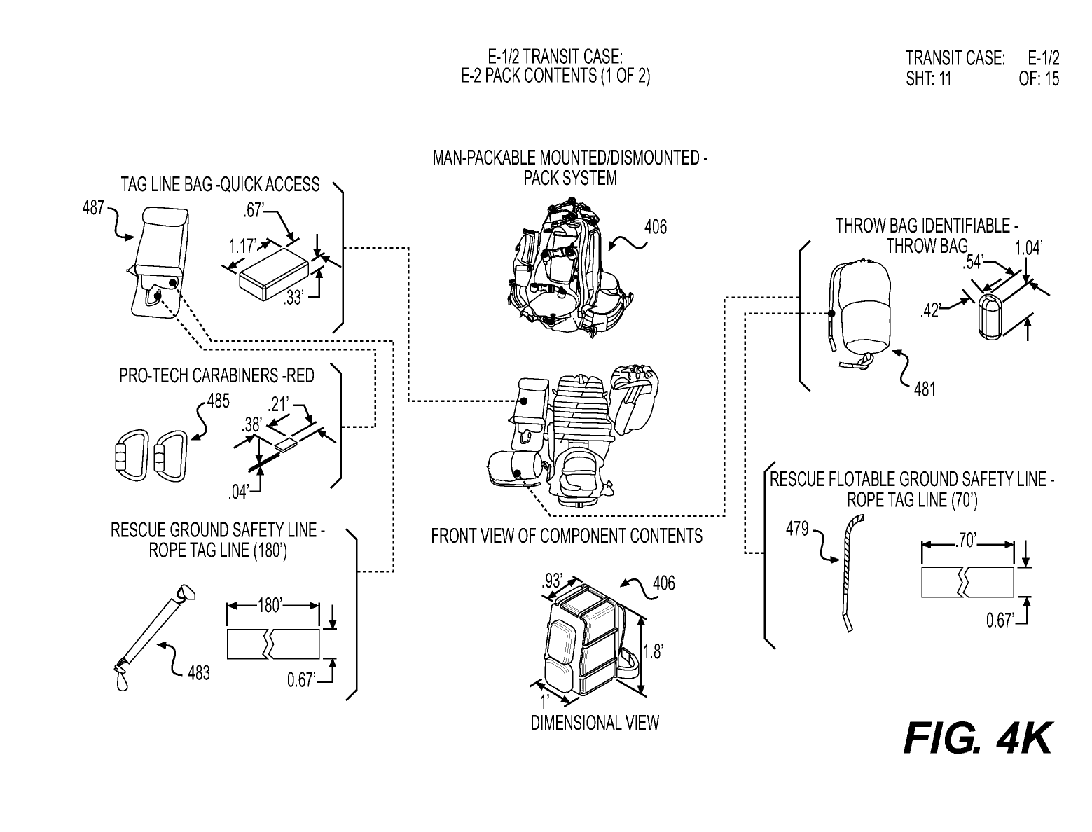

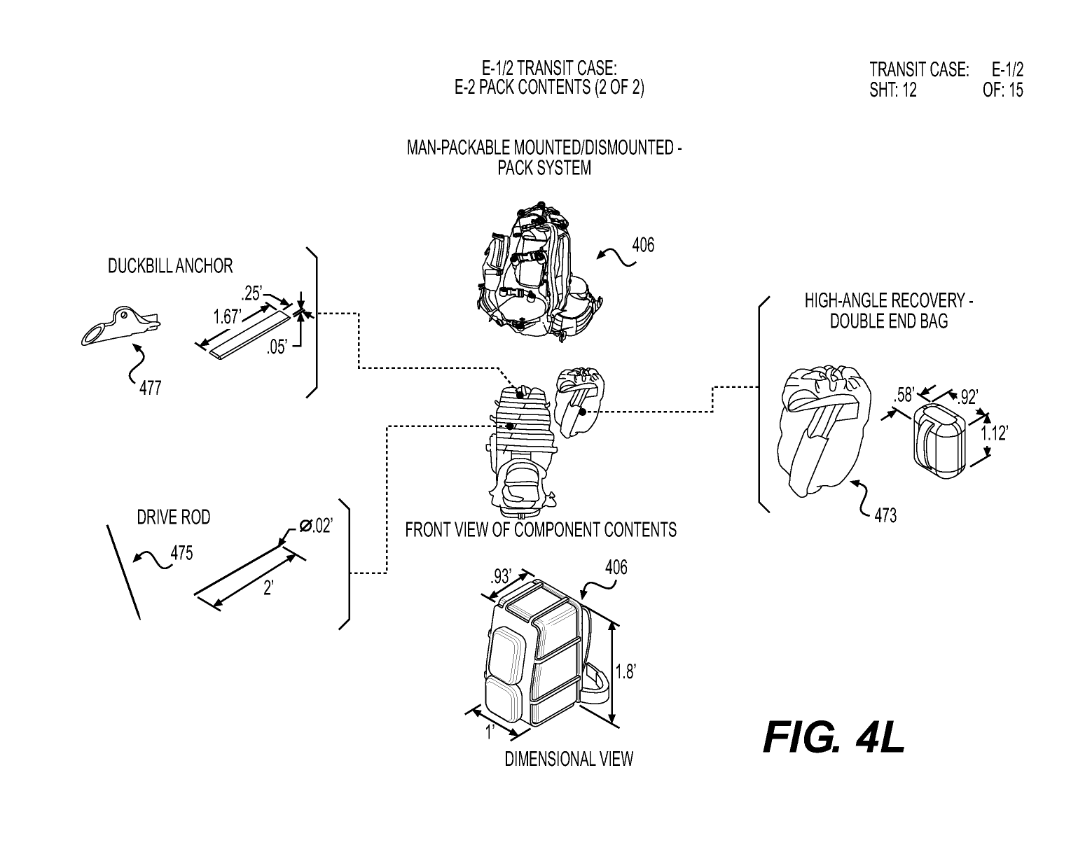

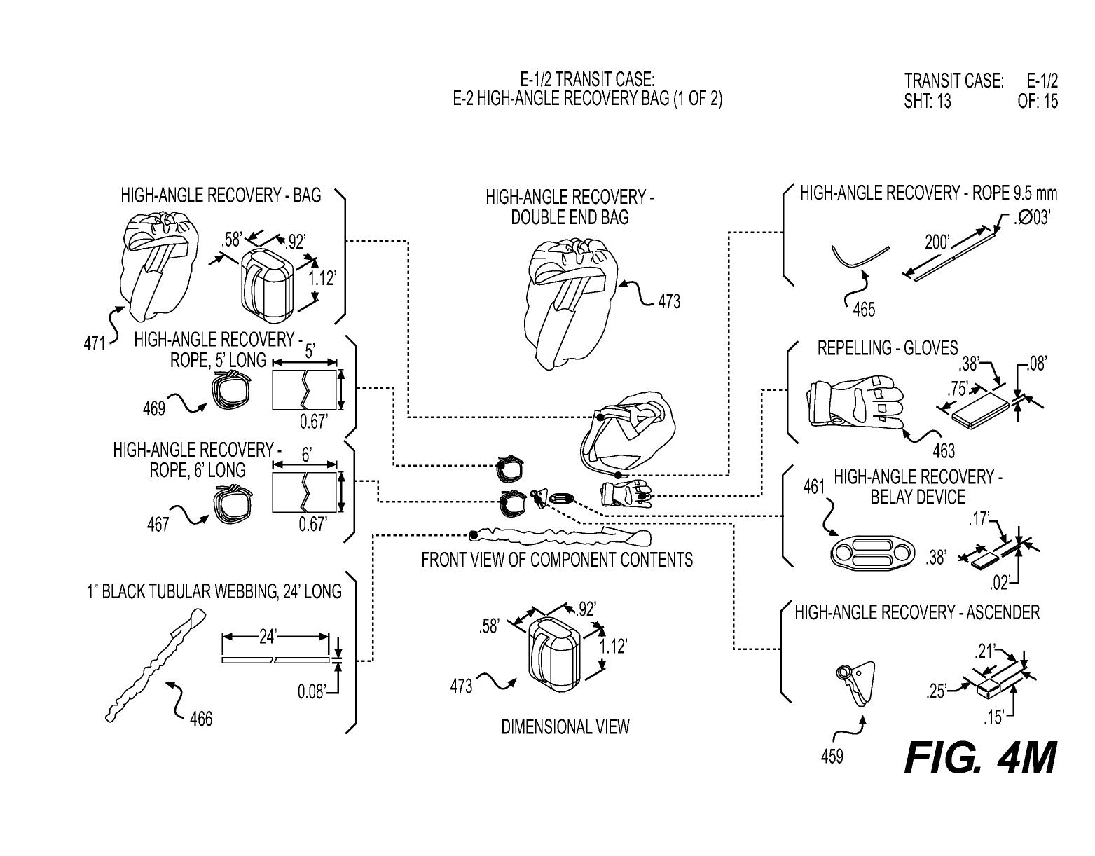

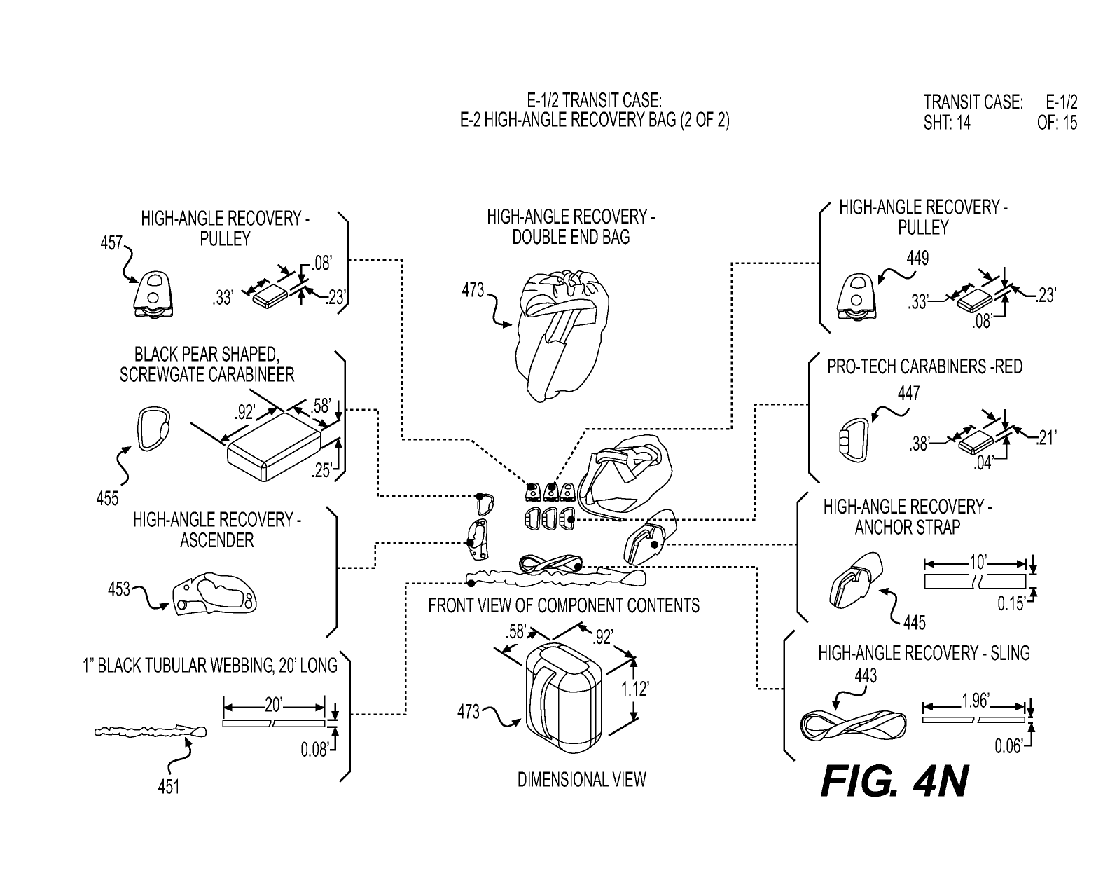

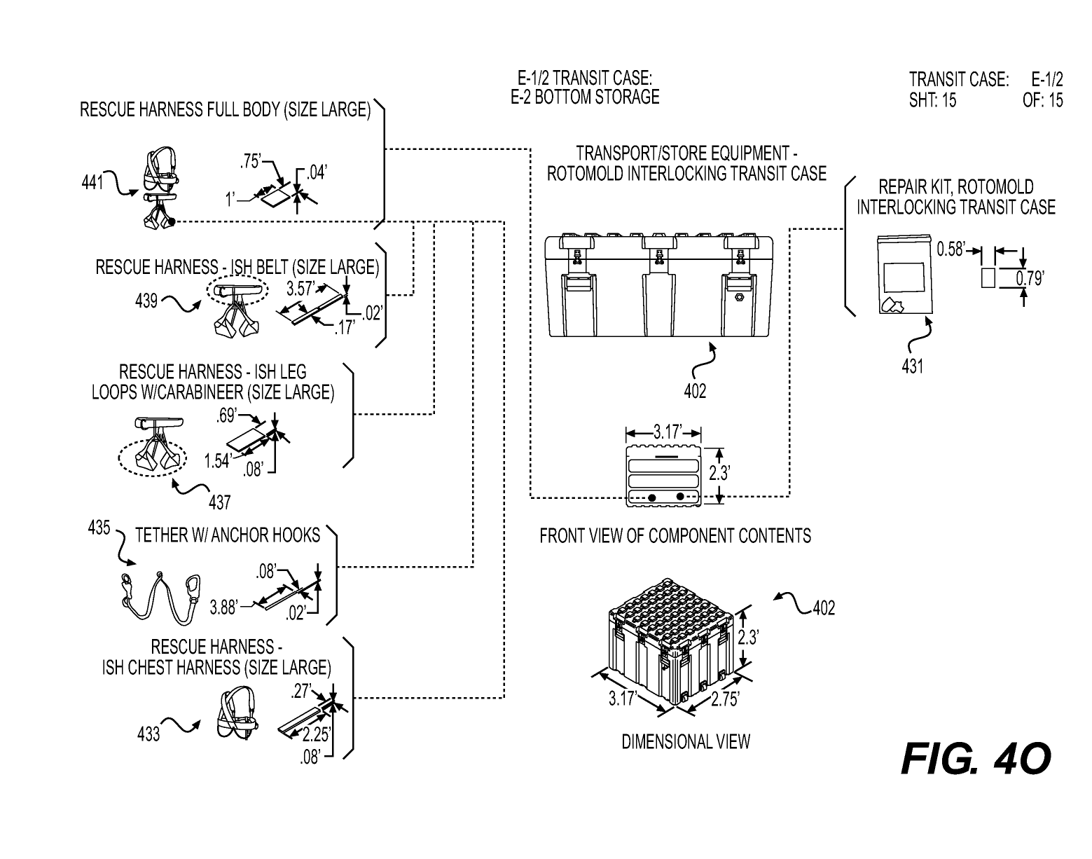

FIG. 4A through FIG. 10O describe the specific components of the complete set 100 in detail. National Stock Numbers (NSN's) are provided which identify what a specific component is. NSN's are known, public, standardized numbers that identify specific components, and the corresponding NSN's identify the specific components shown below. The items corresponding to NSN numbers may be looked up, for example, at http://www.dlis.dla.mil/webflis/pub/pub_search.aspx. There are also additional publicly available resources where NSN numbers may be found. These are specific examples using known NSN numbers, but other suitable components may be use. Multiple NSN numbers may indicate different versions and colors of the same type of item.

Dimensions are also provided on the figures. Some components use the same reference numeral to identify the component in different modules, but in this case, it may represent another instance of the same type of item in a different module or location. Although all components are described in the specification and the drawings, Appendix A below shows an additional table for ease of reference.

Generally, the contents are described in the following manner: 1) shipping container (i.e., transit case), 2) packs included in the shipping container/transit case, 3) outside pouches and individual parts of the pack system, 4) contents of each outside pocket, 5) opened pack system and suggested order of contents (in one implementation), and 6) contents of inside pouches and their sub-assemblies to individual items. The Figures and description of each kit (102-108) begins with the shipping container, i.e., transit case, that contains the kitted packs. The suggested order for the packs to be loaded into the container are displayed with a diagram of the pack, and a dashed line terminating with a dot shows where the pack should be placed.

The Figures and description of the pack systems (e.g., pack system 404 on FIG. 4B) typically begin with a top center label having the name of the pack. The pictures and descriptions start with the pack fully assembled and transition to the parts that make up the pack system. The outside pouches and where they are attached to the pack system may be highlighted by a dash square (e.g, FIG. 4C). Individual items in the contents of the outside pouch may be identified by a dashed line terminating with a dot.

The Figures transition from closed packs to pictures of the pack system open. Individual items and their locations for suggested assembly flow from top to bottom of the open pack system and are displayed with the same dashed line terminating with a dot. The same format for individual items located in the outside pouches of the pack system are used for individual items located inside the pack system. Individual items that are not assembled into the pack systems are identified after the Figures describing displaying the open pack systems. Their locations in the bottom of the transit cases are identified with a dashed line terminating with a dot. Other implementations and variations are possible besides the specifications shown in the Figures and description below.

FIG. 4A illustrates an exemplary E-1 and E-2 (E-1/2) transit case 108 of the extraction kit 102 (E) and its contents. Transport/storage equipment—rotomold interlocking transit case 402 (E-1) is shown containing man packable mounted/dismounted pack system 404 (E-1) and man packable mounted/dismounted pack system 406 (E-2). Transport/storage equipment—rotomold interlocking transit case 402 (E-1) may be NSN 814-01-606-1073, man packable mounted/dismounted pack system 404 (E-1) may be NSN 8465-01-606-3334 (or 3347, 3657 or 3666) and man packable mounted/dismounted pack system 406 may be NSN 8465-01-606-3696 (or 4250, 4254, 5493). Exemplary dimensions are shown on the Figure. As shown, the transport/storage equipment—rotomold interlocking transit case 402 (E-1) also contains E-1/2 transit case bottom storage, as well as documentation stored in the top portion. This documentation includes information regarding the contents of E-1/2 transit case 402 (E-1).

FIG. 4B illustrates an exemplary E-1 Pack system of the E-1/2 Transit Case 108. Man-Packable mounted/dismount-pack system 404 (E-1) is shown containing Pouch MPD 7-10-2.5 410 (E-1) and Pack System Base 414 (E-1). Pouch MPD 7-10-2.5 410 (E-1) may be NSN 8465-01-606-3373, 3409, 3436, or 3448. Pack system Base 414 (E-1) may be NSN 6515-01-603-8651, 8555, 3663, or 3671. As shown, the Man-packable mounted/dismount-pack system 404 (E-1) also contains non reversible extended flap 412 (the outside flap of the pack system) and lateral bag set (E-1) 416. Non reversible Extended flap-412 may be NSN 8465-01-606-3357, 3388, 3429 or 3445. Lateral Bag Set (E-1) 416 may be NSN 3465-01-606-3377, 3418, 3442, or 3764.

FIG. 4C depicts an exemplary E-1 Top Pouch of the E-1/2 Transit Case 108. Pouch MPD 7-10-2.5 410 (E-1) is shown containing Strap 418, Hoistable—Vertical Sling 420, Handles 422, Carabiner (Lite Allow Steel Screw Lock) 424, Hoistable—Horizontal Sling 426, Patient tie down strapstrap 428, Patient tie down strap 430, and E-1 OSS Pads Air Pocket/Zippsack small 432. E-1 OSS Pads Air Pocket/Zippsack Small 432 may be NSN 8465-01-606-3732, 3736, 3738, or 3744. The Strap 418 may be NSN 6530-01-262-4511. The Hoistable—vertical sling 420 may be NSN 6530-01-260-2699. The Handles 422 may be NSN 6530-01-260-7214. The Carabiner (Lite allow steel screw-lock) 424 may be NSN 6530-01-262-4510. The Hoistable-horizontal sling 426 may be NSN 6530-01-260-2700. The Patient tie down strapstrap 428 may be NSN 6530-01-536, 6294. The Patient tie down strap 430 may be NSN 6530-01-536-6278.

FIG. 4D shows an exemplary E-1 Lateral Bag Set of the E-1/2 Transit Case 108. This is the first of two figures showing this bag, the second being FIG. 4E below. Lateral Bag Set (E-1) 416 is shown containing Split upper extremity-SAM splint 36″ folded, grey/black 434, and SAM Junctional tourniquet 436. The Splint Upper extremity—same splint 36″ Folded, Grey/Black 434 may be NSN 6515-01-516-3104. The SAM junctional Tourniquet 436 may be NSN 6515-01-618-7475.

FIG. 4E illustrates an exemplary E-1 Lateral Bag Set of the E-1/2 Transit Case 108. This is the second of two figures showing this bag, the first being FIG. 4D. Lateral Bag Set (E-1) 416 is shown containing E-1 SM zippsack 438, extricate casualty water-kit 440, inflatable chest pad 442, bag 444, inflatable float log—left 446, inflatable float log-right 448, handles 450 and Cartridge 452. E-1 SM Zippsack 438 may be NSN 8465-01-606-3704, 3708, 3716, or 3717. The Extricate Casualty Water—Kit 440 may be NSN 6530-01-260-1227. The Inflatable—Chest pad 442 may be NSN 6530-01-260-1226. The bag 444 may be NSN 6530-01-262-1224. The Inflatable—Float log—L 446 may be NSN 6530-01-260-1223. The Inflatable—Float log—R 448 may be NSN 6530-01-260-1225. The Handles 450 may be NSN 6530-01-261-0448. The Cartridge 452 may be NSN 4220-01-607-1800, which inflates the float logs when pulled for water rescue.

FIG. 4F illustrates the pack contents of an exemplary E-1 Pack System. It is the first of two figures showing the contents of the pack system, the second being FIG. 4G below. Man-Packable mounted/dismount-pack system 404 is shown having contents of Package remains—bodybag 454, split lower extremity 456, OSS restraint pad (E-1) (Coyote Only) 458, Extrication Collar-AMBU perfit ace extrication collar-adult adjustable 460, Collapsible poleless hoistable litter-rescue kit—with cobra buckets 462, hoistable—litter—PJ sked 464 (which is a para-rescue jumper that is a hoistable patient litter, and 1″ black tubular webbing, 24′ long 466. The Package remains—bodybag 454 may be NSN 9930-01-607-194. The OSS Restraint pad (E1) (Coyote Only) 458 may be NSN 8465-01-606-3749. The 1″ Black tubular webbing, 24′ long 466 may be NSN 8465-01-607-6382.

FIG. 4G illustrates the pack contents of an exemplary E-1 Pack System. This is the second of two figures showing these pack contents, the first being FIG. 4F above. Man-Packable mounted/dismount-pack system 404 is shown having contents of Package Casualty Spinal injury-Oregon Spine Splint II 468, splint-restraint 470, splint-board 472, splint—collar strap 474, splint-pads 476, splint-body 478, and split-head pad 480. The Package casualty spinal injury—Oregon spine splint II 468 may be NSN 6530-01-265-3583. The Split-restraint 470 may be NSN 6530-01-265-3384. The Splint-board 472 may be NSN 6530-01-265-3384. The splint—collar strap 474 may be NSN 6530-01-265-3383. The Splint-Pads 476 may be NSN 6530-01-608-3968. The Splint-Head pad 480 may be NSN 6530-01-265-3385.

FIG. 4H shows an exemplary E-2 Pack System of the E-1/2 Transit Case 108. Man-Packable Mounted/dismounted—pack system 406 (E-2) is shown containing Standard flap-reversible 482 (which is an outside flap of the pack system), Pouch MPD 7-10-2.5 E-2 410, Pack System Base 414 (E-2), and Pouch MPD 8-8-2.5 484 (E-2). Standard flap-reversible 482 may be NSN 8465-01-606-3753, 4240, 4249 or 5514. Pouch MPD 8-8-2.5 484 (E-2) may be NSN 8465-01-606-3757, 4246, 4392, or 5515.

FIG. 4I illustrates an exemplary E-2 Top Pouch of the E-1/2 Transit Case 108. Pouch MPD 7-10-2.5 410 (E-2) is shown containing Advance illumination and marking IP (on/blink/off) with velcro 486, Headlamp remix pro, red/green/IR led, white (tan headstrap) 488, tether with anchor hooks 490, rescue harness full body (size med) 492, rescue harness—ISH (Integrated Stealth Harness) leg loops with carabiner (size med) 494 and rescue harness—ISH belt (size med) 496. The Advance illumination and marking IP (on/blink/off) w Velcro 486 may be NSN 6230-01-606-6635. The Headlamp remix pro, red/green/IR led, white (tan headstrap) 488 may be NSN 6230-01-606-1095. The Tether w/Anchor hooks 490 may be NSN 4240-01-606-2342. The Rescue Harness Full body (size mded) 492 may be NSN 4240-01-606-2308, The Rescue harness—ISH leg loops with carabiner (size med) 494 may be NSN 4240-01-606-2337. The Rescue harness—ISH belt (size med) 496 may be NSN 4240-01-606-2311.

FIG. 4J illustrates an exemplary E-2 Bottom pouch of the E-1/2 transit case 108. Pouch MPD 8-8-2.5 484 (E-2) is shown containing Rescue harness—ISH chest harness (size med) 498, Rescue Knife 499, Emergency lighting patient multicolor—green lightstick 497, emergency lighting patient multicolor—yellow lightstick 495, emergency lighting patient multicolor—red lightstick 493, Emergency lighting patient multicolor—blue lightstick 491, and Emergency lighting—IR lightstick 489. Rescue harness—ISH chest harness (size med) 498 may be NSN 4240-01-606-2345. Rescue Knife 499 may be NSN 4240-01-598-4341. Emergency lighting patient multicolor—green lightstick 497 may be NSN 6260-01-074-4229. Emergency lighting patient multicolor—yellow lightstick 495 may be NSN 6260-01-196-0136. Emergency lighting patient multicolor—red lightstick 493 may be NSN 6260-01-178-5559. Emergency lighting patient multicolor—blue lightstick 491 may be NSN 6260-01-178-5560. Emergency lighting—IR lightstick 489 may be NSN 6260-01-396-1704.

FIG. 4K illustrates the pack contents of an exemplary E-2 Pack system. This is the first of two figures showing these pack contents, the second being FIG. 4L below. Man-Packable mounted/dismount pack system 406 (E-2) is shown having contents of Tag line bag—quick access 487, Pro-tech carabiners—red 485, Rescue ground safety line—rope tag line (180′) 483, Throw bag identifiable—throw bag 481 which contains the water rescue rope, and Rescue floatable ground safety line—Rope Tag line (70′) 479. Tag Line Bag—quick access 487 may be NSN 8465-01-606-5539, 5541, 5544 or 5545. Pro-tech carabiners—Red 485 may be NSN 8465-01-606-5591. Rescue Ground safety line—rope tag line (180′) 483 may be NSN 4020-01-606-5578. Throw bag identifiable—throw bag 481 may be NSN 8465-01-606-5549, 5551, 5564, or 5565. Rescue floatable ground safety line—Rope Tag line (70′) 479 may be NSN 4020-01-606-5583.

FIG. 4L illustrates the pack contents of an exemplary E-2 Pack System. This is the second of two figures showing these pack contents, the first being FIG. 4K above. Man-Packable mounted/dismounted pack system 406 (E-2) is shown having contents of Duckbill anchor 477, Drive rod 475, and High-angle recovery—double end bag 473. Duckbill anchor 477 may be NSN 4010-01-606-1129. Drive rod 475 may be NSN 4030-01-606-1105, which provides a field expedient anchor for rescue hoist systems. High-angle recovery—double end bag 473 may be NSN 8465-01-606-5495, 5946, 5951 or 5955.

FIG. 4M illustrates an exemplary E-2 High-angle recovery bag. This is the first of two figures showing this bag, the second being FIG. 4N below. High-Angle recovery—Double end bag 473 (E-2) is shown containing High-Angle recovery bag 471, High-Angle recovery—Rope, 5′ long 469, High-Angle recovery—Rope, 6′ long 467, 1″ Black tubular webbing, 24′ long 466, High-Angle recovery—Rope 9.5 MM 465, Repelling-gloves 463, High-Angle recovery—Belay device 461, and High-Angle recovery—Ascender 459. High-Angle recovery—Double end bag 473 (E-2) may be NSN 8465-01-606-5495, 5946, 5951, or 5955. High-Angle recovery bag 471 may be NSN 8465-01-606-5498, 5868, 5870 or 5872. High-angle recovery—rope, 5′ long 469 may be NSN 4020-01-606-2407. High-Angle Recovery—Rope, 6′ long 467 may be NSN 4020-01-606-2485. Repelling gloves 463 may be NSN 8415-01-606-2512.

FIG. 4N illustrates an exemplary E-2 High-angle recovery bag. This is the second of two figures showing this bag, the first being FIG. 4M above. High-Angle recovery—Double end bag 473 (E-2) is shown containing High-angle recovery—pulley 457, Black Pear shaped, screwgate carabiner 455, High-Angle recovery—Ascender 453, 1″ black tubular webbing, 20′ long 451, High-angle recovery—pulley 449, Pro-tech carabiners—red 447, High-Angle recovery—Anchor strap 445, and High-Angle recovery—sling 443. Black Pear shaped, screwgate carabiner 455 may be NSN 8465-01-606-2494. High-angle recovery—Ascender 453 may be NSN 8465-01-579-3239. 1″ Black tubular webbing, 20′ long 451 may be NSN 8465-01-607-6381. High-Angle recovery—Anchor strap 445 may be NSN 8465-01-606-5889.

FIG. 4O illustrates an exemplary E-2 Bottom storage of the E-1/2 Transit case 108. Transport/Store Equipment—rotomold interlocking transit case 402 (E-1) is shown containing Rescue harness full body (size large) 441, Rescuse Harness—ISH belt (size large) 439, Rescue harness—ISH leg loops with carabiner (size large) 437, Tether with anchor hooks 435, Rescue harness—ISH chest harness (size large) 433, and repair kit in a rotomold interlocking transit case 431. Rescue harness full body (Size large) 441 may be NSN 4240-01-606-2515. Rescue harness—ISH belt (Size large) 439 may be NSN 4240-01-606-2517. Rescue Harness—ISH leg loops with carabiner (size large) 437 may be NSN 4240-01-606-2521. Tether with Anchor hooks 435 may be NSN 4240-01-606-2342. Rescue Harness—ISH chest Harness (size large) 433 may be NSN 4240-01-606-2532.

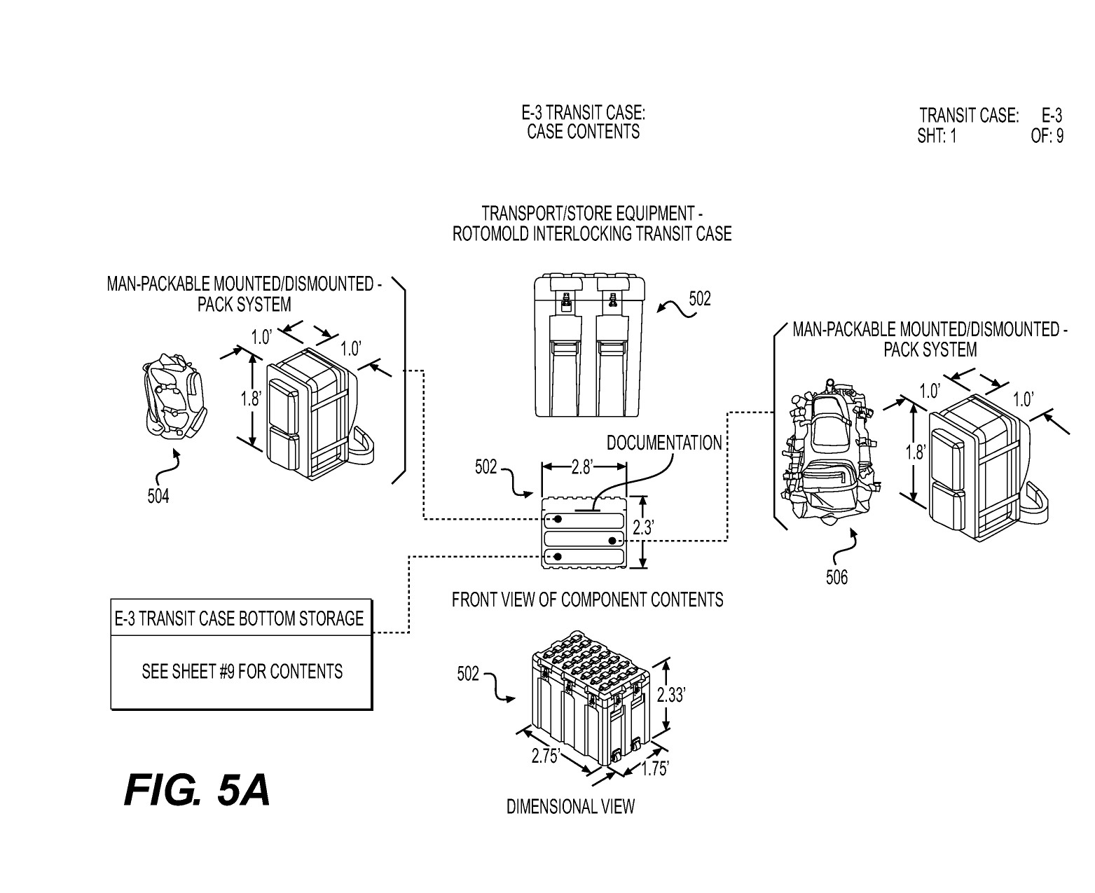

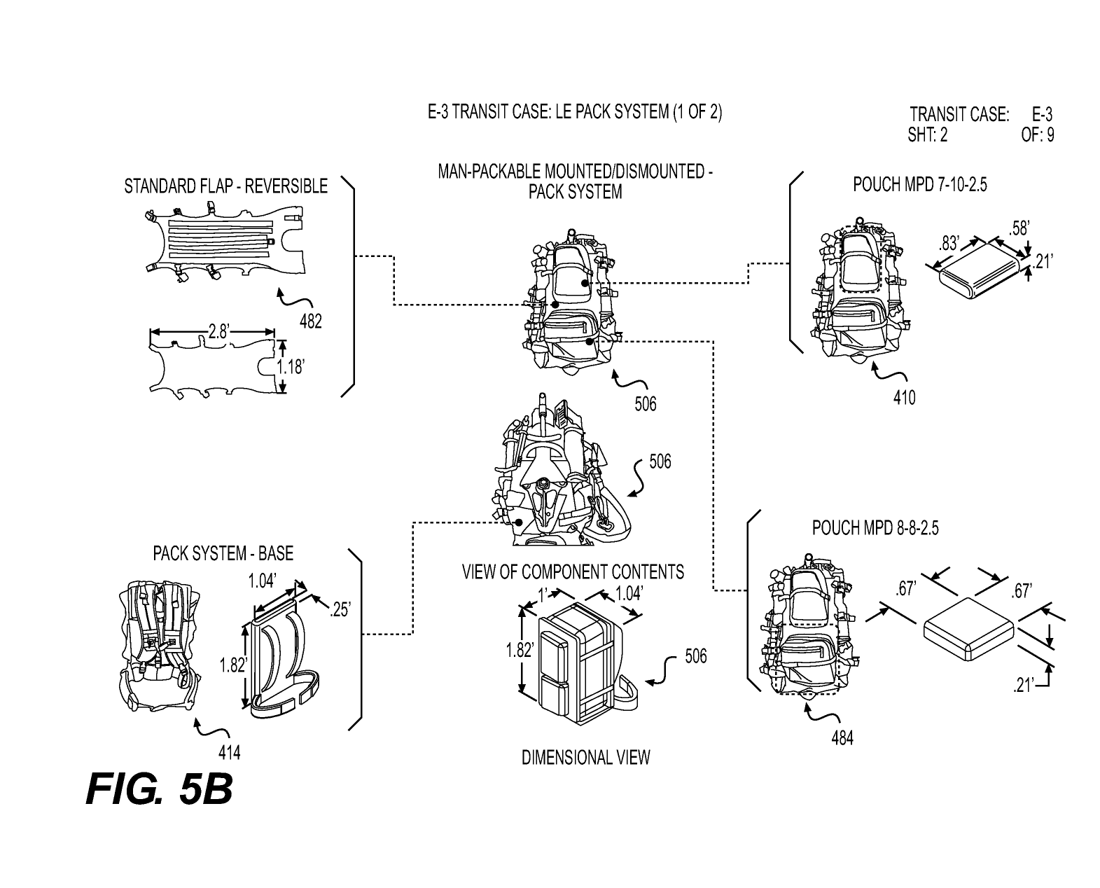

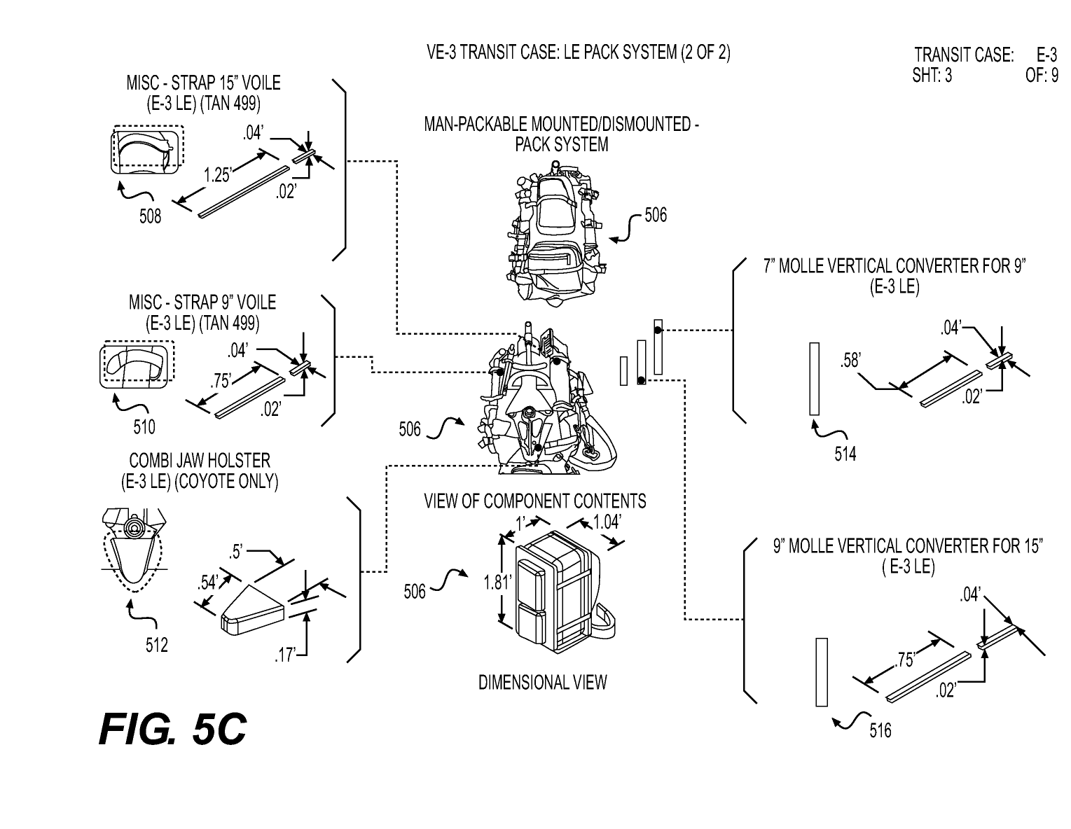

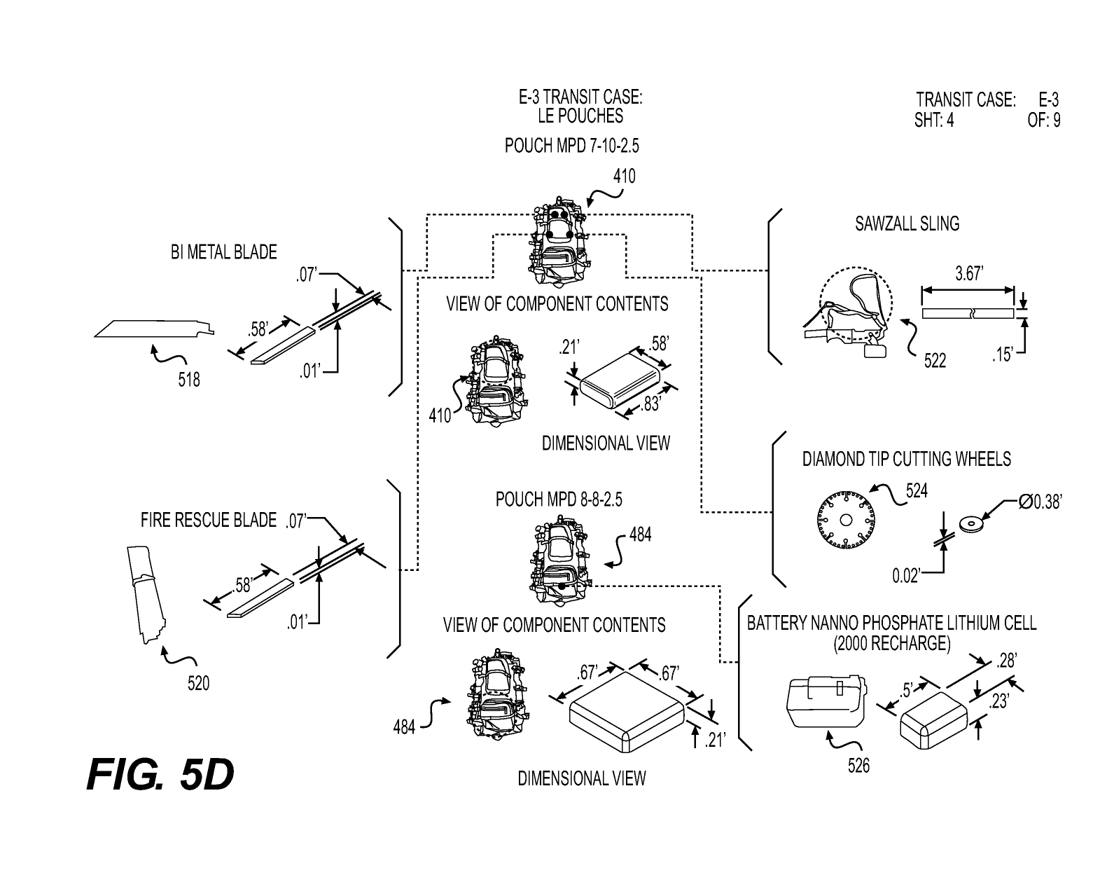

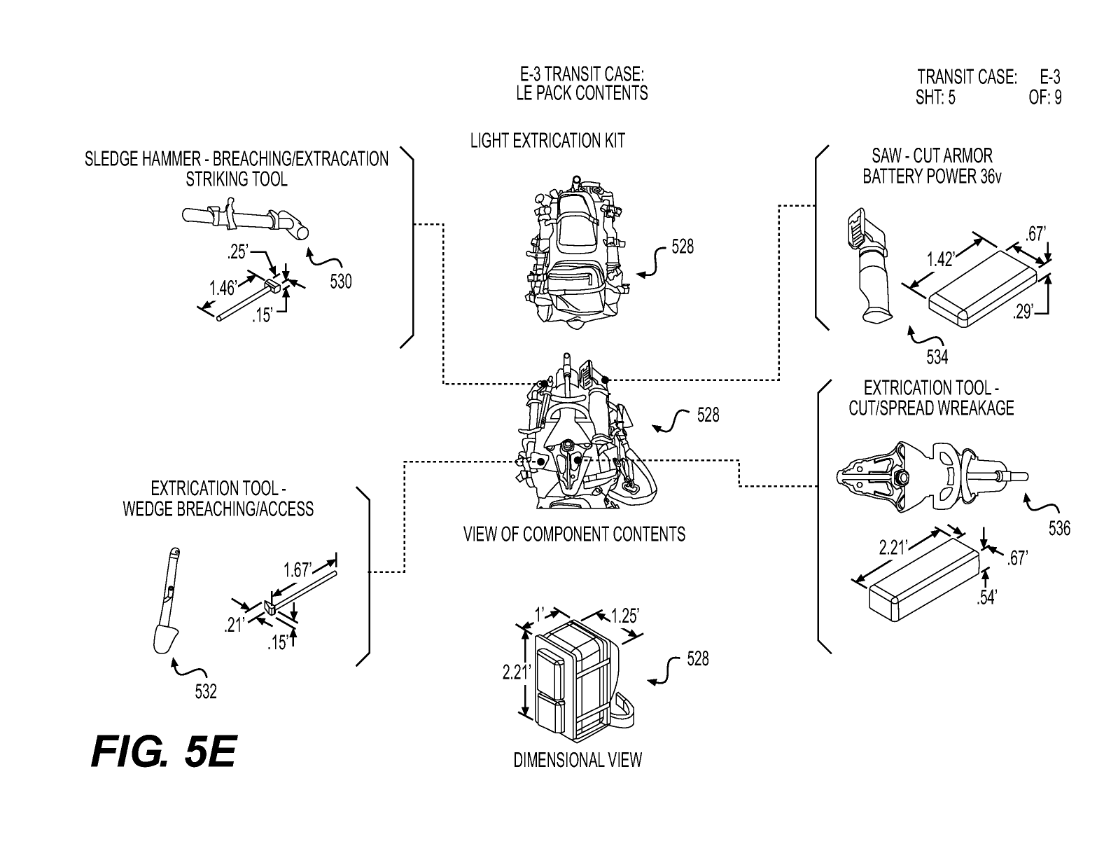

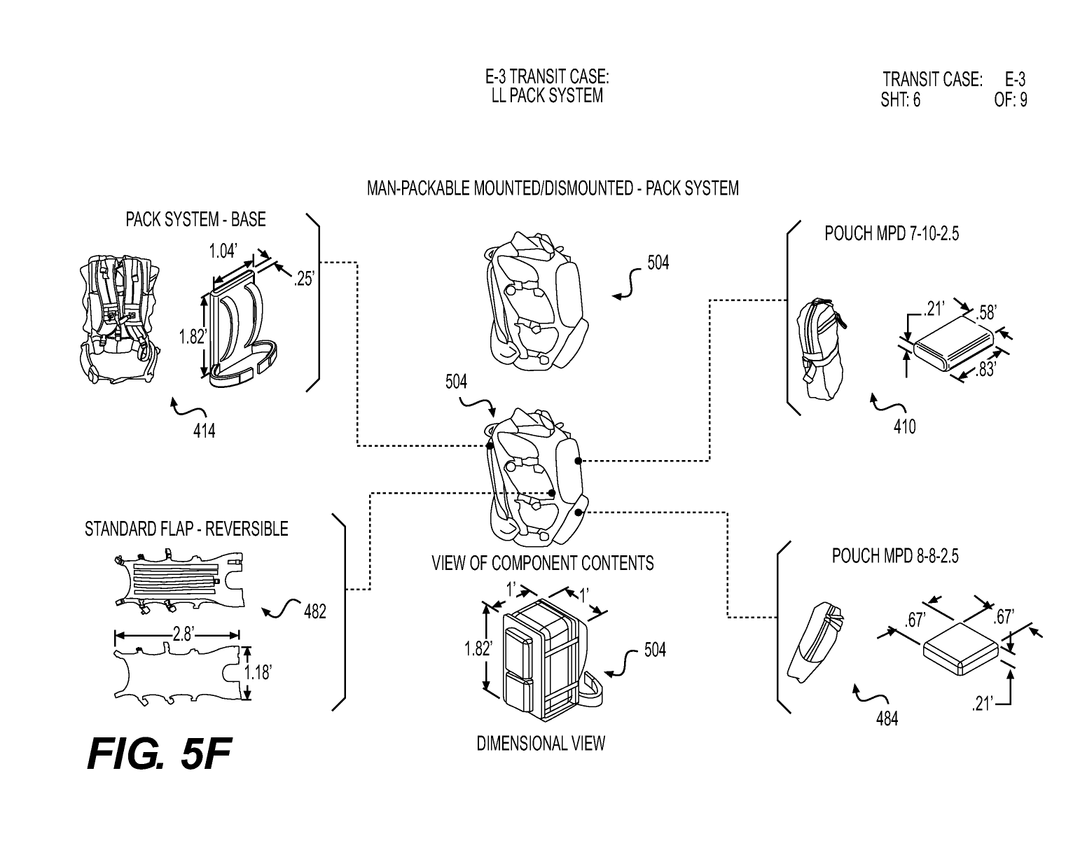

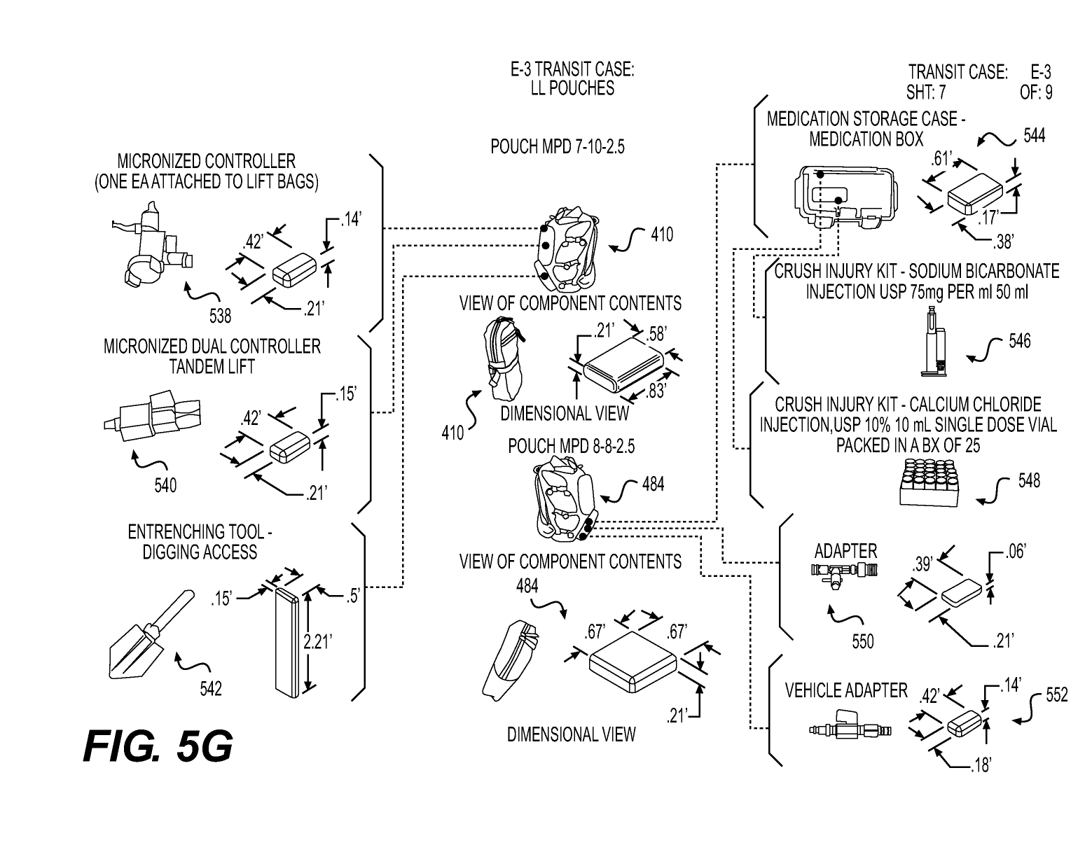

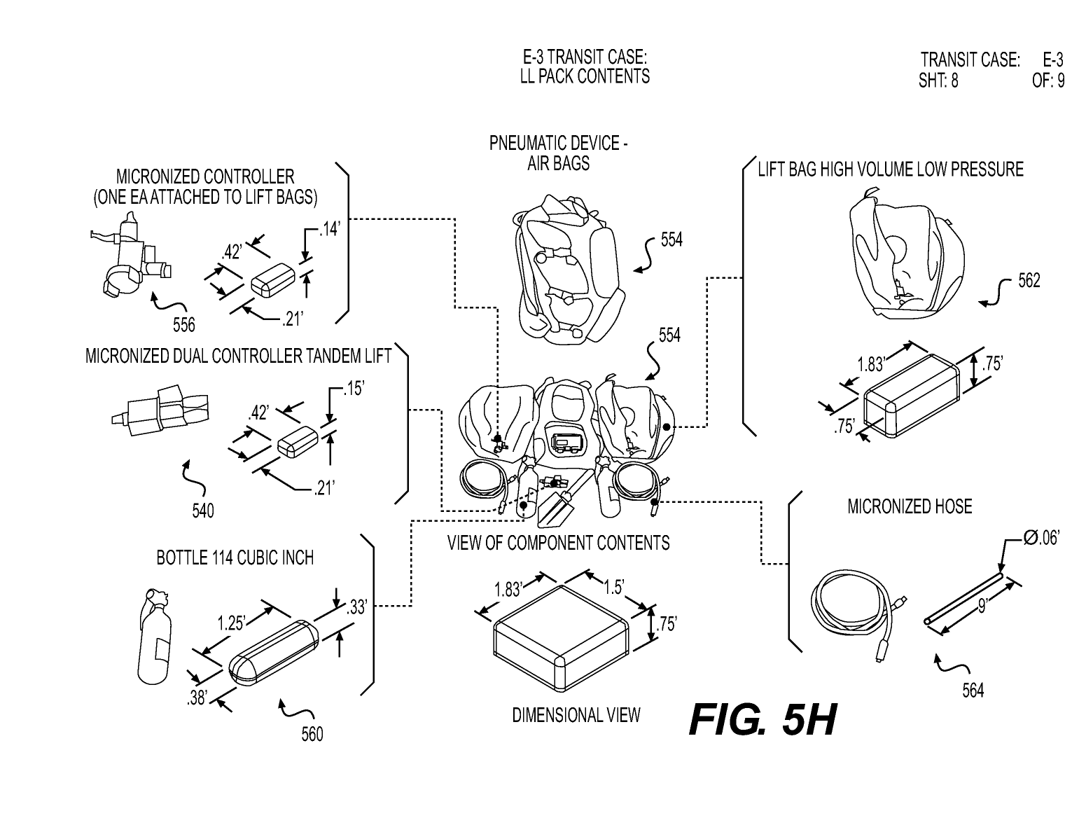

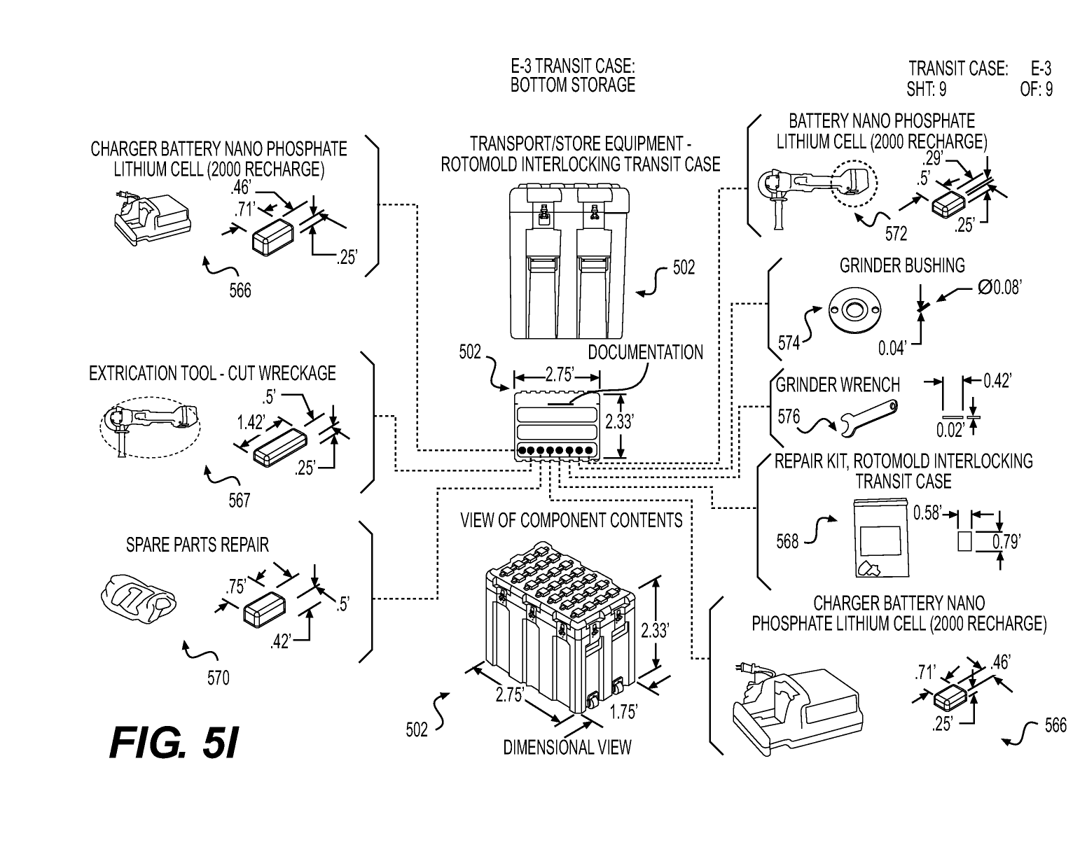

FIG. 5A illustrates the exemplary E-3 Transit case 114 and its contents. Transport/Store Equipment—Rotomold interlocking transit case 502 (E-3) is shown containing Man-Packable Mounted/Dismounted—Pack System 504 (E-3), and Man-Packable Mounted/Dismounted Pack System 506 (E-3). Transport/Store Equipment—Rotomold interlocking transit case 502 (E-3) may be NSN 8145-01-606-1154. Man-Packable Mounted/Dismounted—Pack System 504 (E-3) may be NSN 8465-01-606-5958, 5963, 5973, or 5974. Man-Packable Mounted/Dismounted—Pack System 506 (E-3) may be NSN 8465-01-606-5876, 5878, 5881 or 5937.

FIG. 5B illustrates the exemplary LE Pack System of the E-3 transit case 114. This is the first of two figures to show this pack system, the second being FIG. 5C below. Man-Packable Mounted/Dismounted—Pack System 506 (E-3) is shown containing Reversible Standard Flap 482, Pack System Base 414, Pound MPD 7-10-2.5 410 (E-3) and Pound MPD 8-8-2.5 484 (E-3).

FIG. 5C illustrates the exemplary LE (Light Extrication) Pack of the E-3 transit case 114. This is the second of two figures showing this pack system, the first being FIG. 5B above. Man-Packable Mounted/Dismounted—Pack System 506 (E-3) is shown containing Strap 15″ Voile (E-3 LE) (TAN 499) 508, Strap 9″ Voile (E-3 LE) (TAN 499) 510, Combi Jaw Holster (E-3 LE) (COYOTE) 512, 7″ Molle Vertical Converter for 9″ (E-3 LE) 514, and 9″ Molle Vertical Converter for 15″ (E-3 LE) 516. Strap 15″ Voile (E-3 LE) (Tan 499) 508 may be NSN 8465-01-606-5890. Strap 9″ Voile (E-3 LE) (Tan 499) 510 may be NSN 8465-01-606-5896. Combi Jaw Holster (E-3 LE) (COYOTE ONLY) 512 may be NSN 5140-01-606-5882. 7″ Molle Vertical Converter for 9″ (E-3 LE) 514 may be NSN 8465-01-606-5897, 5905, 5909 or 5921. 9″ Molle Vertical Converter for 15″ (E-3 LE) 516 may be NSN 8465-01-606-5893, 5903, 5906 or 5912.

FIG. 5D illustrates the exemplary LE Pouches of the E-3 transit case 114. Pouch MPD 7-10-2.5 410 is shown containing Bi Metal Blade 518, Fire Rescue Blade 520, Sawzall Sling 522, and Diamond Top cutting wheels 524. Pouch MPD 8-8-2.5 484 is shown containing Battery Nanno Phosphate Lithium Cell (2000 recharge) 526. Fire Rescue Blade 520 may be NSN 5130-01-606-7183. Sawzall Sling 522 may be NSN 8465-01-606-6576.

FIG. 5E illustrates the exemplary LE Pack Contents of the E-3 transit case 114. Light Extrication Kit 528 is shown containing Sledge Hammer—Breaching/Extrication Striking Tool 530, Extrication tool—Wedge Breaching/Access 532, Saw—Cut Armor Battery Power 36v 534, and Extrication Tool—Cut/Spread Wreakage 536. Light Extrication Kit 528 may be NSN 5180-01-606-7189. Sledge Hammer—Breaching/Extrication Striking Tool 530 may be NSN 5120-01-606-1798. Extrication tool—Wedge Breaching/Access 532 may be NSN 5120-01-606-7193. Saw—Cut Armor Battery Power 36v 534 may be NSN 5130-01-547-5641. Extrication Tool—Cut/Spread Wreakage 536 may be NSN 4240-01-605-2398.

FIG. 5F illustrates the exemplary LL (Light Lift) Pack System of the E-3 Transit Kit. Man-Packable Mounted/Dismounted—Pack System 504 is show containing Pack System Base 414, Reversible Standard Flap 482, Pouch MPD 7-10-2.5 410, and Pouch MPD 8-8-2.5 484.

FIG. 5G illustrates the exemplary LL Pouches of the E-3 transit case 114. Pouch MPD 7-10-2.5 410 is shown containing Micronized Controller (One EA Attached to lift bags) 538, Micronized Dual Controller Tandem Lift 540, and Entrenching Tool—Digging Access 542. Pouch MPD 8-8-2.5 484 is shown containing Medication Storage Case—Medication Box 544, Crush Injury Kit—Sodium Bicarbonate Injection USP 75 mg per ml 50 ml 546, Crush Injury Kit—Calcium Chloride Injection, USP 10%, 10 mL single dose vial packed in a box of 25 548, Adapter 550 and Vehicle Adapter 552. Micronized Controller (One EA Attached to lift bags) 538 may be NSN 4820-01-606-2546. Micronized dual controller tandem lift 540 may be NSN 4820-01-606-2576. Entrenching Tool—Digging Access 542 may be NSN 5120-01-606-1179. Crush Injury Kit—Sodium Bicarbonate Injection USP 75 mg per ml 50 ml 546 may be NSN NDC 517-063-925. Crush Injury Kit—Calcium Chloride Injection, USP 10%, 10 mL single dose vial packed in a box of 25 548 may be NSN NDC 517-271-025. Adapter 550 may be NSN 4240-01-606-6492. Vehicle adapter 552 may be NSN 4240-01-606-2584.

FIG. 5H illustrates the exemplary LL Pack Contents of the E-3 transit case 114. Pneumatic Device—Air Bags 554 is shown containing Micronized Controller (One EA attached to lift bags) 556, Micronized Dual Controller Tandem Lift 540, Bottle 114 cubic inch 560, Lift Bag High Volume Low Pressure 562, and Micronized Hose 564. Micronized Controller (One EA attached to lift bags) 556 may be NSN 4820-01-606-2546. Bottle 114 cubic inch 560 may be NSN 2590-01-606-2580. Lift Bag High Volume Low Pressure 562 may be NSN 5120-01-606-2563. Micronized Hose 564 may be NSN 4720-01-606-2572.

FIG. 5I illustrates the exemplary Bottom Storage of the E-3 transit case 114. Transport/Store Equipment—Rotomold Interlocking Transit Case 502 is shown containing Charger Battery Nano Phosphate Lithium Cell 566 (×2), Extrication Tool—Cut Wreckage 567, Spare parts repair 570, Battery Nano Phosphate Lithium cell (2000 recharge) 572, Grinder Bushing 574, Grinder Wrench 576, Repair Kit, and Rotomold Interlocking Transit Case 568. Extrication Tool—Cut Wreckage 567 may be NSN 5130-01-606-1195. Spare parts repair 570 may be NSN 5180-01-606-2586. Grinder Bushing 574 may be NSN 5310-01-606-1462. Grinder Wrench 566 may be NSN 5120-01-606-1457.

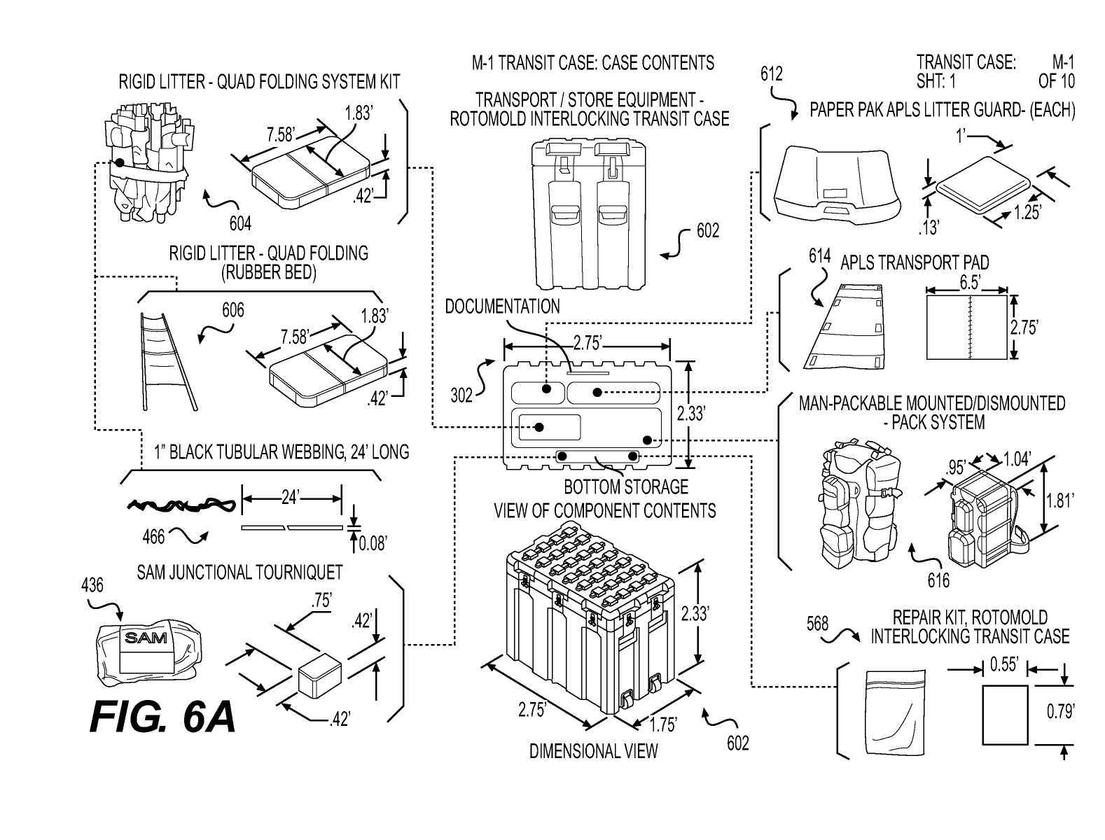

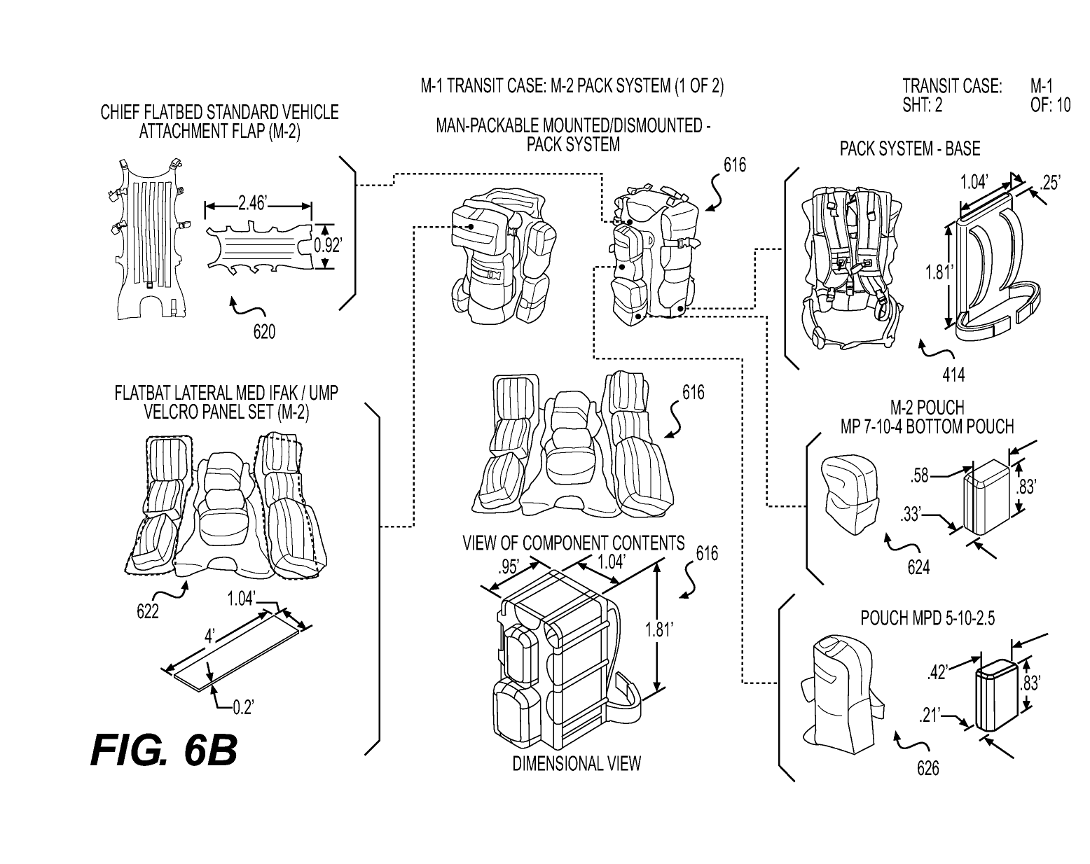

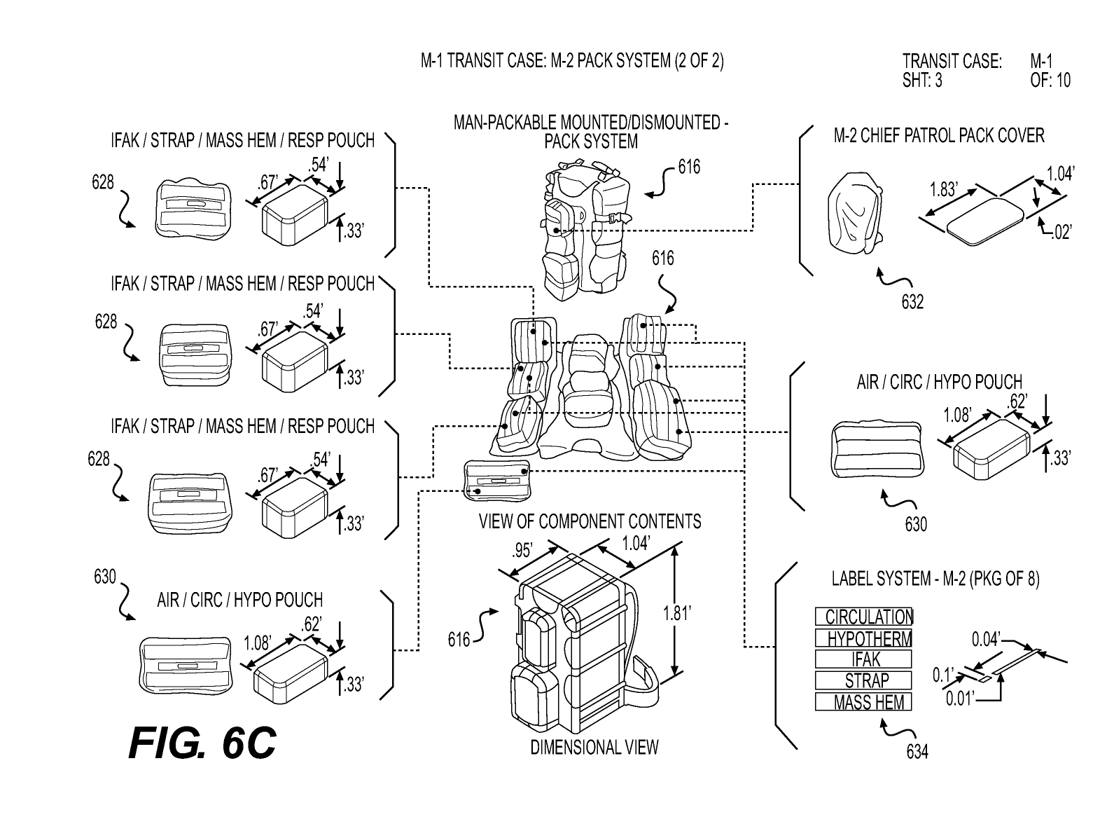

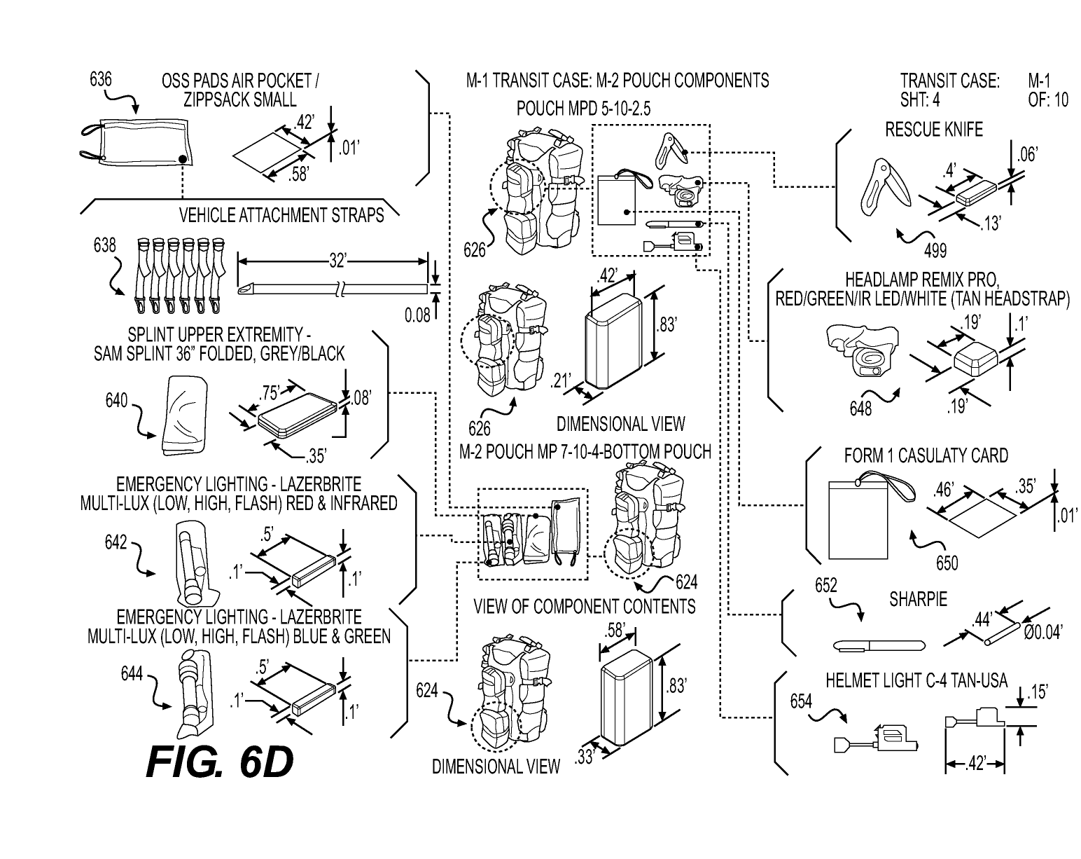

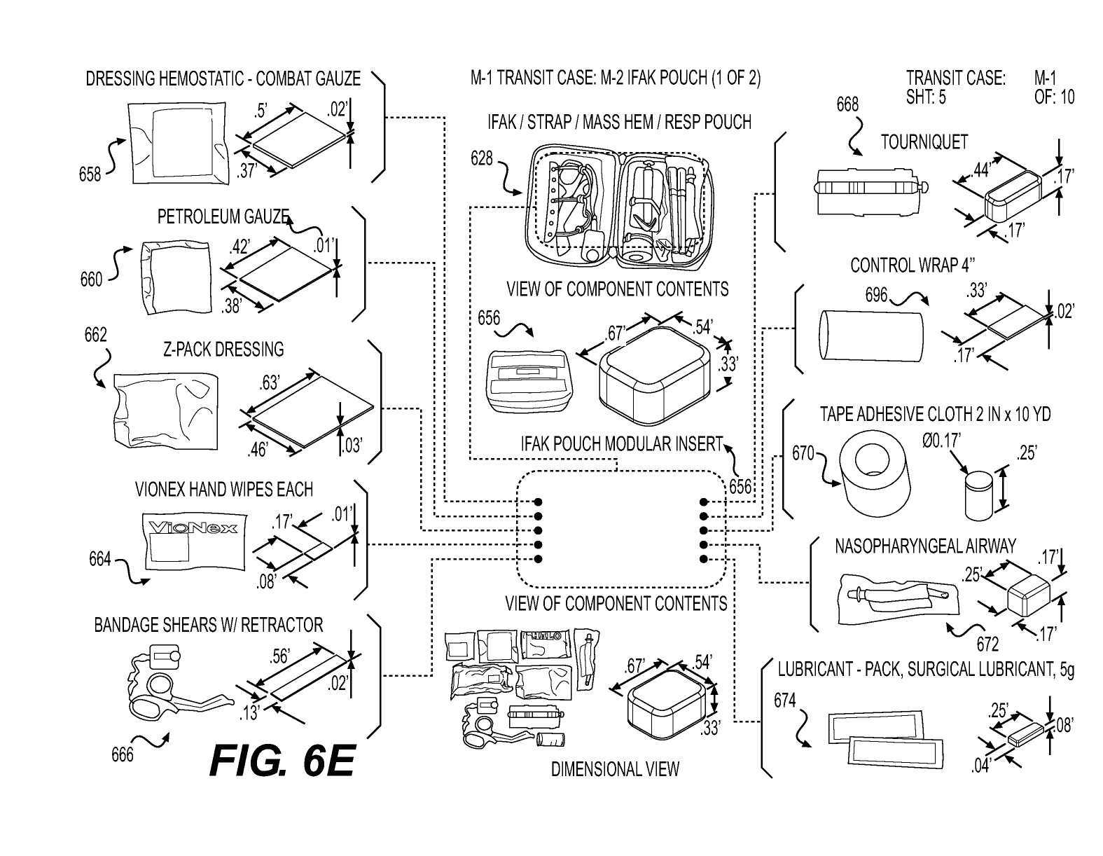

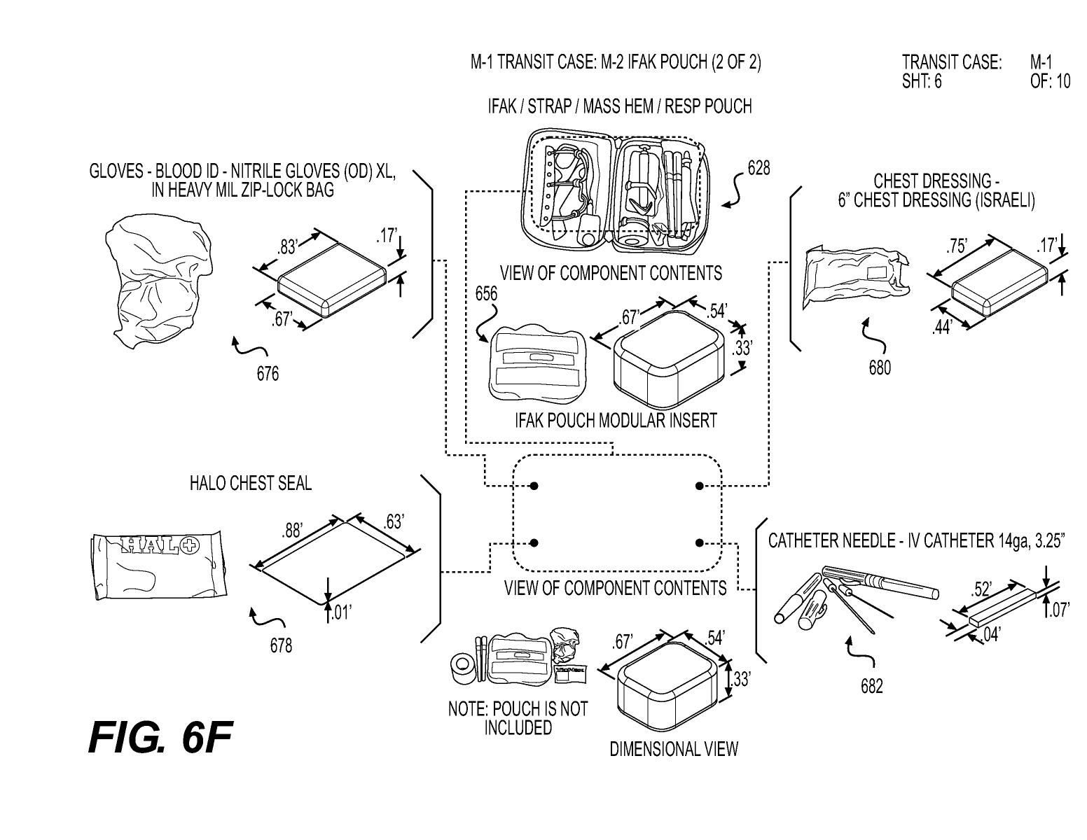

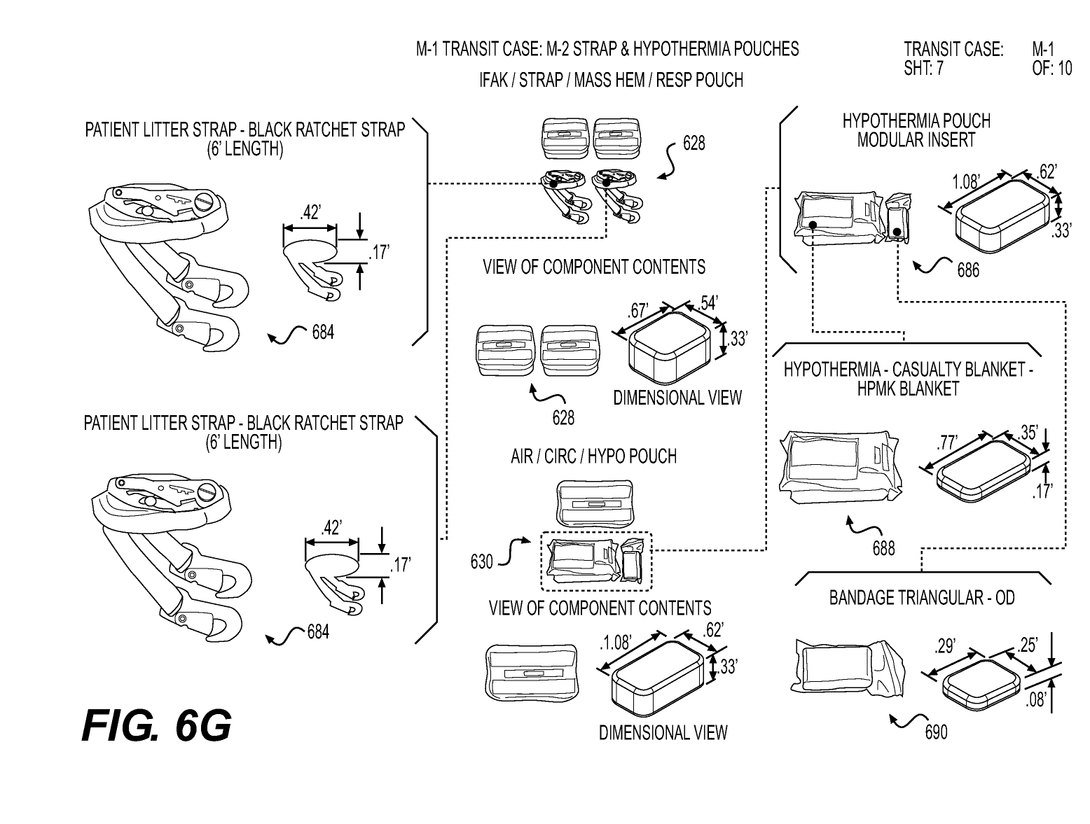

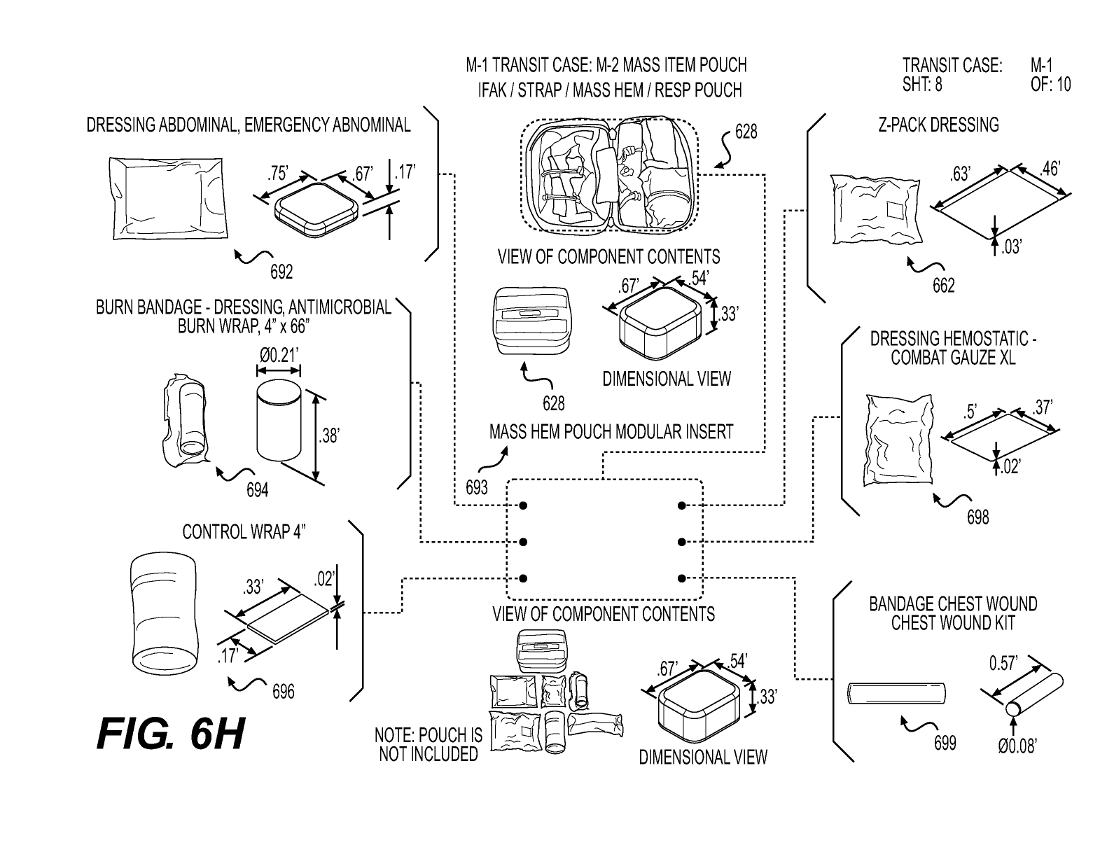

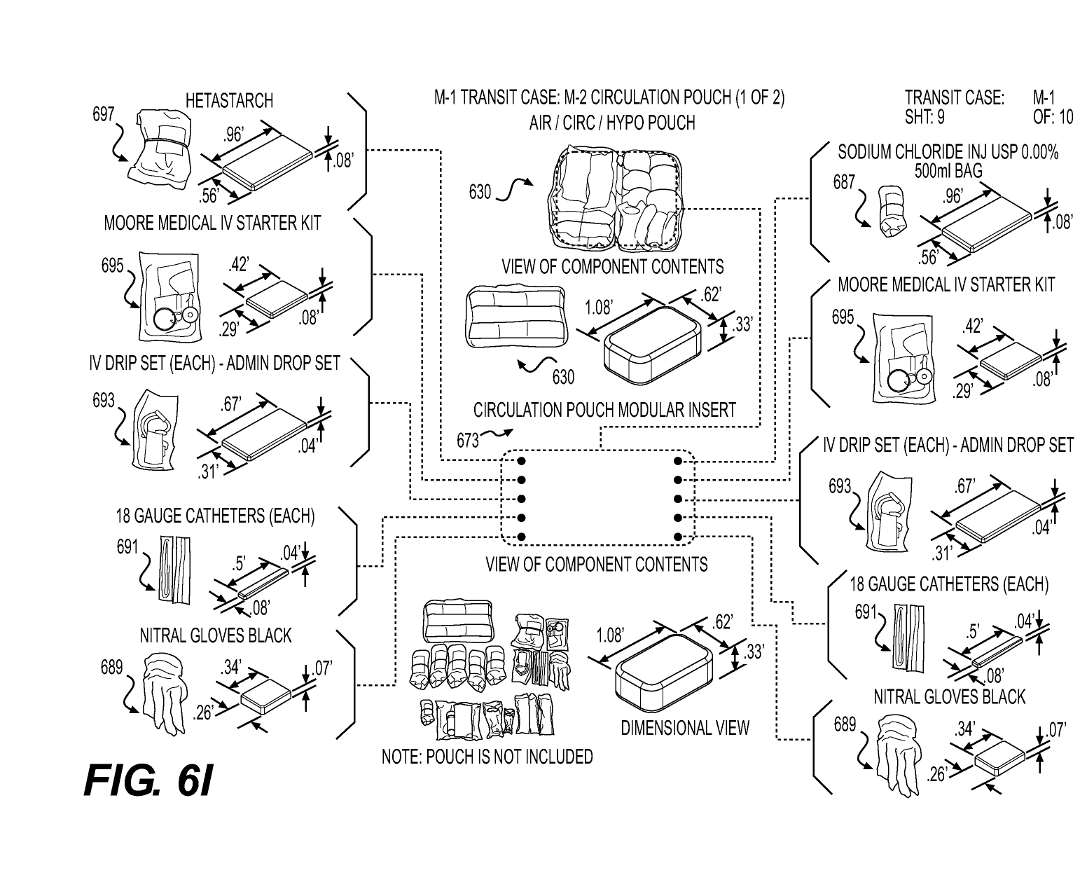

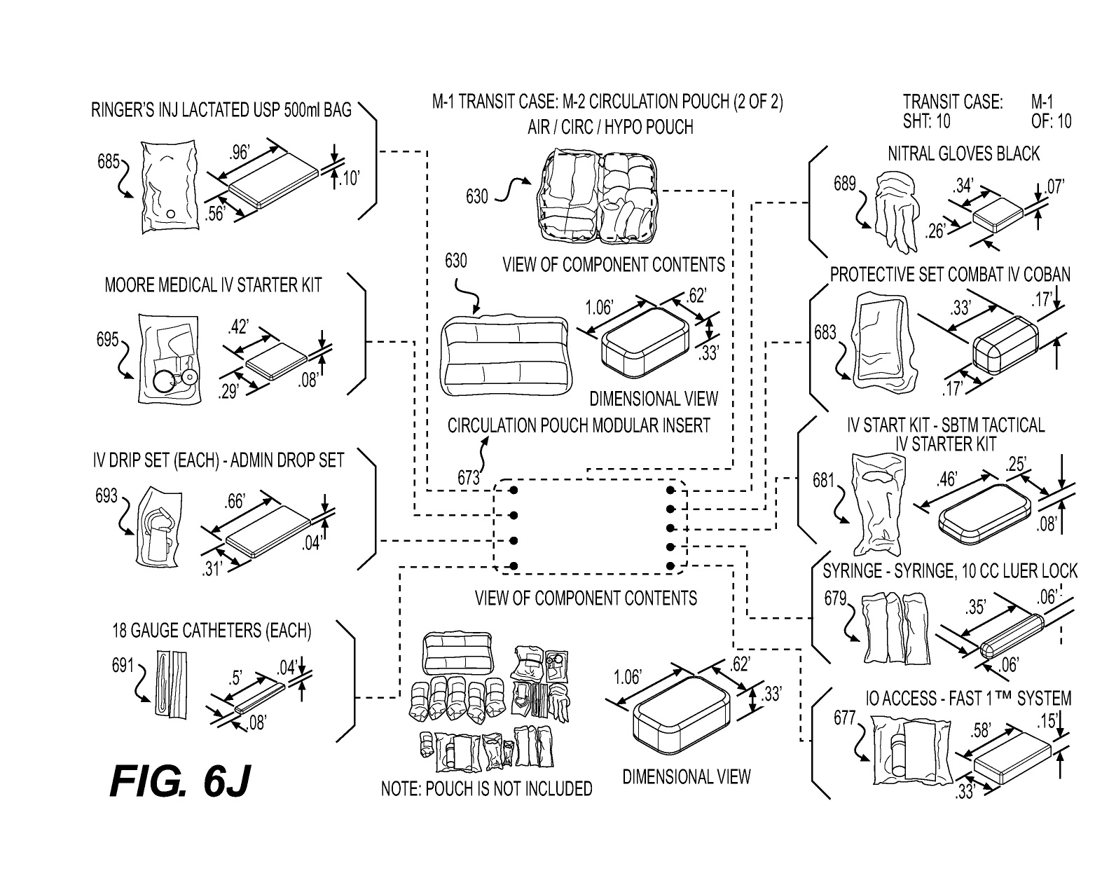

FIG. 6A illustrates the exemplary case contents of the M-1 transit case 120 of the mobility kit 104. Transport/Store Equipment—Rotomold Interlocking Transit Case 602 is shown containing Rigid Litter—Quad Folding System Kit 604 which is a rigid quad folding patient litter, Rigid Litter—Quad Folding (Rubber Bed) 606, 1″ black tubular webbing, 24′ long 466, Sam Junctional Tourniquet 436, Paper Pak APLS Litter Guard— (each) 612, APLS (Absorbent Patient Litter System) Transport Pad 614, Man-Packable Mounted/Dismounted—Pack System (M-2) 616, and Repair Kit in a Rotomold Interlocking Transit Case 568. Transport/Store Equipment—Rotomold Interlocking Transit Case 602 may be NSN 8145-01-606-1469. APLS transport pad 614 may be NSN 6530-01-557-1469. Man-Packable Mounted/Dismounted—Pack System (M-2) 616 may be NSN 8465-01-606-6475, 6482, 7294, or 7299.

FIG. 6B illustrates the exemplary M-2 Pack System of the M-1 transit case 120. This is the first of two figures showing this pack system, the second being FIG. 6C. Man-Packable Mounted/Dismounted—Pack System 616 is shown containing Chief Flatbed Standard Vehicle attachment flap (M-2) 620, Flatbed Lateral MED IFAK/UMP (Individual First Aid Kit) (Universal Medical Pouch) Velcro Panel Set (M-2) 622, Pack System—Base 414, M-2 Pouch MP 7-10-4 Bottom Pouch 624, and Pouch MPD 5-10-2.5 626. Chief Flatbed Standard Vehicle attachment flap (M-2) 620 may be NSN 8465-01-606-6509, 6542, 7235 or 7250. Flatbed Lateral MED IFAK/UMP Velcro Panel Set (M-2) 622 may be NSN 8465-01-606-7207, 7287, 7289 or 8645. M-2 Pouch MP 7-10-4 Bottom Pouch 624 may be NSN 8465-01-606-6562, 7225, 7240 or 8650. Pouch MPD 5-10-2.5 626 may be NSN 8465-01-606-6534, 7222, 7238 or 8649.

FIG. 6C illustrates the exemplary M-2 Pack System of the M-1 transit case 120. This is the second of two figures showing this pack system, the first being FIG. 6B. Man-Packable Mounted/Dismounted—Pack System 616 is shown containing IFAK/Strap/Mass Hem/Resp (Individual First Aid Kit, Strap, Massive Hemorrhage, or Respiratory) Pouch 628 (×3), Air/Circ/Hypo Pouch 630 (×2), M-2 Chief Patrol Pack Cover—632, and Label System—M-2 (PKG of 8) 634. IFAK/Strap/Mass Hem/Resp Pouch 628 may be NSN 6515-01-604-0418, 8465-01-606-7232, 7243 or 8653. Air/Circ/Hypo Pouch 630 may be NSN 6515-01-604-0410, 7233, 7245 or 8658. M-2 Chief Control Pack Cover—632 may be NSN 8465-01-606-1746, 1748, 1752 or 1755. Label System—M-2 (PKG of 8) 634 may be NSN 7690-01-606-9872.

FIG. 6D illustrates the exemplary M-2 Pouch Components of the M-1 transit case 120. Pouch MPD 5-10-2.5 626 is shown containing Rescue Knife 499, Headlamp Remix Pro, red/green/IR LED/white (Tan headstrap) 648, Form 1 Casualty Card 650, Sharpie 652, Helmet Light C-4 Tan-USA 654. M-2 Pouch MP 7-10-4 Bottom Pouch 624 is shown containing E-1 OSS Pads Air Pocket/Zippsack Small 636, Vehicle Attachment Straps—638, Splint Upper Extremity—Sam Splint 36″ Folded, Grey/Black 640, Emergency Lighting—LazerBrite Multi-Lux (low, high, flash)—Red & Infrared 642, and Emergency Lighting—LazerBrite Multi-Lux (low, high, flash)—Blue & Green 644. E-1 OSS Pads Air Pocket/Zippsack Small 636 may be NSN 8465-01-606-3732, 3736, 3738 or 3744. Vehicle attachment straps-638 may be NSN 3990-01-606-7211, 7293, 7304 or 9870. Splint Upper Extremity—Sam Splint 36″ Floded, Grey/Black 640 may be NSN 6551-01-516-3104. Emergency Lighting—LazerBrite Multi-Lux (low, high, flash)—Red & Infrared 642 may be NSN 6230-01-606-1510. Emergency Lighting—LazerBrite Multi-Lux (low, high, flash)—Blue & Green 644 may be NSN 6230-01-606-1540. Headlamp Remix Pro, Red/Green/IR LED/White (Tan headstrap) 648 may be NSN 6230-01-606-1095. Form 1 Casualty Card 650 may be NSN 6515-01-587-1700. Helmet Light C-4 Tan-USA 654 may be NSN 6230-01-606-1473.

FIG. 6E illustrates the exemplary M-2 IFAK Pouch of the M-1 transit case 120. This is the first of two figures showing this pouch, the second being FIG. 6F below. IFAK/Strap/Mass Hem/Resp Pouch 628 is shown containing IFAK Pouch Modular Insert 656. IFAK Pouch Modular Insert 656 is shown as containing Dressing Hemostatic—Combat Gauze 658, Petroleum Glaze 660, Z-Pack Dressing 662, Vionex Hand Wipes Each 664, Bandage Shears with Retractor 666, Tourniquet 668, Tape Adhesive Cloth 2 in×10 yd 670, Nasopharyngeal Airway 672, and Surgical Lubricant Pack, 5g 674. IFAK Pouch Modular Insert 656 may be NSN 6545-01-607-7796. Dressing Hemostatic—Combat Gauze 658 may be NSN 6510-01-562-3325. Petroleum Gauze 660 may be NSN 651-01-608-2536. Z-Pack Dressing 662 may be NSN 6510-01-527-8329. Tourniquet 668 may be NSN 6515-01-530-7015.

FIG. 6F illustrates the exemplary M-2 IFAK Pouch of the M-1 transit case 120. This is the first of two figures showing this pouch, the first being FIG. 6F above. IFAK/Strap/Mass Hem/Resp Pouch 628 is shown as containing IFAK Pouch Modular Insert 656. IFAK Pouch Modular Insert 656 is shown containing Gloves—Blood ID—Nitrile Gloves (OD), XL, In heavy military zip-lock bag 676, Halo Chest Seal 678, Chest Dressing—6″ Chest Dressing (Israeli) 680, Catheter Needle, IV Catheter 14GA, 3.25″ 682. Gloves—Blood ID—Nitrile Gloves (OD), XL, In heavy mil zip-lock bag 676 may be NSN 6515-01-521-7505. Halo Chest Seal 678 may be NSN 6510-01-581-0553. Chest Dressing—6″ Chest Dressing (Israeli) 680 may be NSN 6510-01-492-2275. Catheter Needle, IV Catheter 14GA, 3.25″ 682 may be NSN 6515-01-541-0635.

FIG. 6G illustrates the exemplary M-2 Strap & Hypothermia Pouches of the M-1 transit case 120. IFAK/Strap/Mass Hem/Resp Pouch 628 is shown containing Patient Litter Strap—Black ratchet strap (6′ length) 684 (×2). Air/Circ/Hypo Pouch 630 is shown containing Hypothermia Pouch Modular Insert 686. Hypothermia Pouch Modular Insert 686 is shown containing Hypothermia—Casualty Blanket—HPMK Blanket 688 and Bandage Triangular—OD 690. Patient Litter Strap—Black ratchet strap (6′ length) 684 may be NSN 8465-01-606-9880. Hypothermia Pouch Modular Insert 686 may be NSN 6545-01-607-7817. Hypothermia—Casualty Blanket—HPMK (Hypothermia Prevention and Management Kit) Blanket 688 may be NSN 6515-01-532-8056. Bandage Triangular—OD (Olive drab, color) 690 may be NSN 6510-00-201-1755.

FIG. 6H illustrates the exemplary M-2 Mass Hem Pouch of the M-1 transit case 120. IFAK/Strap/Mass Hem/Resp Pouch 628 is shown containing Mass Hem Pouch Modular Insert 693. Mass Hem Pouch Modular Insert 693 is shown containing Dressing Abdominal, Emergency Abdominal 692, Burn Bandage—Dressing, Antimicrobial Burn Wrap, 4″×66″ 694, Control Wrap 4″ 696, Z-Pack Dressing 662, Dressing Hemostatic—Combat Gauze XL 698, and Bandage Chest Wound Chest Wound Kit 699. Dressing Abdominal, Emergency Abdominal 692 may be NSN 6510-01-586-4314. Mass Hem Pouch Modular Insert 693 may be NSN 6545-01-607-7809. Burn Bandage—Dressing, Antimicrobial Burn Wrap, 4″×66″ 694 may be NSN 6510-01-522-2173. Control Wrap 4″ 696 may be NSN 6510-01-609-1057. Dressing Hemostatic—Combat Gauze XL 698 may be NSN 6510-01-600-4705. Bandage Chest Wound Chest Wound Kit 699 may be NSN 6515-01-600-4971.

FIG. 6I illustrates the exemplary M-2 Circulation Pouch of the M-1 transit case 120. This is the first of two figures showing this pouch, the second being FIG. 3J Air/Circ/Hypo Pouch 630 is shown containing Circulation Pouch Modular Insert 673. Circulation Pouch Modular Insert 673 is shown containing Hetastarch 697, Moore Medical IV Starter Kit 695 (×2), IV Drip Set (each)—Admin Drop Set 693 (×2), 18 Gauge Catheters (each) 691 (×2), Nitrile Gloves Black 689 (×2) and Sodium Chloride Inj USP 0.900% 500 ml Bag 687. Circulation Pouch Modular Insert 673 may be NSN 6545-01-607-7845. Hetastarch 697 may be NSN NDC 0409-1555-54. Sodium Chloride Inj USP 0.900% 500 ml Bag 687 may be NSN NDC 0409-7983-03.

FIG. 6J illustrates the exemplary M-2 Circulation Pouch of the M-1 transit case 120. This is the second of two figures showing this pouch, the first being FIG. 6I. Air/Circ/Hypo Pouch 630 is shown containing Circulation Pouch Modular Insert 673. Circulation Pouch Modular Insert 673 is shown containing Ringer's Inj Lactated USP 500 ml Bag 685, Moore Medical IV Starter Kit 695, IV Drip Set (each) Admin Drop set 693, 18 Gauge Catheters (each) 691, Nitrile Gloves Black 689, Protective Set Combat IV Coban 683, IV Start Kit—SBTM Tactical IV Starter Kit 681, Syringe—Syringe, 10 CC Luer Lock 679, IO Access—FAST 1 (Trademark) System 677. Ringer's Inj Lactated USP 500 ml Bag 685 may be NSN NDC 0409-7953-03. Kit—SBTM Tactical IV Starter Kit 681 may be NSN 6515-01-587-5717. Access—FAST 1 (Trademark) System 677 may be NSN 6515-01-536-9363.

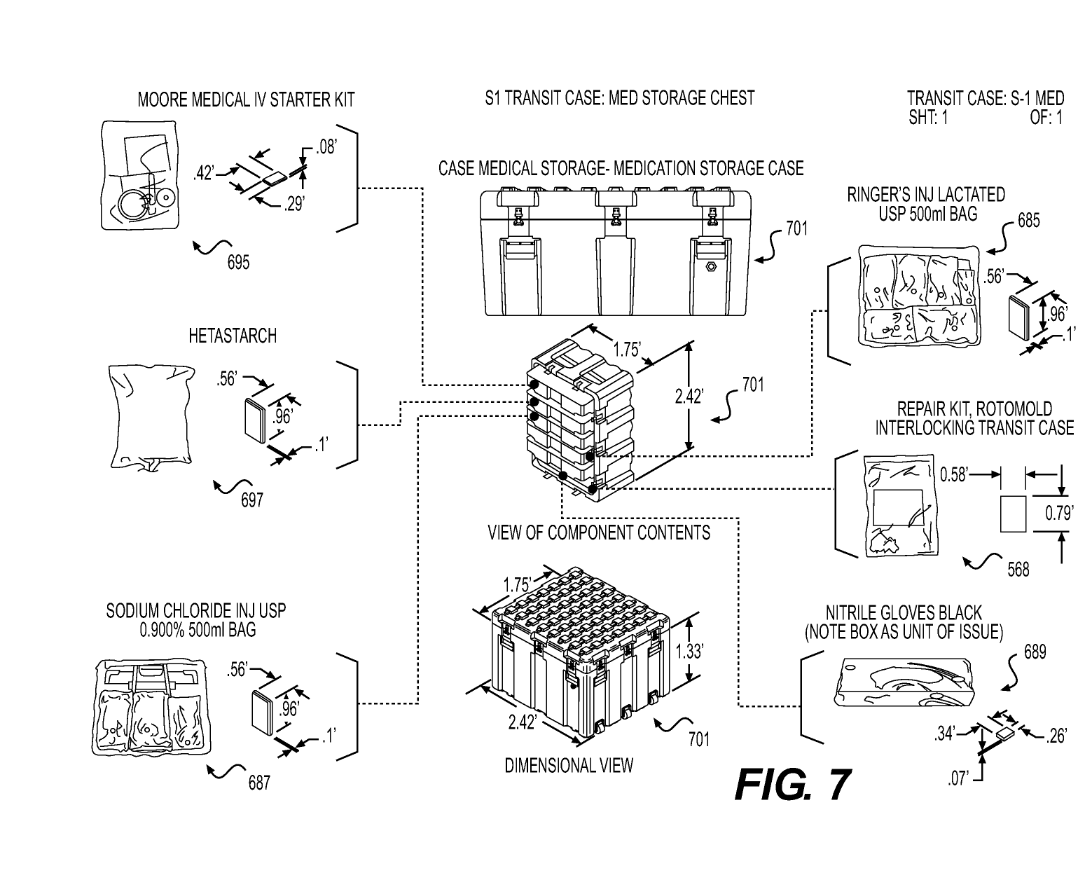

FIG. 7 illustrates the exemplary Med Storage Chest of the S-1 transit case 140 of the sustainment kit 106. Case Medical Storage—Medication Storage Chest 701 is shown containing Moore Medical IV Starter Kit 695, Hetastarch 697, Sodium Chloride Inj USP 0.900% 500 ml Bag 687, Ringer's Inj Lactated USP 500 ml Bag 685, Repair Kit, Rotomold Interlocking Transit Case 568, and Nitrile Gloves Black (note box as unit of issue) 689. Medical Storage—Medication Storage Chest 701 may be NSN 815-01-606-1885.

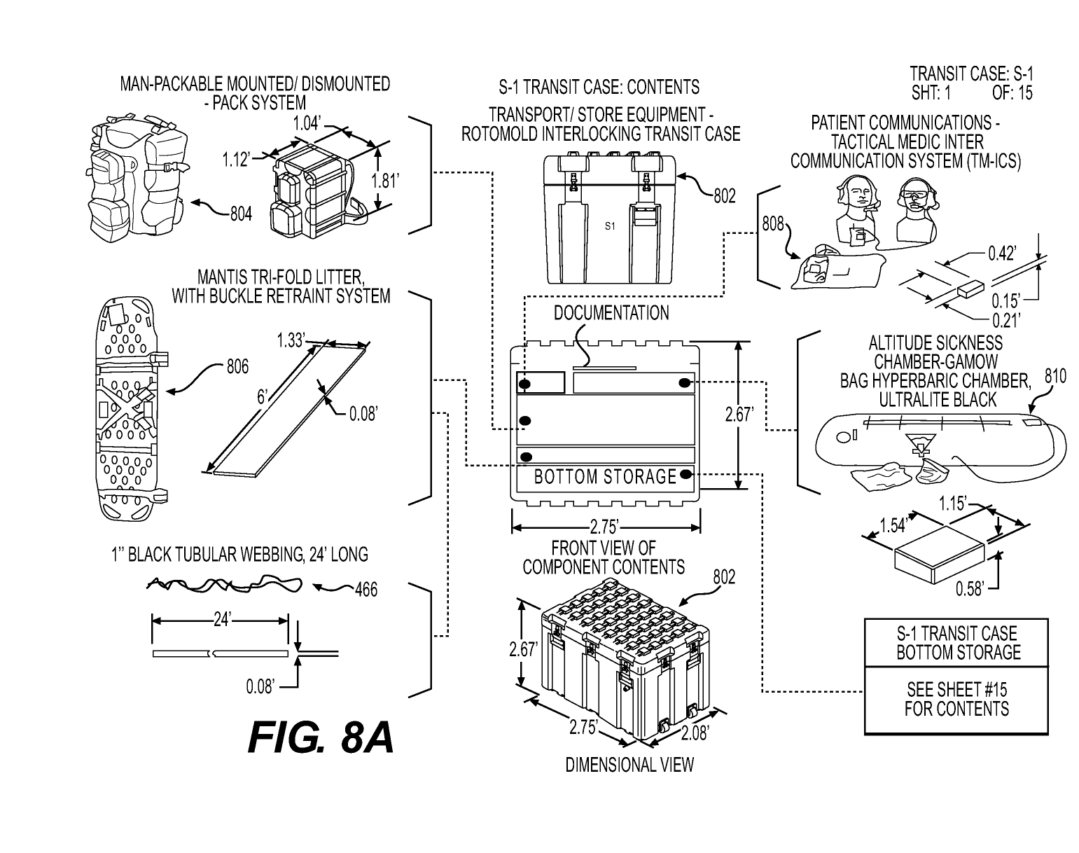

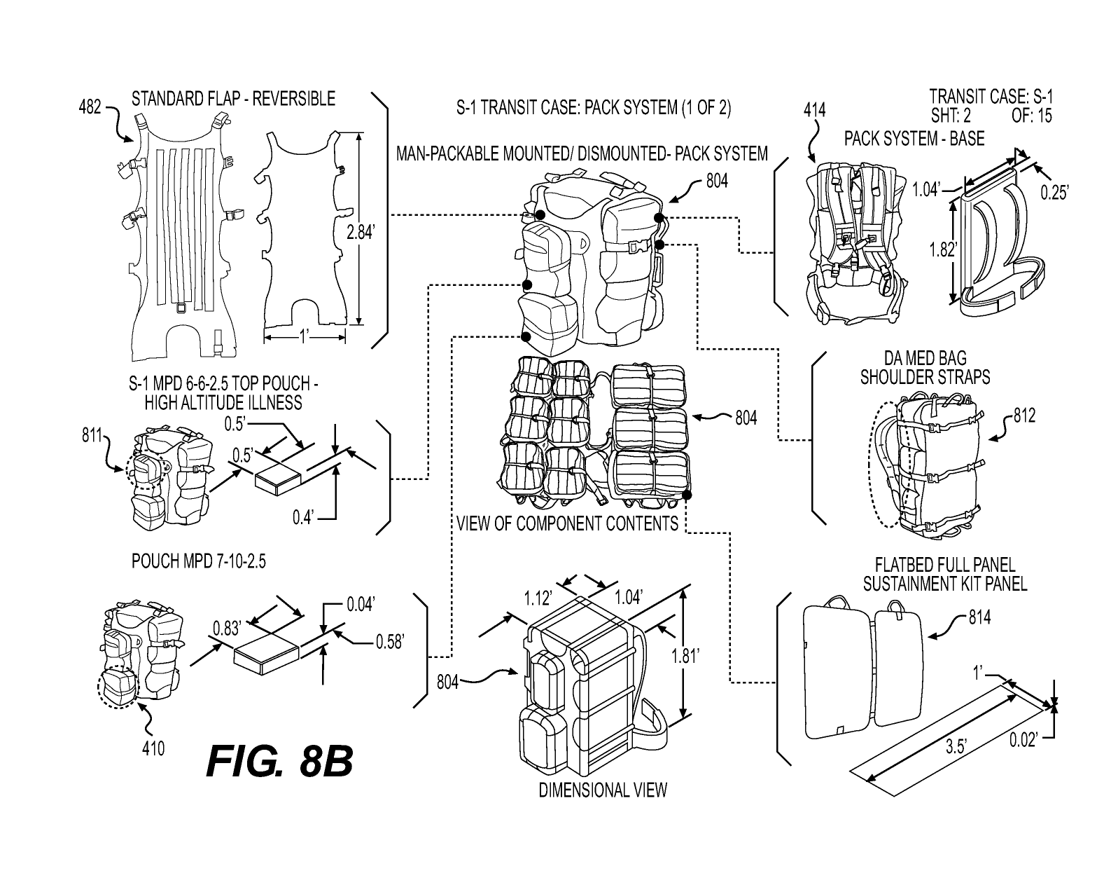

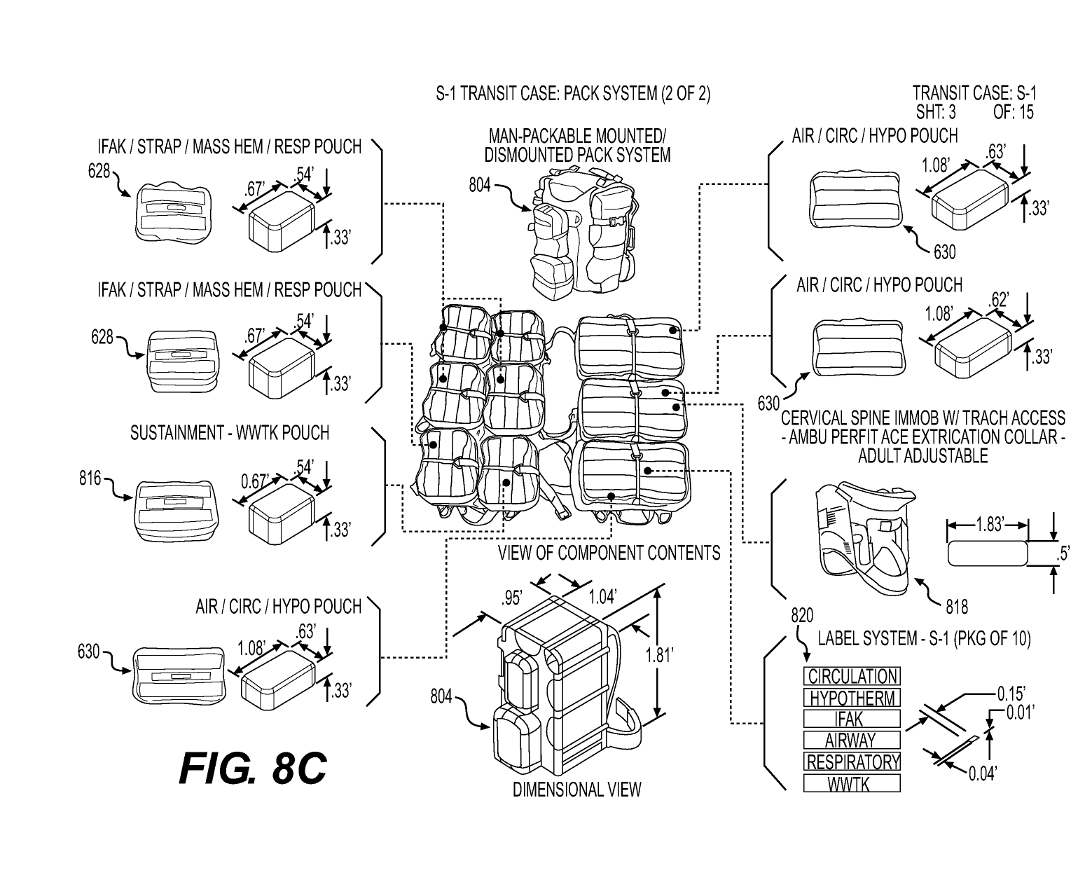

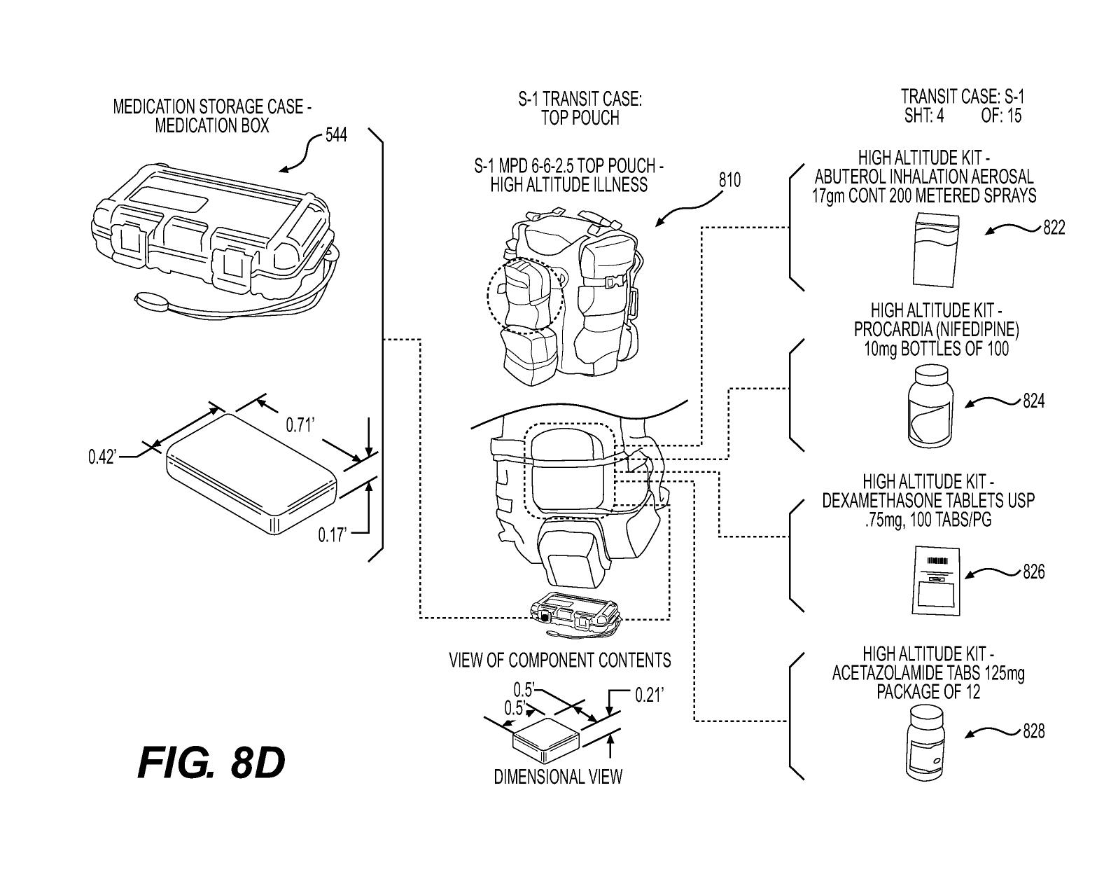

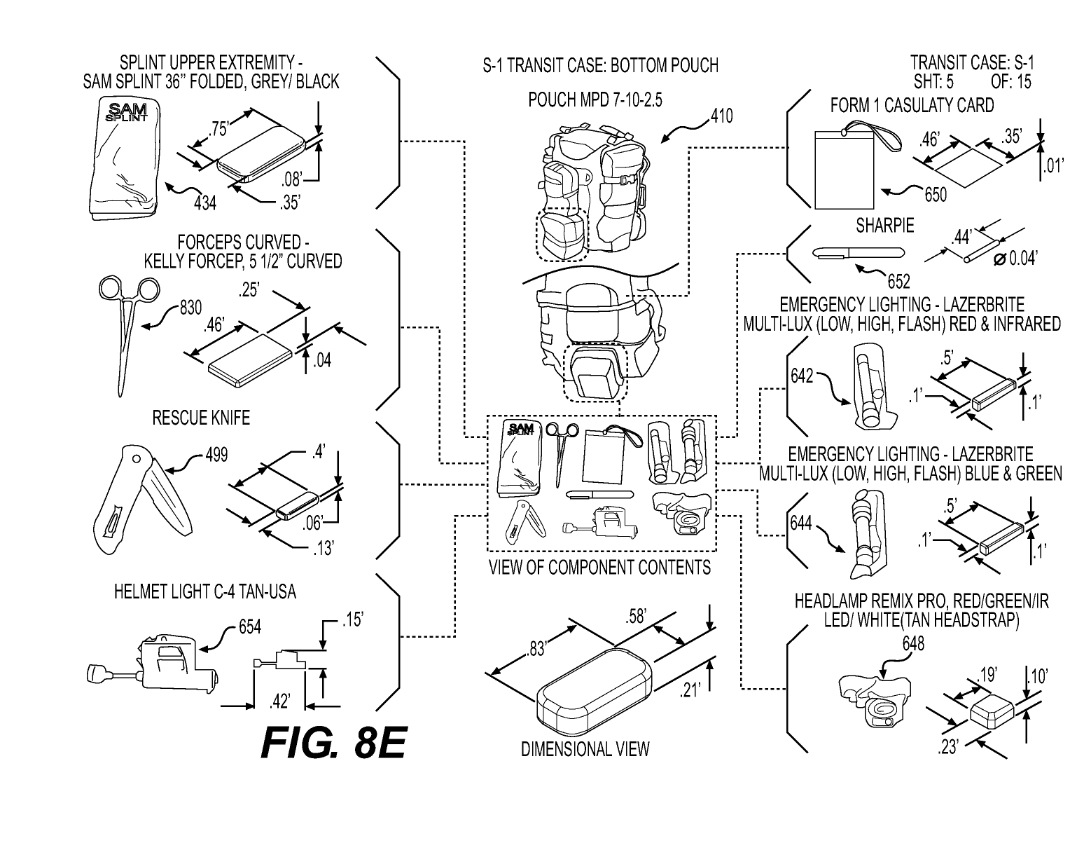

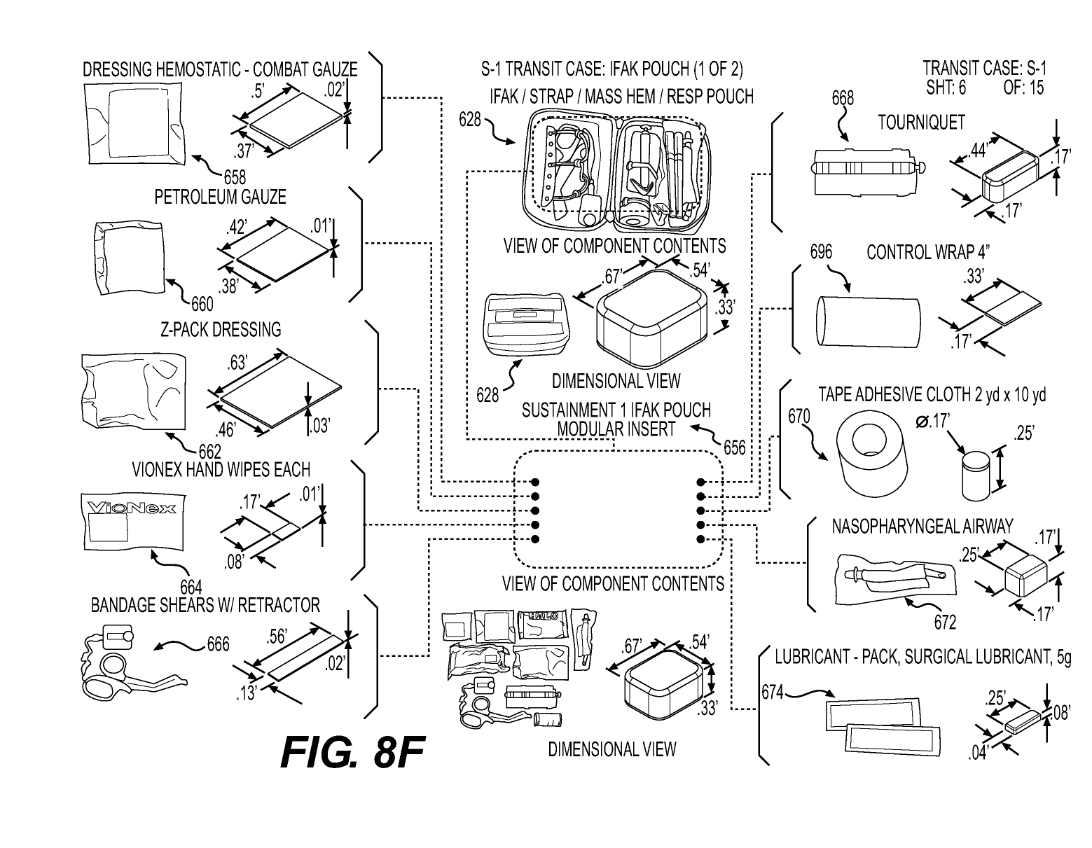

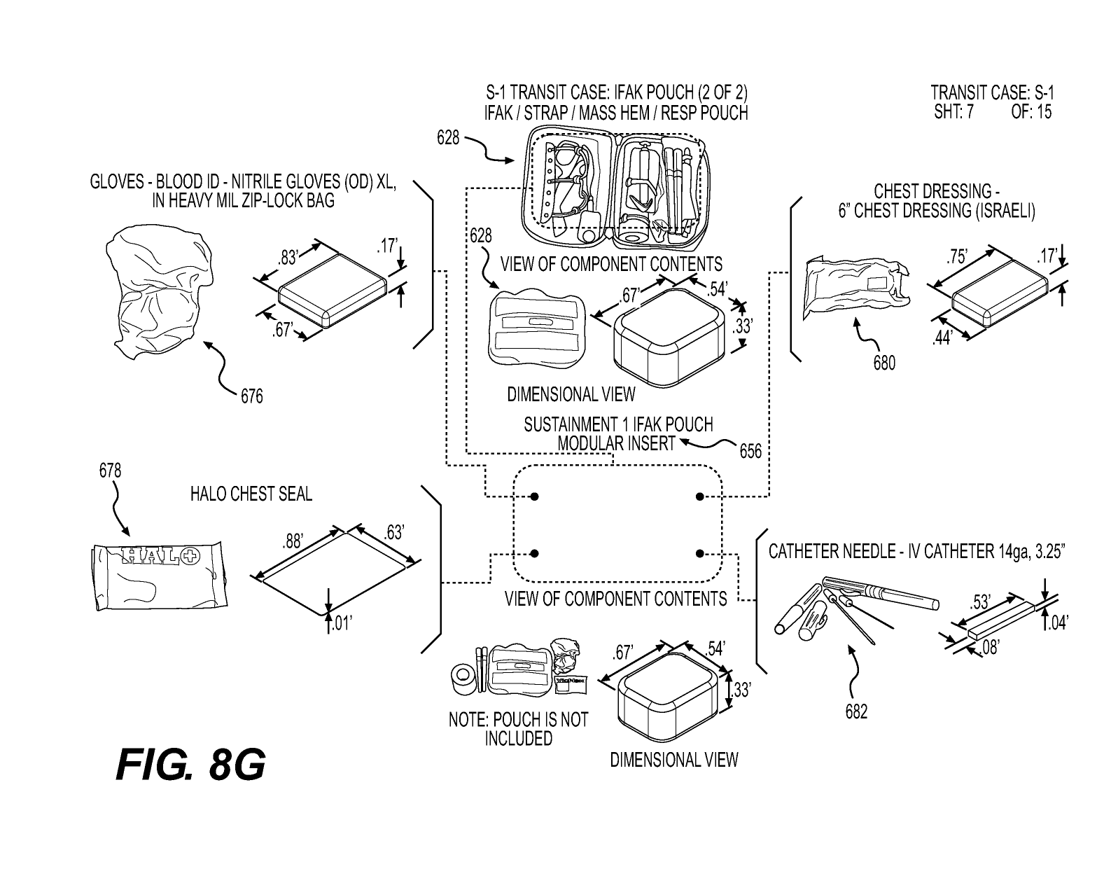

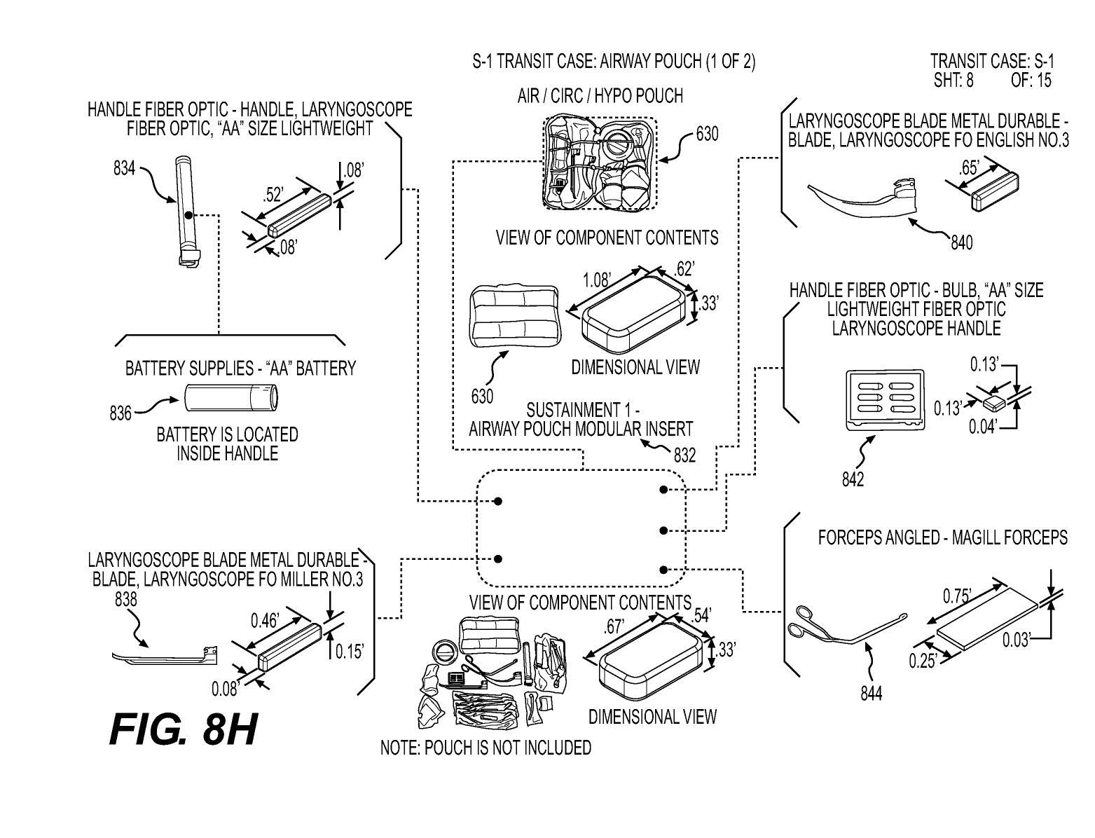

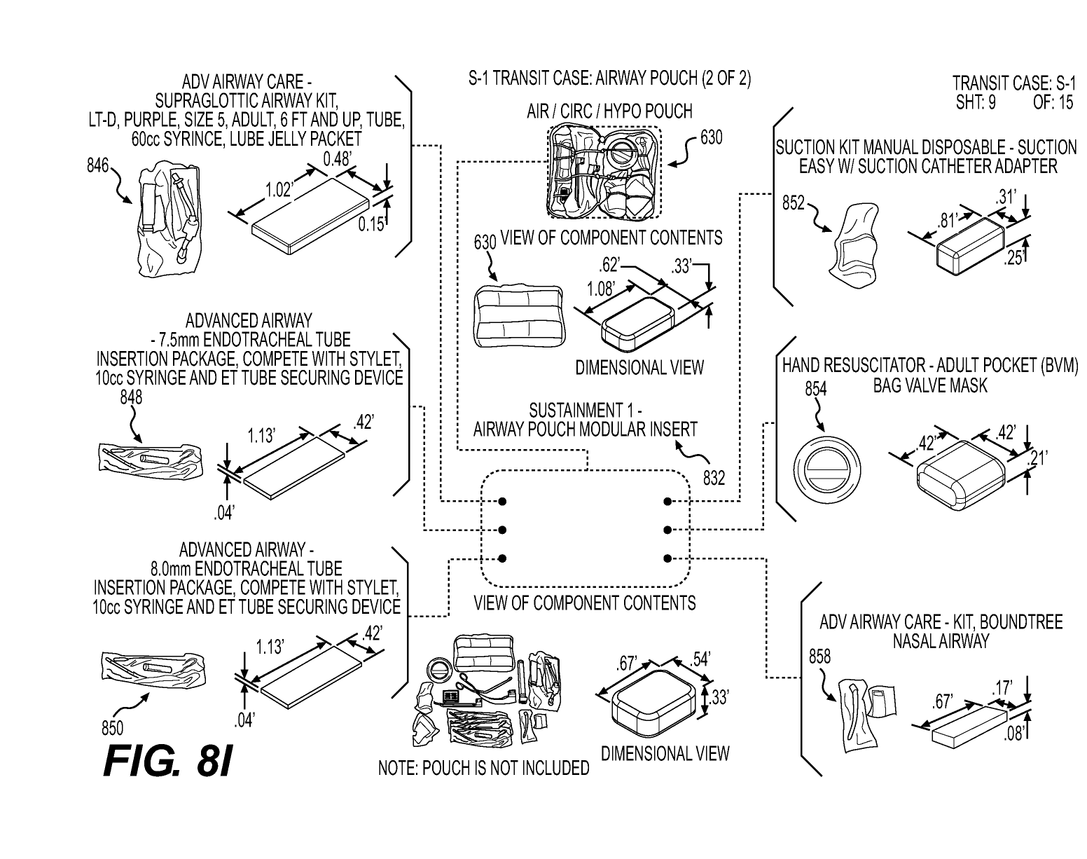

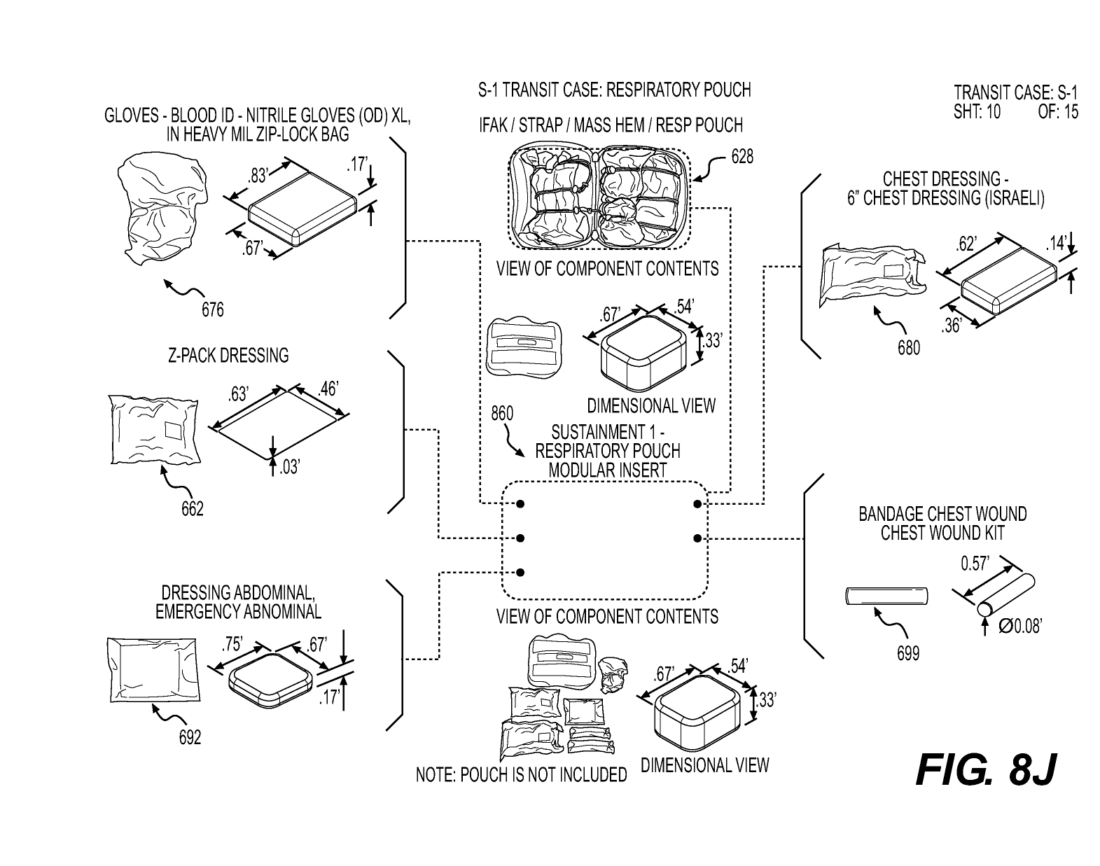

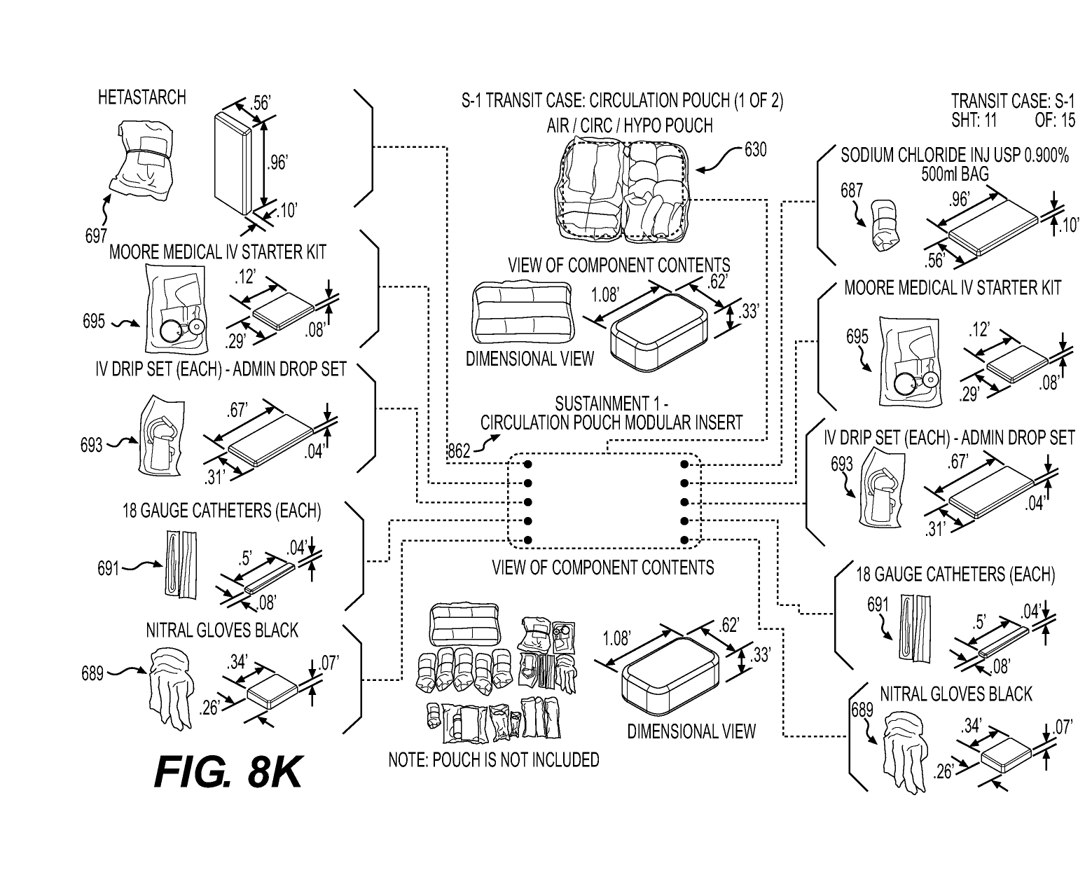

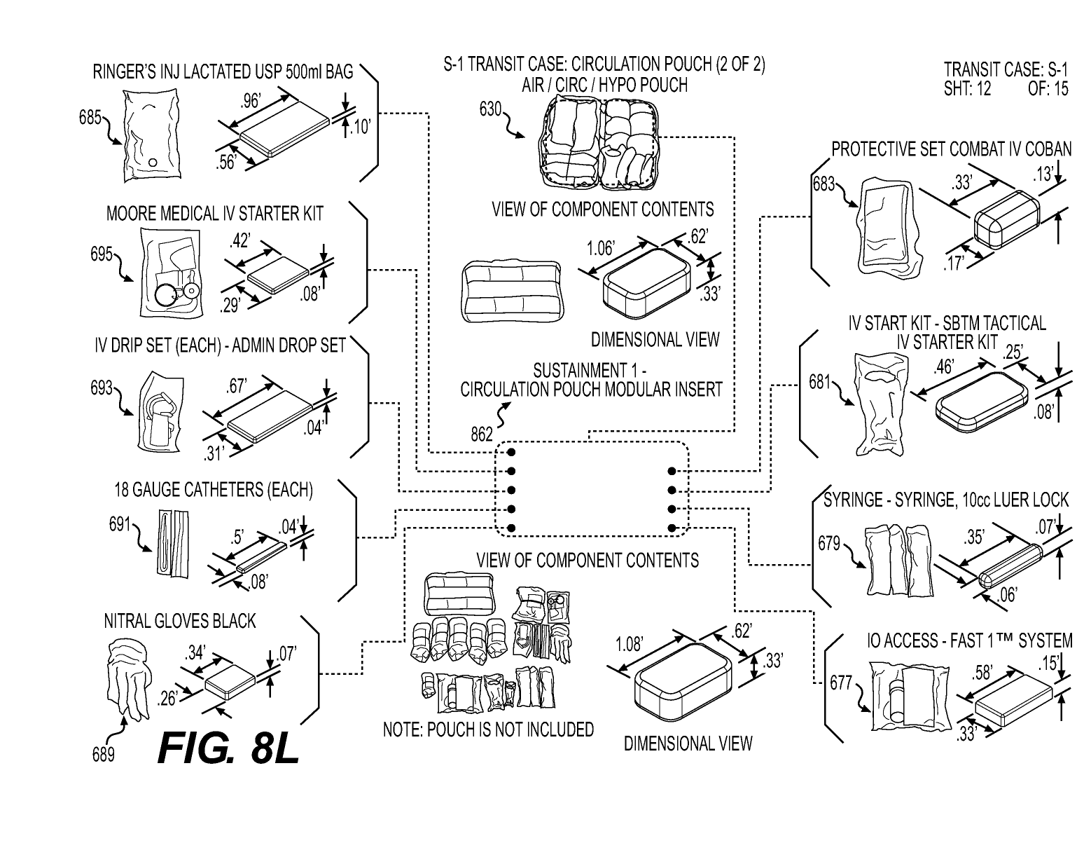

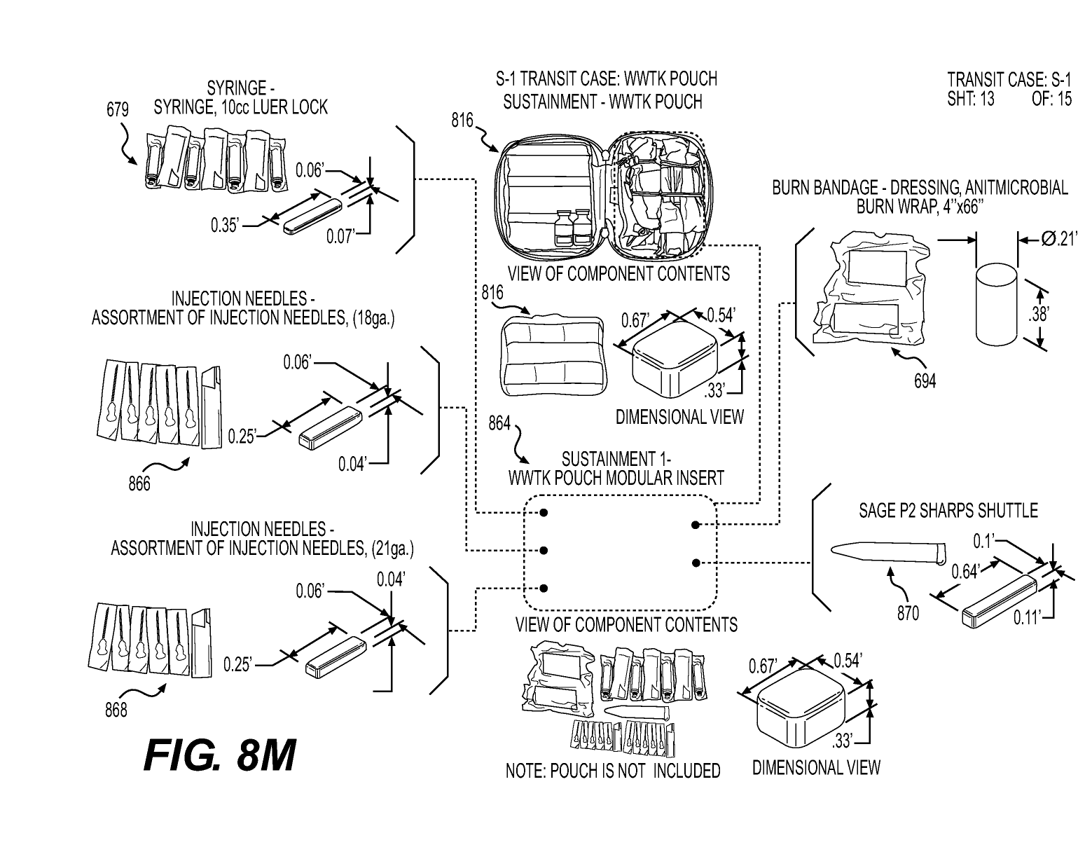

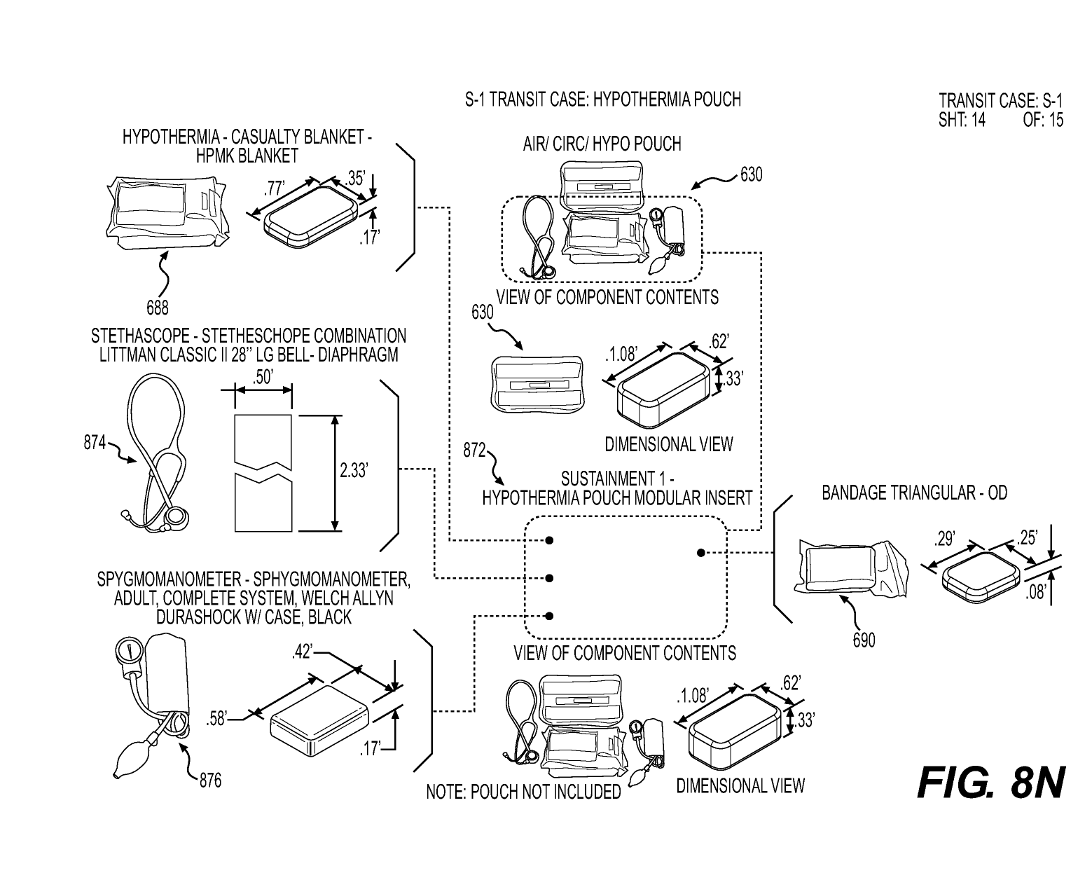

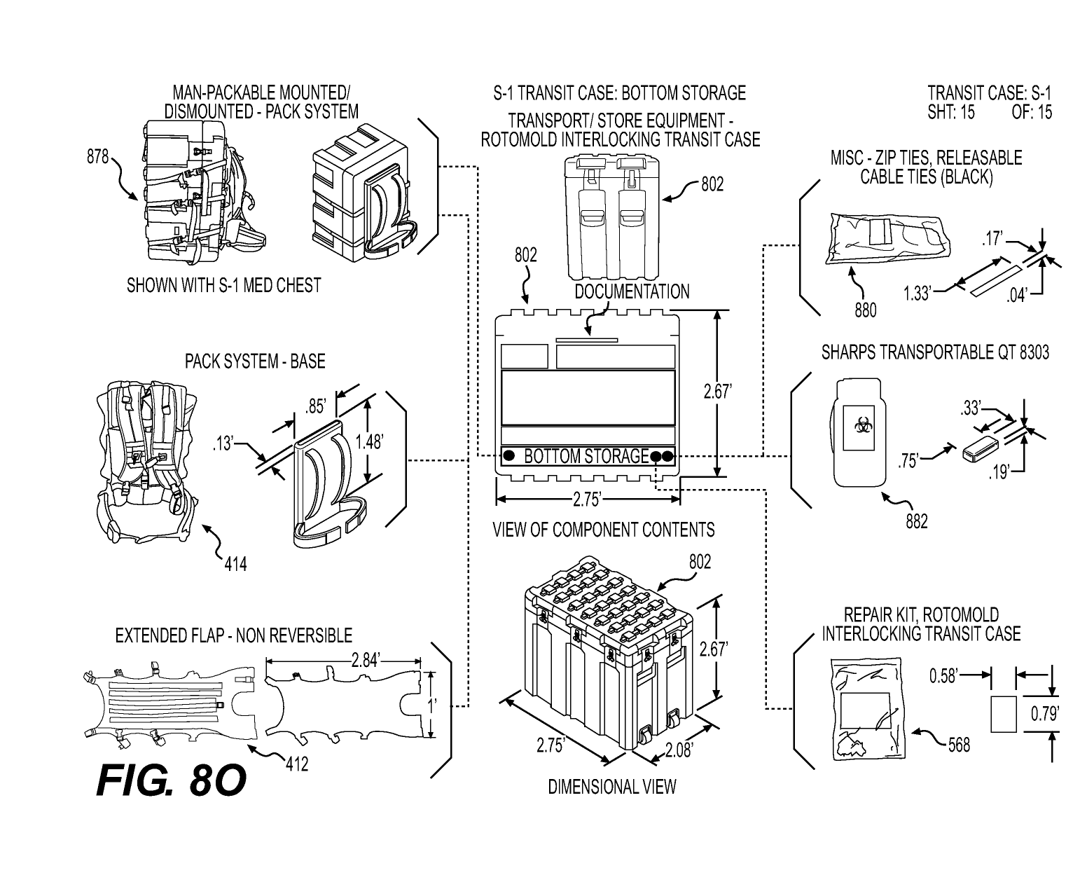

FIG. 8A illustrates the exemplary case contents of the S-1 transit case 140 of the sustainment kit 106. Transport/Store equipment—Rotomold Interlocking Transit Case 802 is shown containing Man-Packable Mounted/Dismounted—Pack System 804, Mantis Tri Fold Litter, with buckle restraint system 806, 1″ black tubular webbing, 24″ long 466, Patient Communications—Tactical Medic InterCommunicationSystem (TM-ICS) 808, and Altitude Sickness Chamber-Gamow Bag Hyperbaric Chamber, Ultralite Black 810. Transport/Store equipment—Rotomold Interlocking Transit Case 802 may be NSN 8145-01-606-1893. Man-Packable Mounted/Dismounted—Pack System 804 may be NSN 8465-01-607-0188, 0196, 0199 or 0201. Communications—Tactical Medic InterCommunicationSystem (TM-ICS) 808 may be NSN 5830-01-606-2282. Altitude Sickness Chamber-Gamow Bag Hyperbaric Chamber, Ultralite Black 810 may be NSN 6515-01-618-9774.

FIG. 8B depicts an exemplary pack system of the S-1 transit case 140. This is the first of two figures that depict this pack system, the second being FIG. 8C below. Man-Packable Mounted/Dismounted—Pack System 804 is shown containing Reversible Standard Flap 482, S-1 MPD 6-6-2.5 Top Pouch—High Altitude Illness 811, Pouch MPD 7-10-2.5 410, Pack System Base 414, DA (Direct Action) Med Bag Shoulder Straps 812 and Flatbed Full Panel Sustainment Kit Panel 814. As shown here and on FIG. 5C, these sustainment panels includes two sustainment panels, which may be side-by-side. This may have the benefit of including more components in a more compact area, and reducing total length as opposed to using a single sustainment panel. Dual panels (versus one) allows for additional customization when working in confined spaces. S-1 MPD 6-6-2.5 Top Pouch—High Altitude Illness 811 may be NSN 8465-01-607-0169, 0177, 0540 or 0553. DA Med Bag Shoulder Straps 812 may be NSN 8465-01-607-0611, 0613, 0622 or 0626. Flatbed Full Panel Sustainment Kit Panel 814 may be NSN 8465-01-607-0166, 0209, 0211 or 0214.

FIG. 8C illustrates the exemplary Pack System of the S-1 transit case 140. This is the second figure of 2 figures disclosing this pack system, the first being FIG. 8B. Man-Packable Mounted/Dismounted—Pack System 804 is shown containing IFAK/Strap/Mass Hem/Resp Pouch 628 (×2), Sustainment—WWTK (War Wound Therapy Kit) Pouch 816, Air/Circ/Hypo Pouch 630 (×3), Cervical Spine Immobilizer with Trach Access-AMBU Perfit Ace Extrication Collar—Adult Adjustable 818 and Label System—S-1 (PKG of 10) 820. Sustainment—WWTK Pouch 816 may be NSN 8465-01-607-0174, 0219, 0542 or 0554. Label System—S-1 (PKG of 10) 820 may be NSN 7690-01-607-0560.

FIG. 8D illustrates the exemplary top pouch of the S-1 transit case 140. S-1 MPD 6-6-2.5 Top Pouch—High Altitude Illness 810 is shown containing Medication Storage Case—Medication Box 544. Medication Storage Case—Medication Box 544 is shown containing High Altitude Kit—Albuterol Inhalation Aerosal 17GM Count 200 Metered Sprays 822, High Altitude Kit—Procardia (Nifedipine) 10 mg Bottles of 100 824, High Altitude Kit—Dexamethasone Tablets USP 0.75 mg, 100 Tabs/PG 826 and High Altitude Kit—Acetazolamide Tabs 125 mg Package of 12 828. High Altitude Kit—Albuterol Inhalation Aerosal 17GM Count 200 Metered Sprays 822 may be NSN NDC 59310057920. High Altitude Kit—Dexamethasone Tablets USP 0.75 mg, 100 Tabs/PG 826 may be NSN NDC 54817625. High Altitude Kit—Acetazolamide Tabs 125 mg Package of 12 828 may be NSN NDC 51672402301.

FIG. 8E illustrates the exemplary bottom pouch of the S-1 transit case 140. Pouch MPD 7-10-2.5 410 is shown containing Splint Upper Extremity—Sam Splint 36″ Folded, Grey/Black 434, Forceps Curved—Kelly Forcep, 5½″ Curved 830, Rescue Knife 499, Helmet Light C-4 Tan-USA 654, Form 1 Casualty Card 650, Sharpie 652, Emergency Lighting—LazerBrite Multi-Lux (low, high, flash)—Red & Infrared 642, Emergency Lighting—LazerBrite Multi-Lux (low, high, flash)—Blue & Green 644, and Headlamp Remix Pro, Red/Green/IR Led/White (Tan Headstrap) 648.

FIG. 8F illustrates an exemplary IFAK Pouch of the S-1 transit case 140. This is the first of two figures showing this pouch, the second being FIG. 8G below. IFAK/Strap/Mass Hem/Resp Pouch 628 is shown containing Sustainment 1 IFAK Pouch Modular Insert 656. Sustainment 1 IFAK Pouch Modular Insert 656 is shown containing Dressing Hemostatic—Combat Gauze 658, Petroleum Glaze 660, Z-Pack Dressing 662, Vionex Hand Wipes Each 664, Bandage Shears with Retractor 666, Tourniquet 668, Control Wrap 4″ 696, Tape Adhesive Cloth 2 in×10 yd 670, Nasopharyngeal Airway 672 and Lubricant—Pack, Surgical Lubricant, 5g 674.

FIG. 8G illustrates an exemplary IFAK Pouch of the S-1 transit case 140. This is the second of two figures illustrating this pack, the first being FIG. 8F. IFAK/Strap/Mass Hem/Resp Pouch 628 is shown containing Sustainment 1 IFAK Pouch Modular Insert 656. Sustainment 1 IFAK Pouch Modular Insert 656 is shown containing Gloves—Blood ID—Nitrile Gloves (OD), XL, in heavy Mil zip-lock bag 676, Halo Chest Seal 678, Chest Dressing—6″ Chest Dressing (Isreali) 680 and Catheter Needle—IV Catheter 14GA, 3.25″ 682.

FIG. 8H depicts an exemplary Airway Pouch of the S-1 transit case 140. This is the first of two figures showing this pouch, the second being FIG. 5G below. Air/Circ/Hypo Pouch 630 is shown containing Sustainment 1—Airway Pouch Modular Insert 832. Sustainment 1—Airway Pouch Modular Insert 832 is shown containing Handle Fiber Optic—Handle, Laryngoscope Fiber Optic, “AA” Size Lightweight 834, Battery Supplies—“AA” Battery 836, Laryngoscope Blade Metal Durable—Blade, Laryngoscope FO Miller No. 3 838, Laryngoscope Blade Metal Durable—Blade, Laryngoscope FO English No. 3 840, Handle Fiber Optic—Bulb, “AA” Size lightweight fiber optic laryngoscope handle 842 and Forceps Angled—Magill Forceps 844. Sustainment 1—Airway Pouch Modular Insert 832 may be NSN 6545-01-607-7872.

FIG. 8I illustrates the exemplary Airway Pouch of the S-1 transit case 140. This is the second of two figures illustrating this pouch, the first being FIG. 5H. Air/Circ/Hypo Pouch 630 is shown containing Sustainment 1—Airway Pouch Modular Insert 832. Sustainment 1—Airway Pouch Modular Insert 832 is shown containing Adv Airway Care—Supragloitic Airway Kit, LT-D, purple, size 5, adult, 6 ft and up, tube, 60 cc syringe, lube jelly packed 846, Advanced Airway—7.5 MM Endotracheal tube insertion package, complete with stylet, 10 CC syringe and ET Tube (Endotracheal tube) securing device 848, Advanced Airway—8.0 MM Endotracheal tube insertion package, complete with stylet, 10 CC syringe and ET Rube securing device 850, Suction Kit Manual Disposable—Suction Easy with Suction Catheter Adapter 852, Hand Resuscitator—Adult Pocket (BVM) Bag Valve Mask 854 and Adv Airway Care—Kit, Boundtree Nasal Airway 858. Adv Airway Care—Supragloitic Airway Kit, LT-D, purple, size 5, adult, 6 ft and up, tube, 60 cc syringe, lube jelly packed 846 may be NSN 6515-01-515-0161. Suction Kit Manual Disposable—Suction Easy with Suction Catheter Adapter 852 may be NSN 6515-01-516-3120. Hand Resuscitator—Adult Pocket (BVM) Bag Valve Mask 854 may be NSN 6515-01-593-4841. and Adv Airway Care—Kit, Boundtree Nasal Airway 858 may be NSN 6515-01-518-8597.

FIG. 8J illustrates an exemplary Respiratory Pouch of the S-1 transit case 140. IFAK/Strap/Mass Hem/Resp Pouch 628 is shown containing Sustainment 1—Respiratory Pouch Modular Insert 860. Sustainment 1—Respiratory Pouch Modular Insert 860 is shown containing Gloves—Blood ID—Nitrile Gloves (OD), XL, In heavy mil zip-lock bag 676, Z-Pack dressing 662, Dressing Abdominal, Emergency Abdominal 692, Chest Dressing—6″ Chest Dressing (Israeli) 680 and Bandage Chest wound Chest wound kit 699. Sustainment 1—Respiratory Pouch Modular Insert 860 may be NSN 6545-01-607-7903.

FIG. 8K illustrates an exemplary Circulation Pouch of the S-1 transit case 140. This is the first of two figures showing this circulation pouch, the second being FIG. 8L below. Air/Circ/Hypo (Airway, Circulation, Hypothermia) Pouch 630 is shown containing Sustainment 1—Circulation Pouch Modular Insert 862. Sustainment 1—Circulation Pouch Modular Insert 862 is shown containing Hetastarch 697, Moore Medical IV Starter Kit 695 (×2), IV Drip Set (each)—Admin Drop Set 693 (×2), 18 Gauge Catheters 691 (×2), Nitrile Gloves Black 689 (×2) and Sodium Chloride Inj USP 0.900% 500 ml Bag 687. Sustainment 1—Circulation Pouch Modular Insert 862 may be NSN 6545-01-607-7938.

FIG. 8L illustrates the exemplary Circulation Pouch of the S-1 transit case 140. This is the second of two figures illustrating this circulation pouch, the first being FIG. 8K above. Air/Circ/Hypo Pouch 630 is shown containing Sustainment 1—Circulation Pouch Modular Insert 862. Sustainment 1—Circulation Pouch Modular Insert 862 is shown containing Ringer's Inj Lactated USP 500 ml Bag 685, Moore Medical IV Starter Kit 695, IV Drip Set (each)—Admin Drop Set 693, 18 Gauge Catheters (Each) 691, Nitrile Gloves Black 689, Protective Set Combat IV Coban 683, IV Start Kit—SBTM Tactical IV Starter Kit 681, Syringe—Syringe, 10 CC Luer Lock 679 and IO Access—Fast 1 (Trademark) System 677.

FIG. 8M illustrates the exemplary WWTK Pouch of the S-1 transit case 140. Sustainment—WWTK Pouch 816 is shown containing Sustainment 1—WWTK Pouch Modular Insert 864. Sustainment 1—WWTK Pouch Modular Insert 864 is shown containing Syringe—Syringe, 10 CC Luer Lock 679, Injection Needles—Assortment of injection needles, (18GA) 866, Injection Needles—Assortment of Injection Needles, (21GA) 868, Burn Bandage—Dressing, Antimicrobial Burn Wrap, 4″×66″ 694, and Sage P2 Sharps Shuttle 870. Sustainment 1—WWTK Pouch Modular Insert 864 may be NSN 6545-01-607-7970.

FIG. 8N illustrates an exemplary Hypothermia Pouch of the S-1 transit case 140. Air/Circ/Hypo Pouch 630 is shown containing Sustainment 1—Hypothermia Pouch Modular Insert 872. Sustainment 1—Hypothermia Pouch Modular Insert 872 is shown containing Hypothermia—Casualty Blanket—HPMK Blanket 688, Stethescope—Stethoscope Combination Littman Classic II 28″ LG Bell-Diaphragm 874, Sphygmomanometer—Sphygmomanometer, Adult, Complete System, Welch Allyn Durashock WITHCase, Black 876 and Bandage Triangular—OD 690. Sustainment 1—Hypothermia Pouch Modular Insert 872 may be NSN 6545-01-607-8037. Stethescope—Stethoscope Combination Littman Classic II 28″ LG Bell-Diaphragm 874 may be NSN 6515-01-469-8665.

FIG. 8O illustrates the exemplary Bottom Storage of the S-1 transit case 140. Transport/Store Equipment—Rotomold Interlocking Transit Case 802 is shown containing Man-Packable Mounted/Dismounted—Pack System 878, Pack System Base 414, Extended Flap—Non Reversible 412, Zip Ties, Releasable Cable Ties (Black) 880, Sharps Transportable QT 8303 882 and Repair Kit, Rotomold Interlocking Transit Case 568. Man-Packable Mounted/Dismounted—Pack System 878 may be NSN 8465-01-607-0191, 0194, 0198 or 0205. Zip Ties, Releasable Cable Ties (Black) 880 may be NSN 5975-01-608-3692.

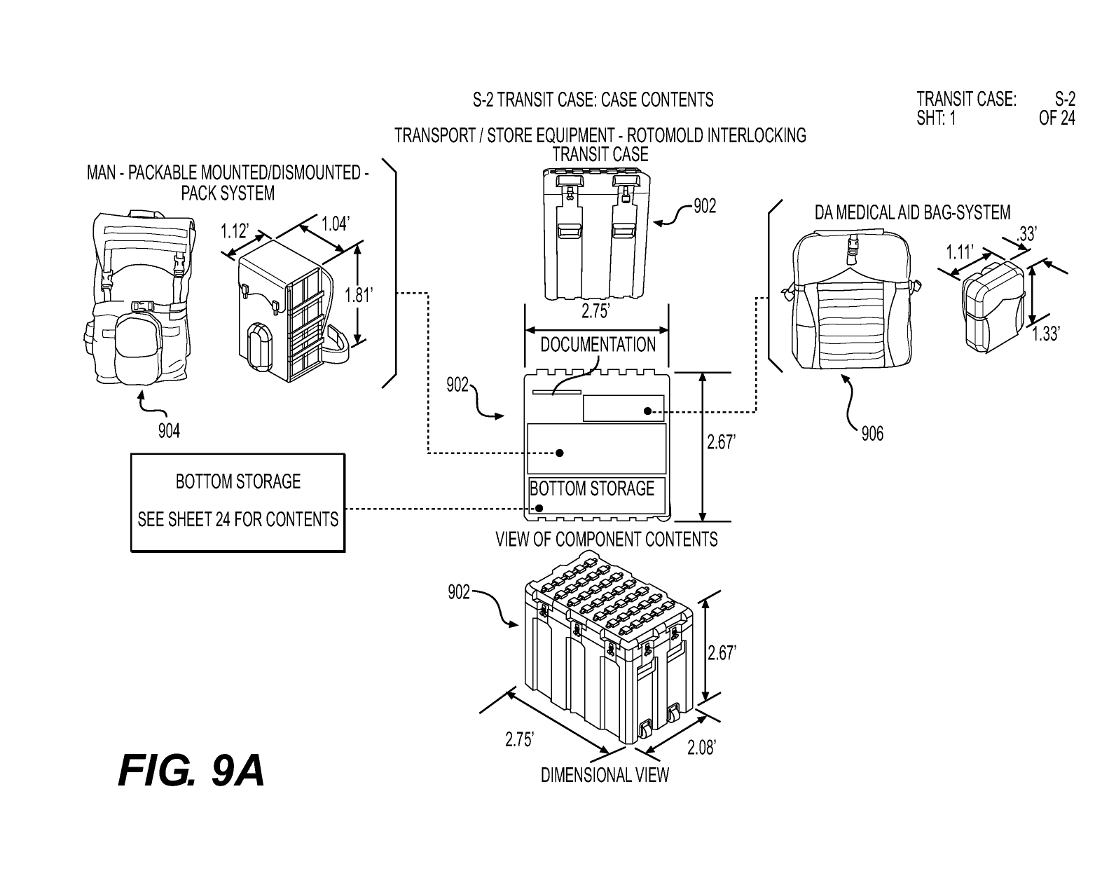

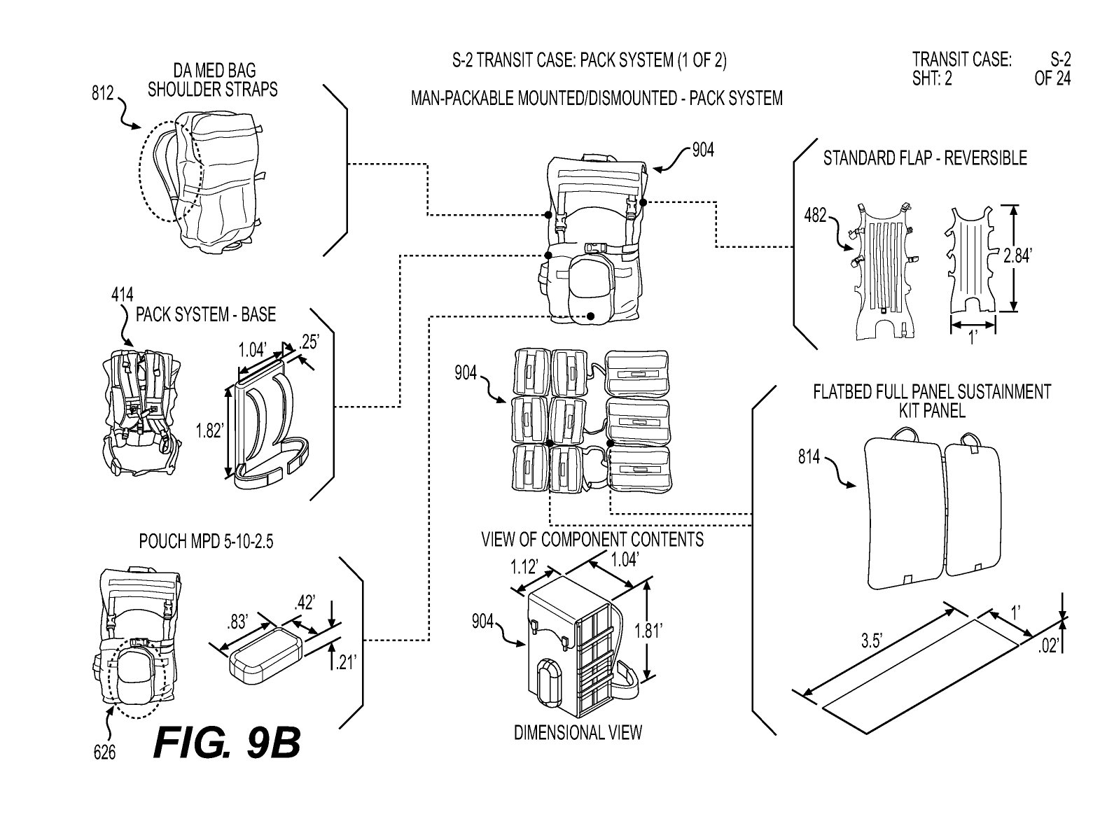

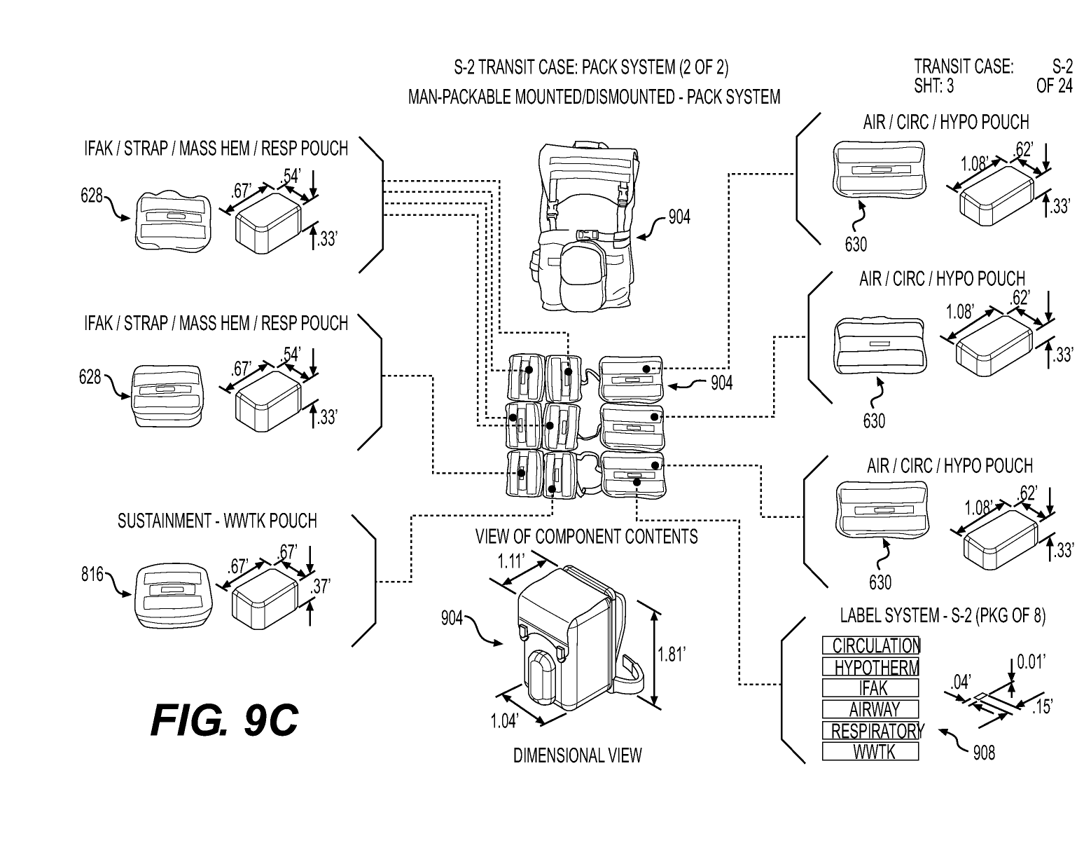

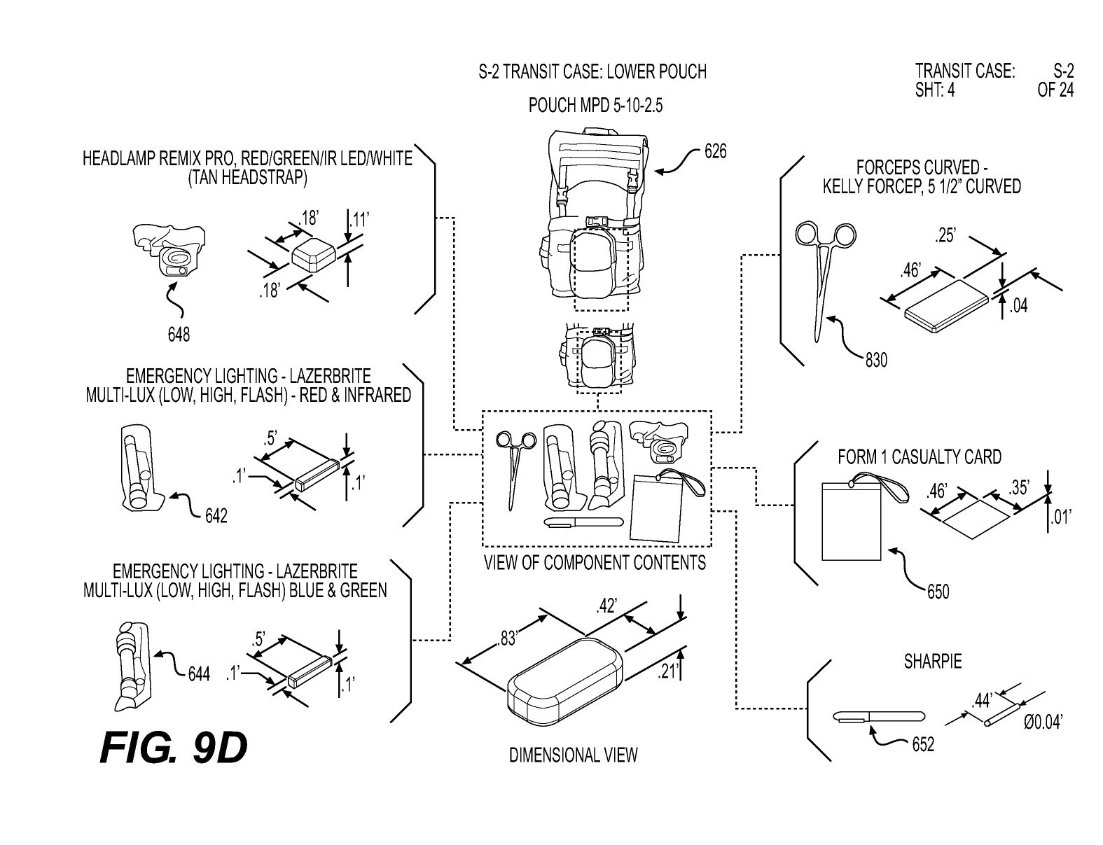

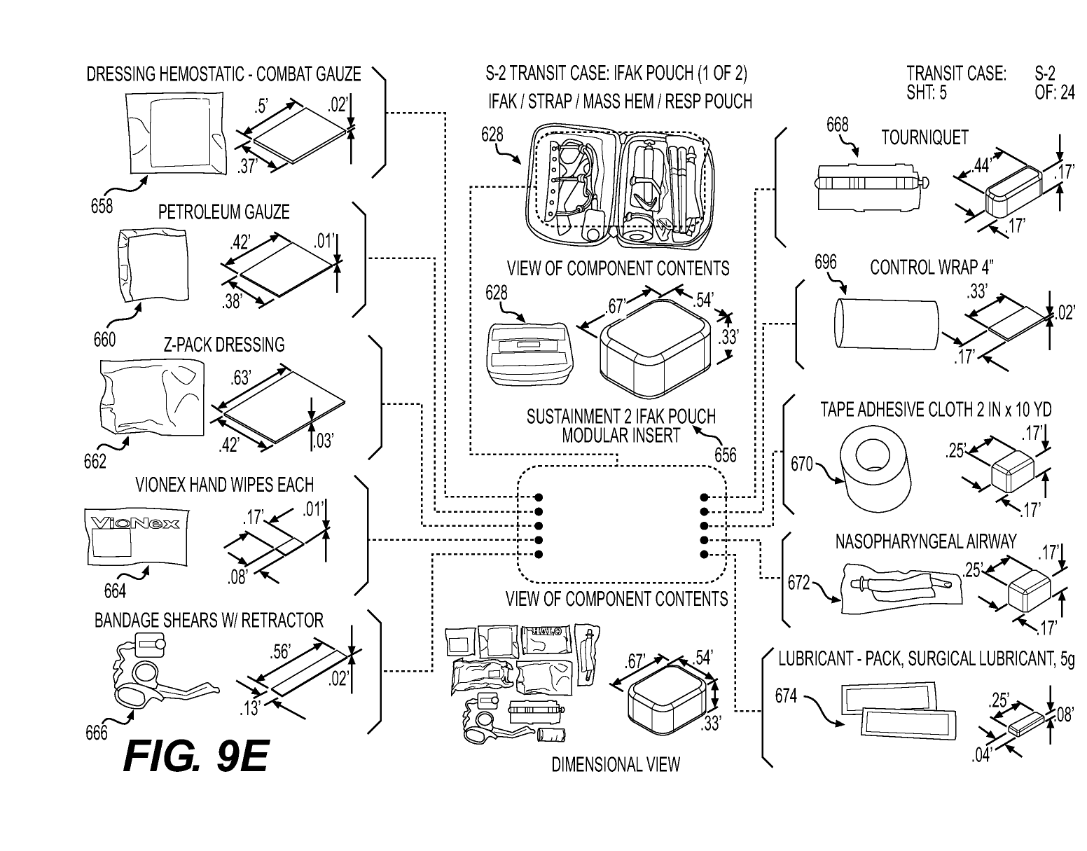

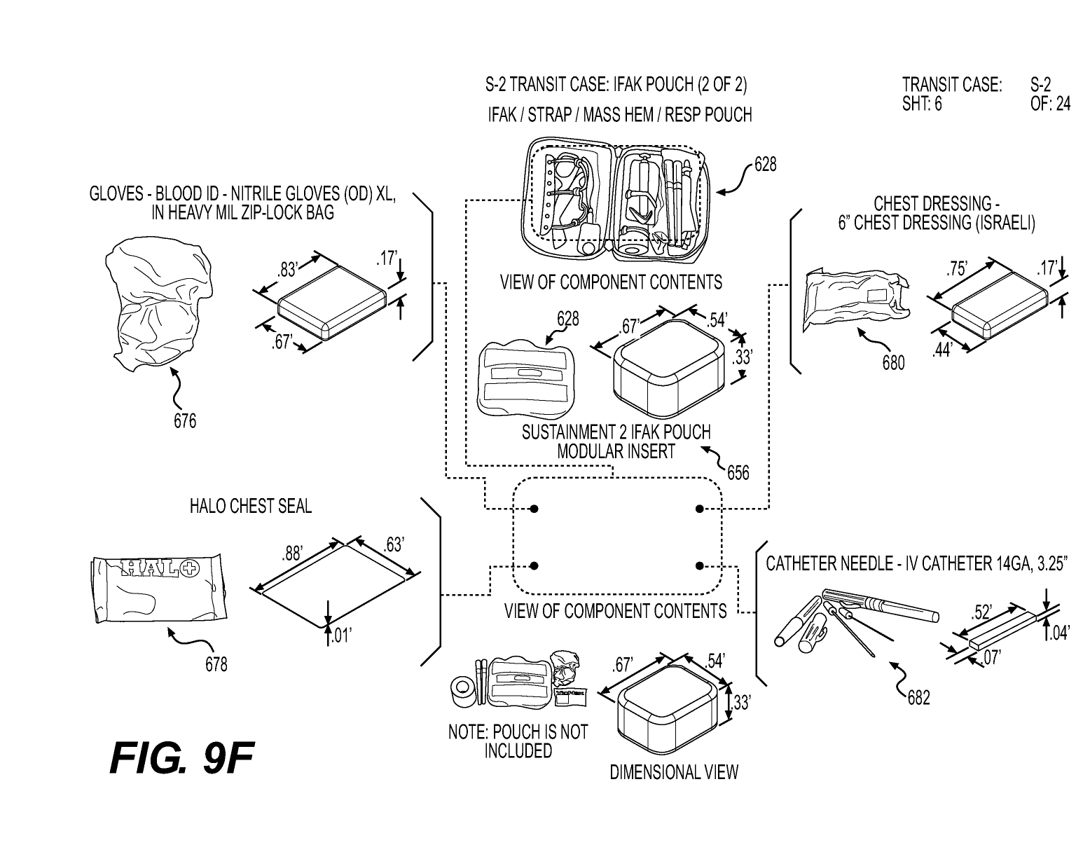

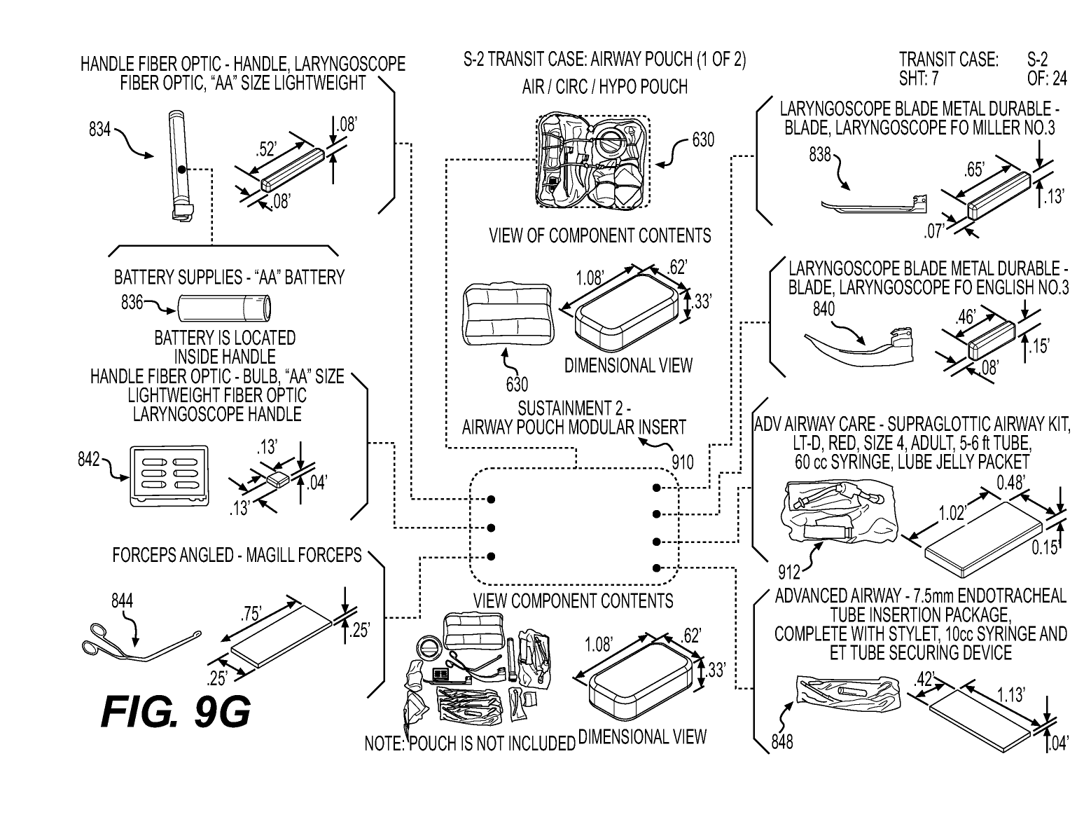

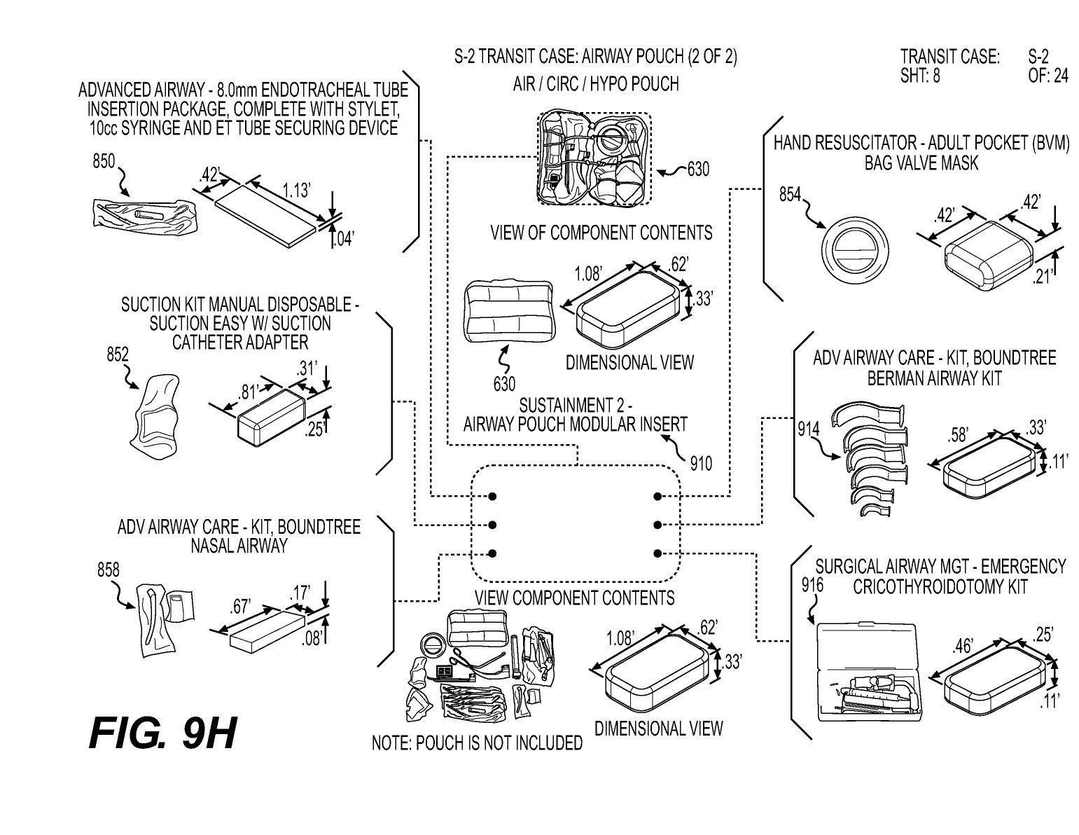

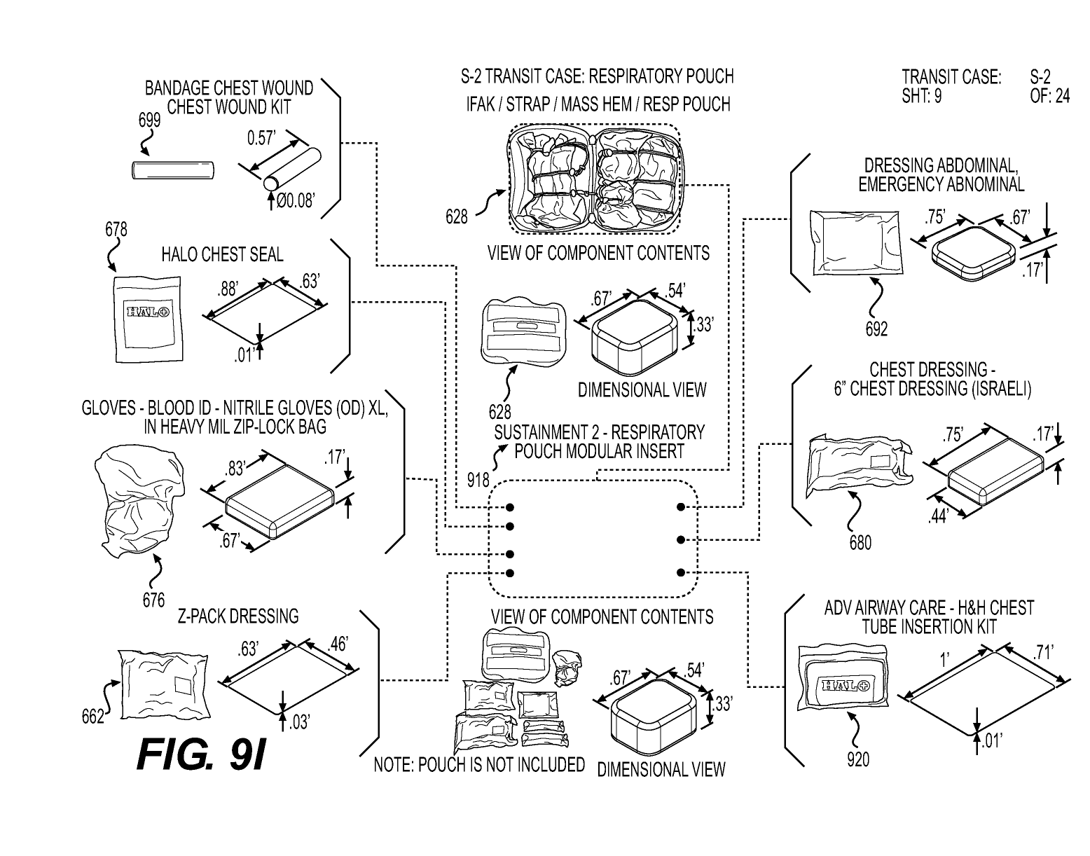

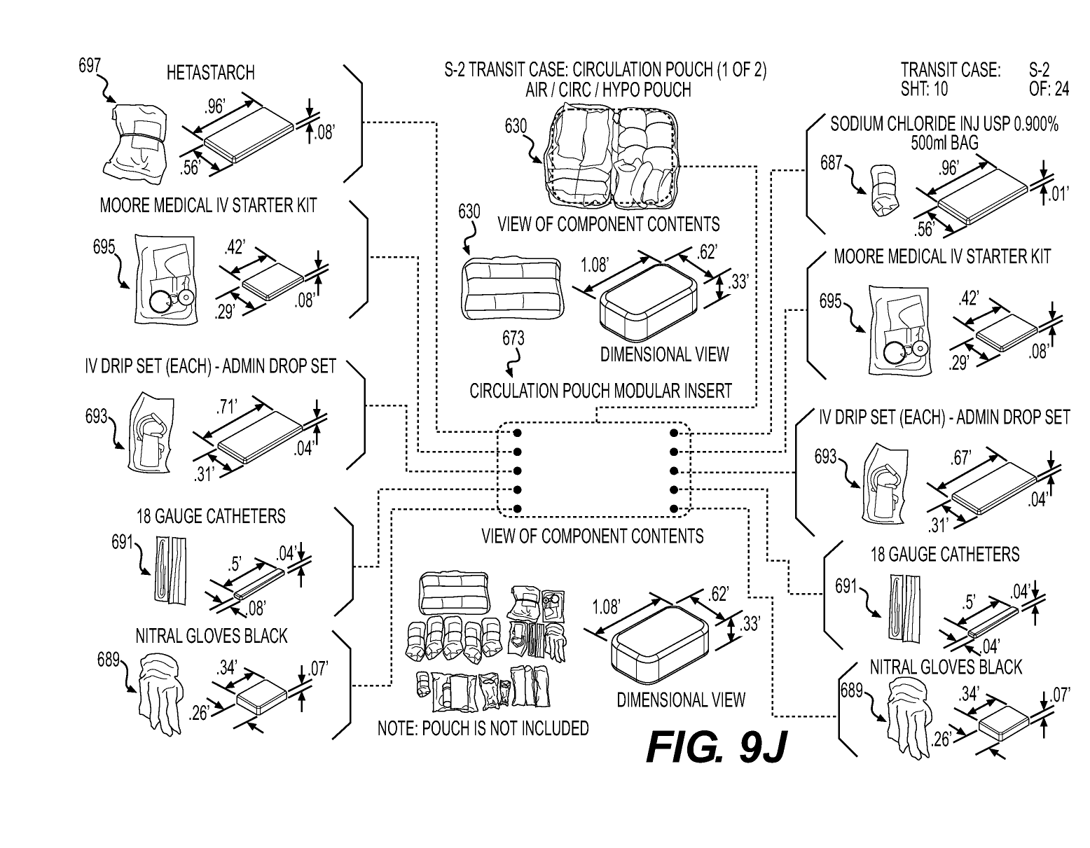

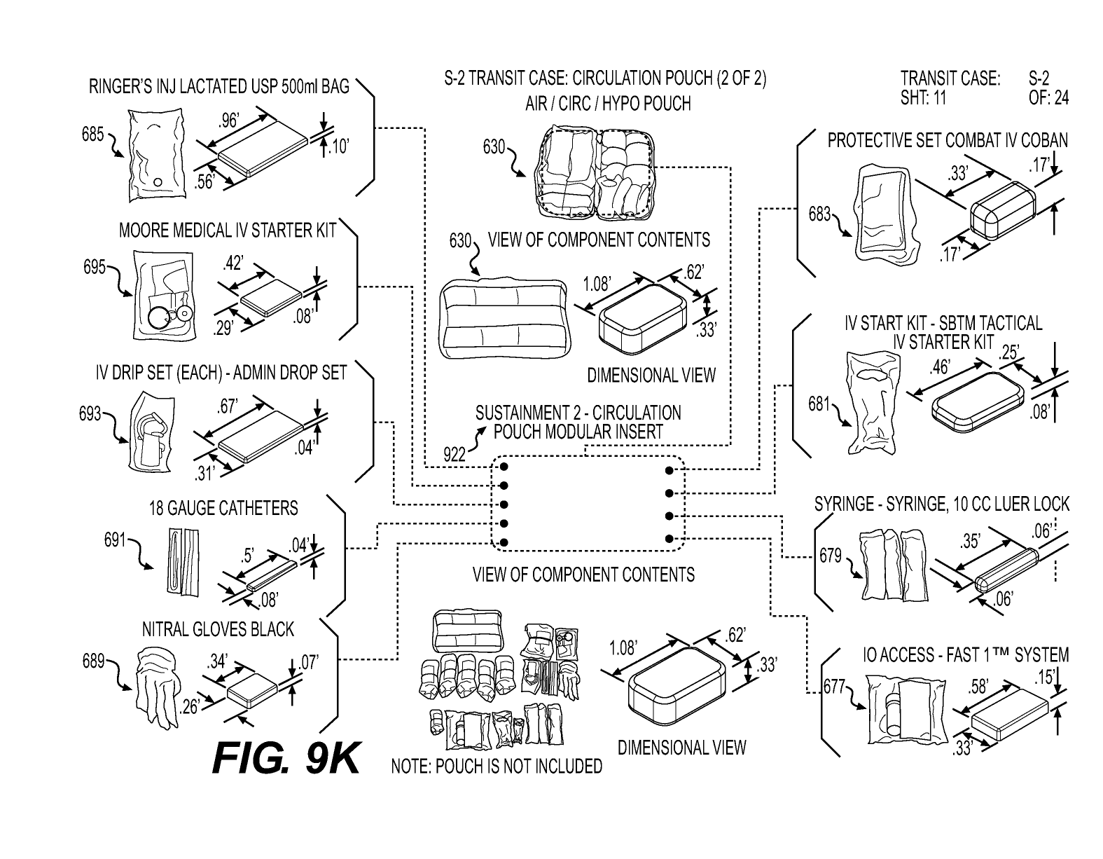

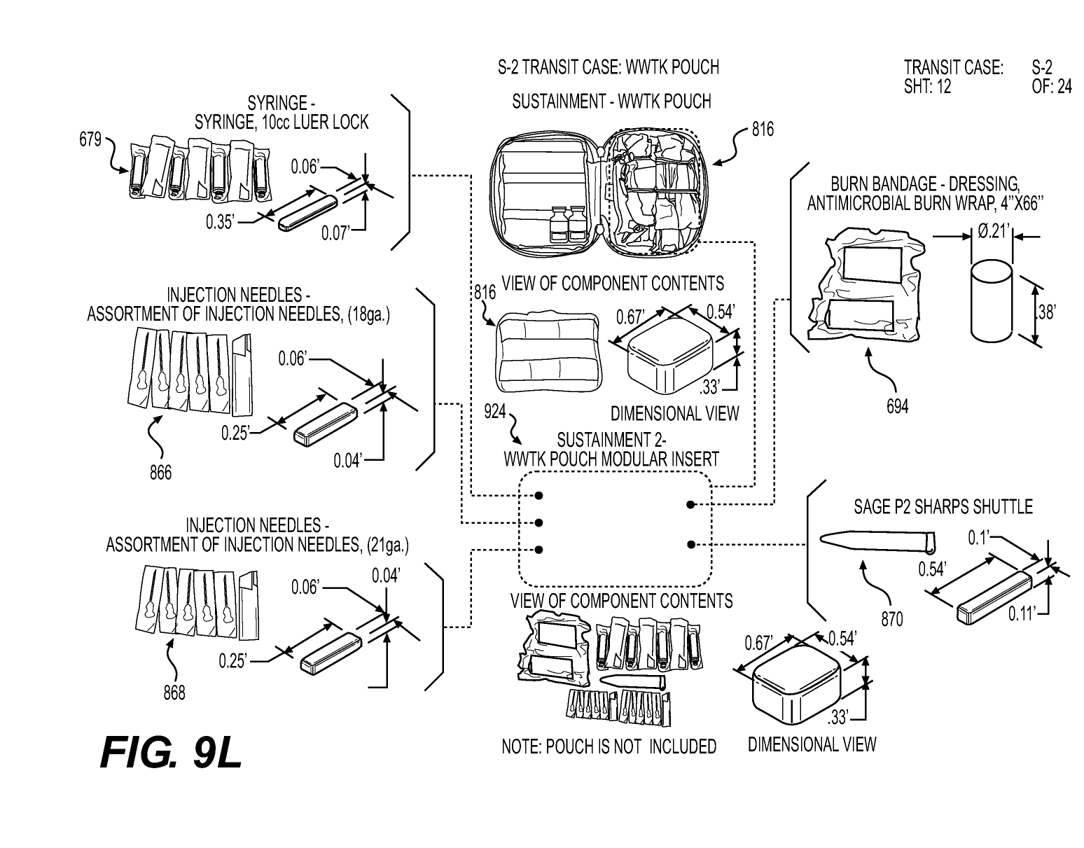

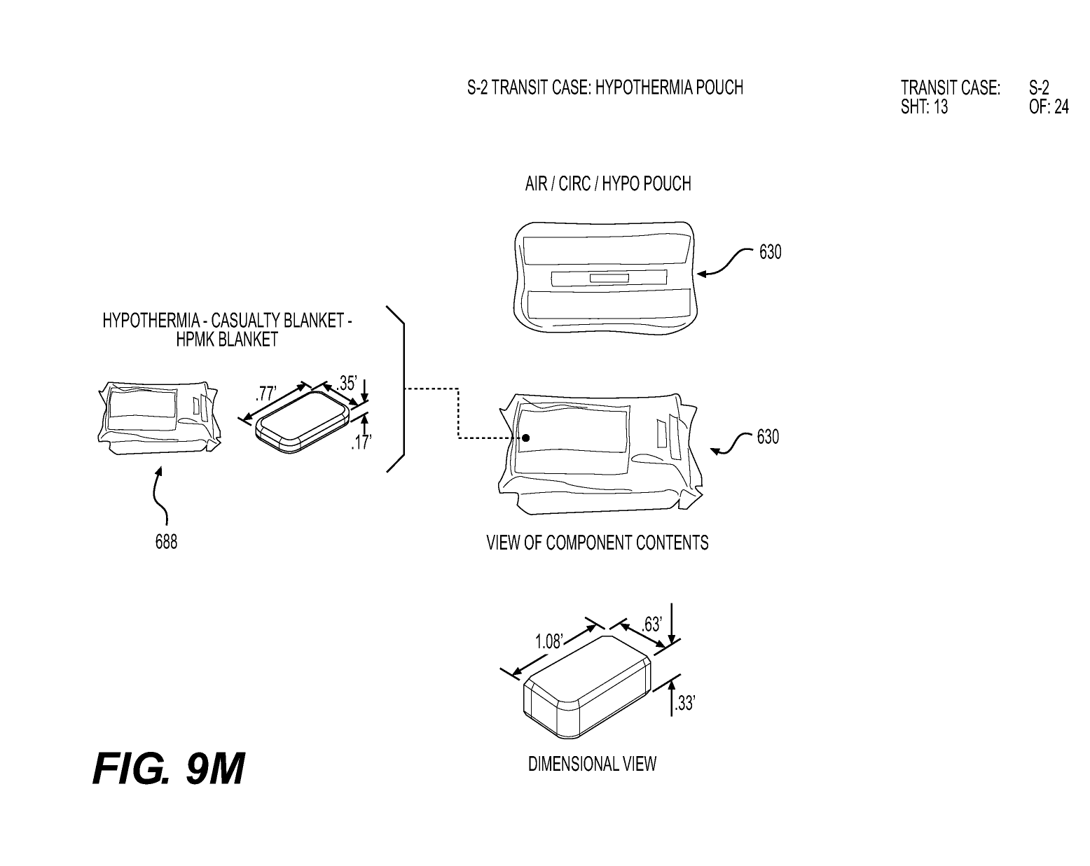

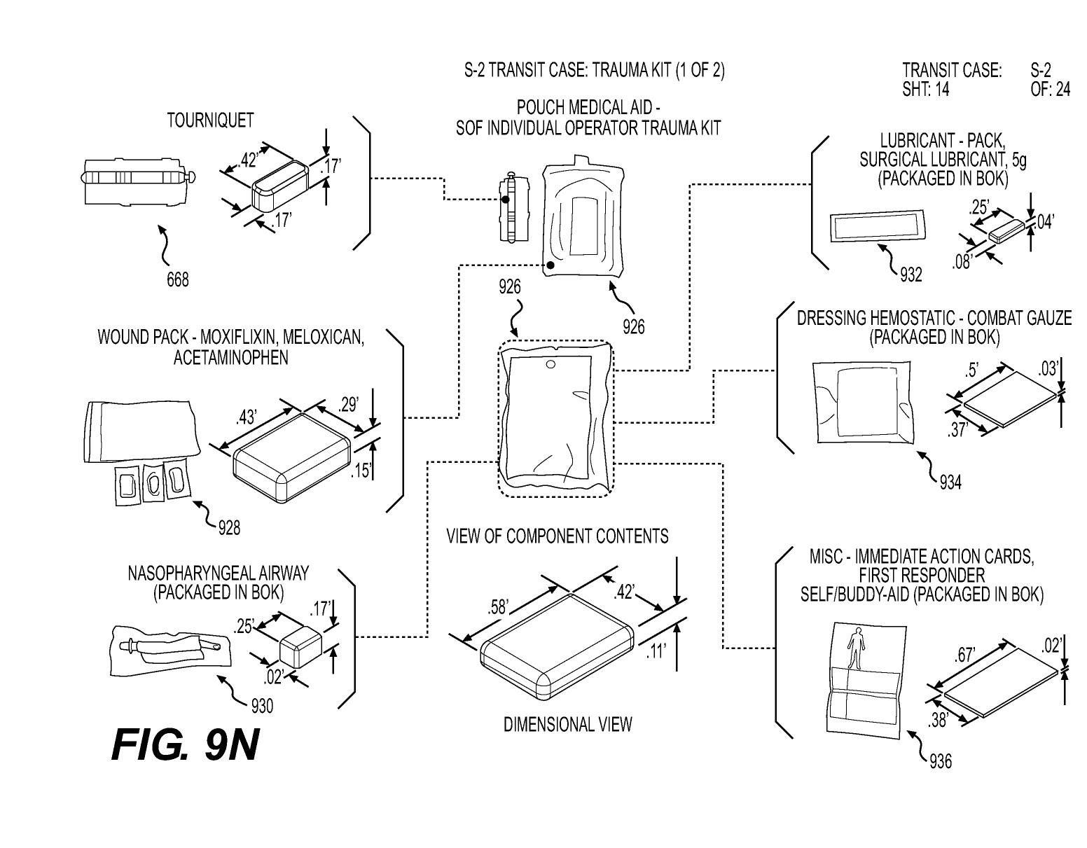

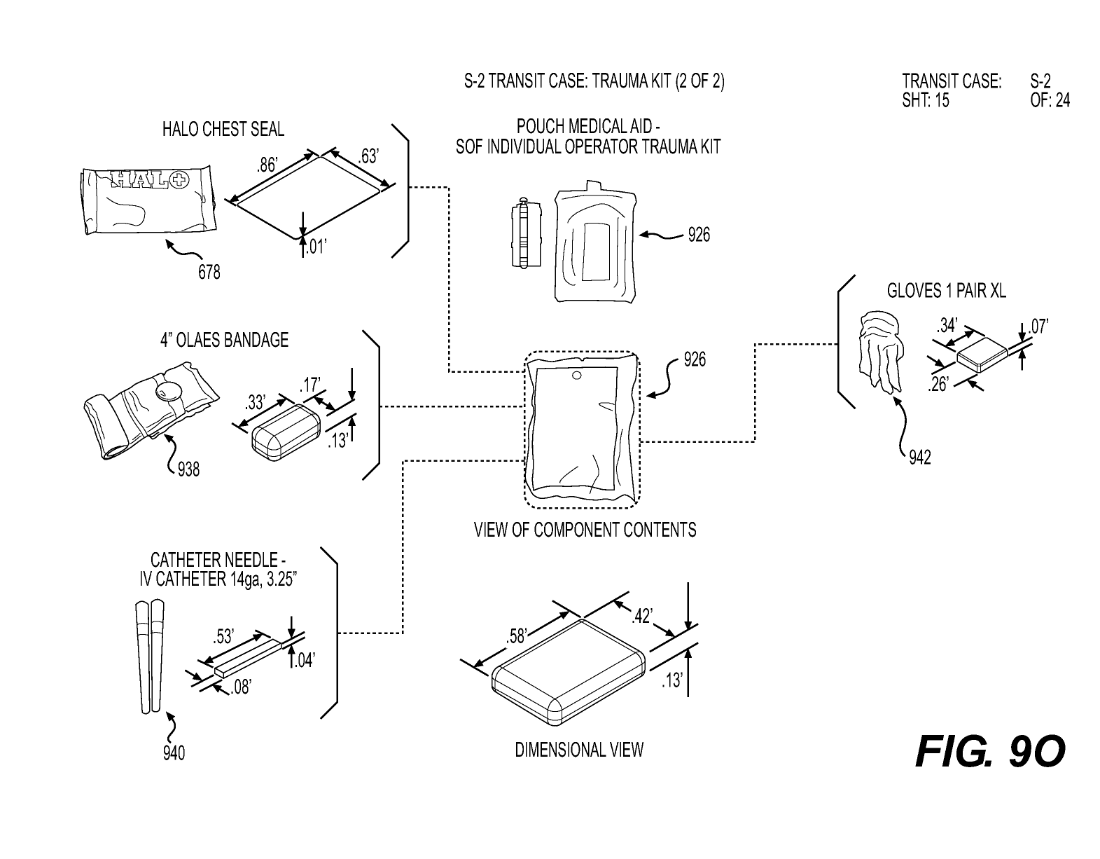

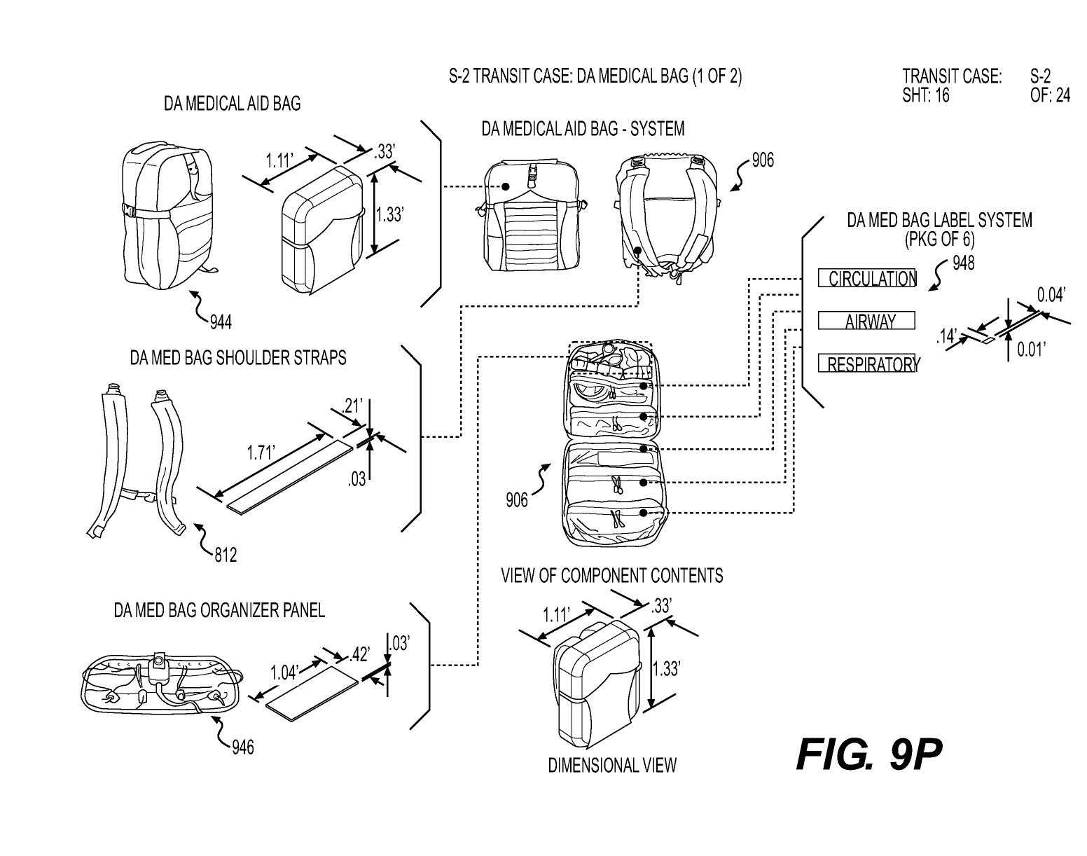

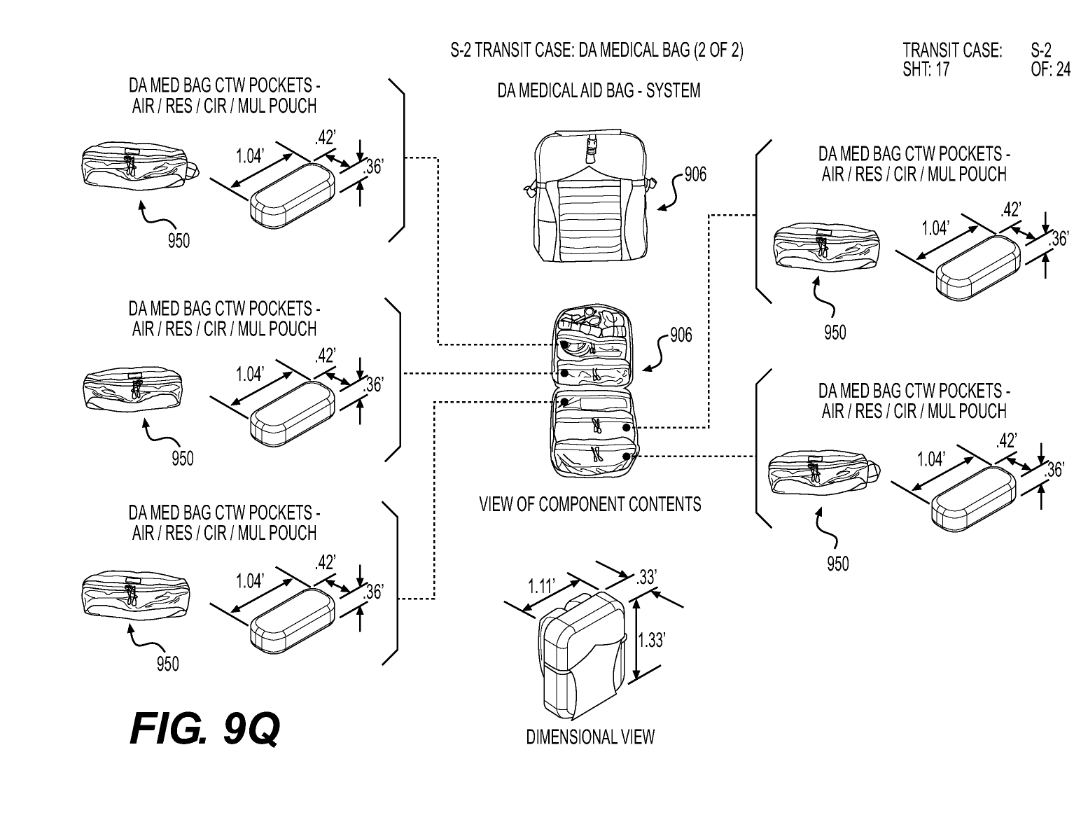

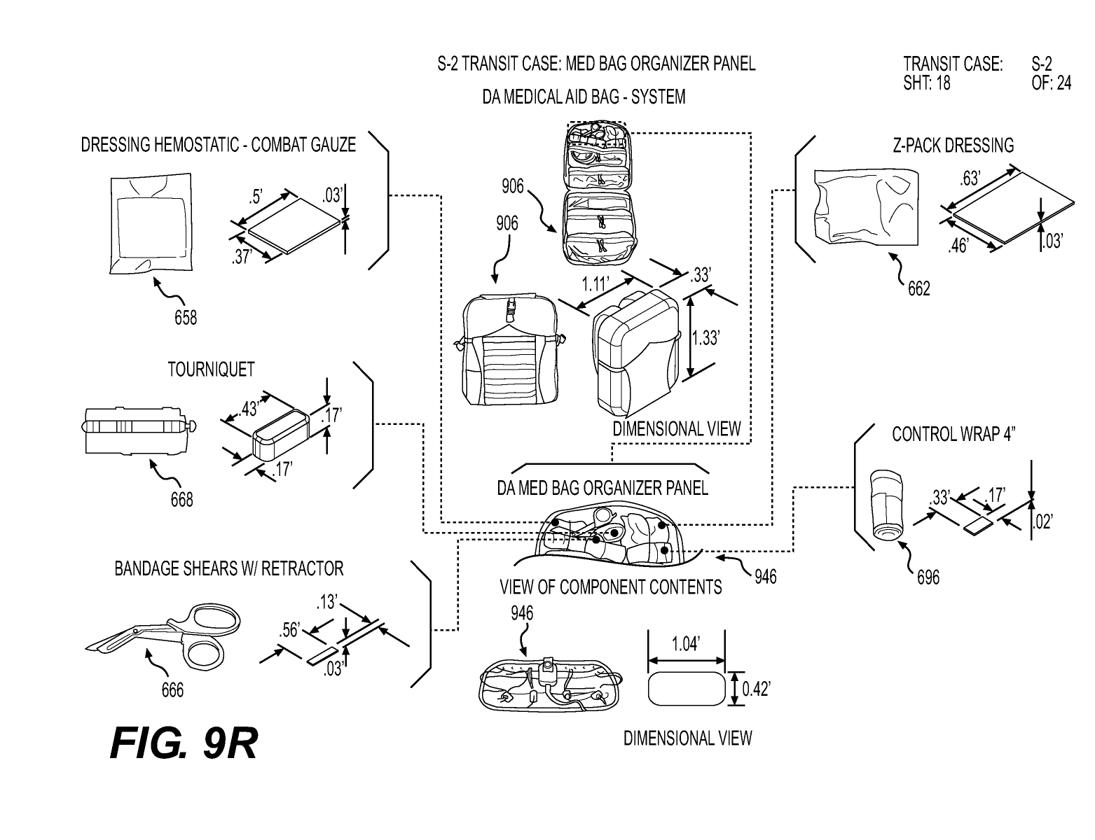

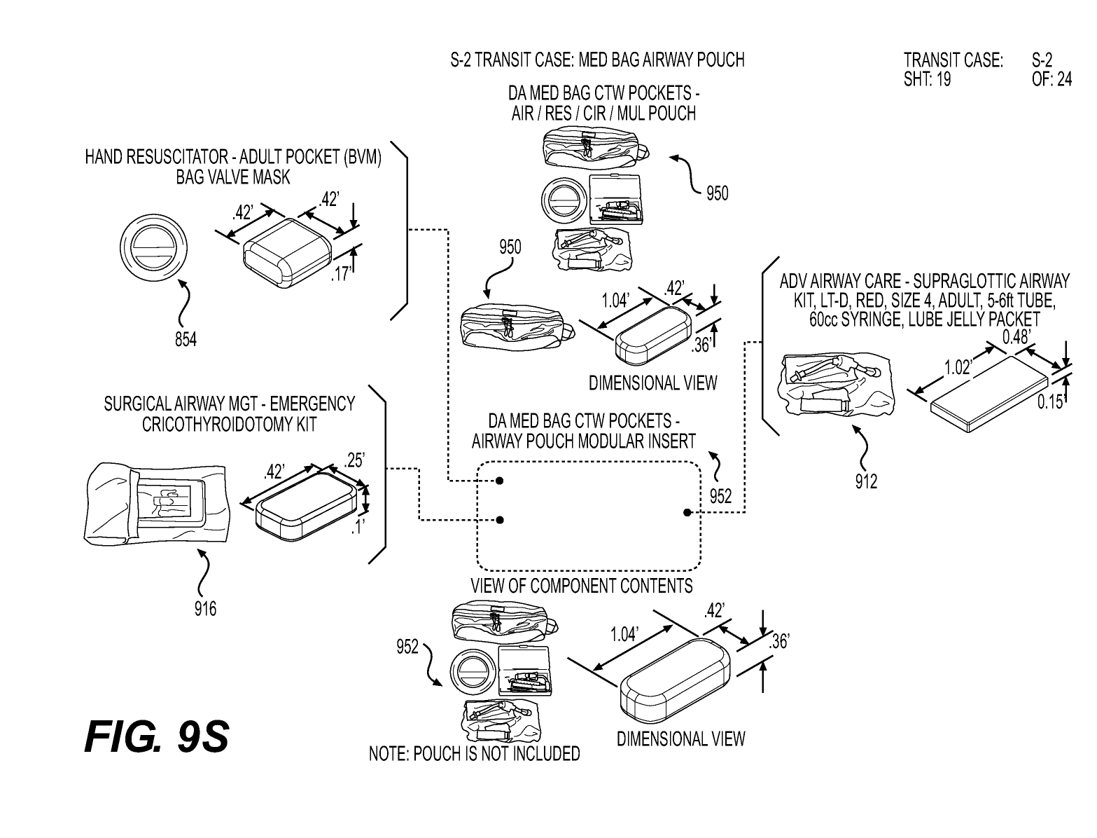

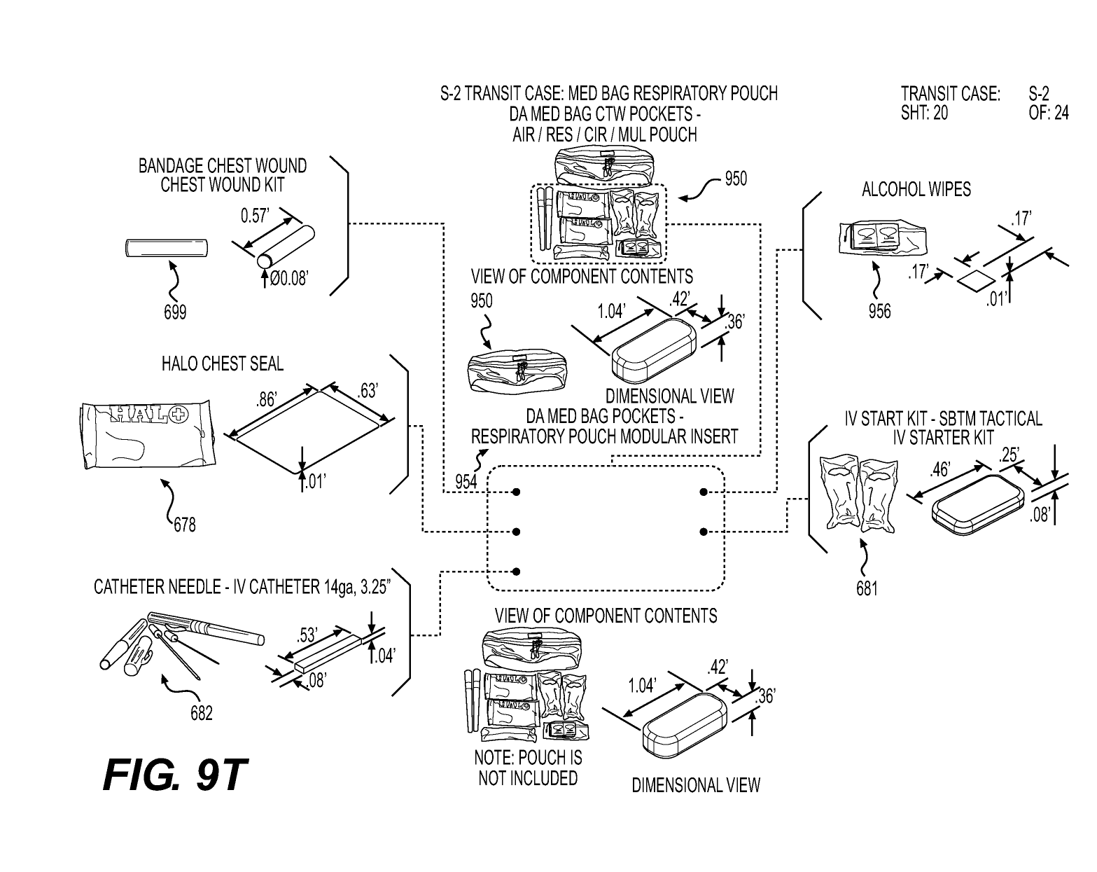

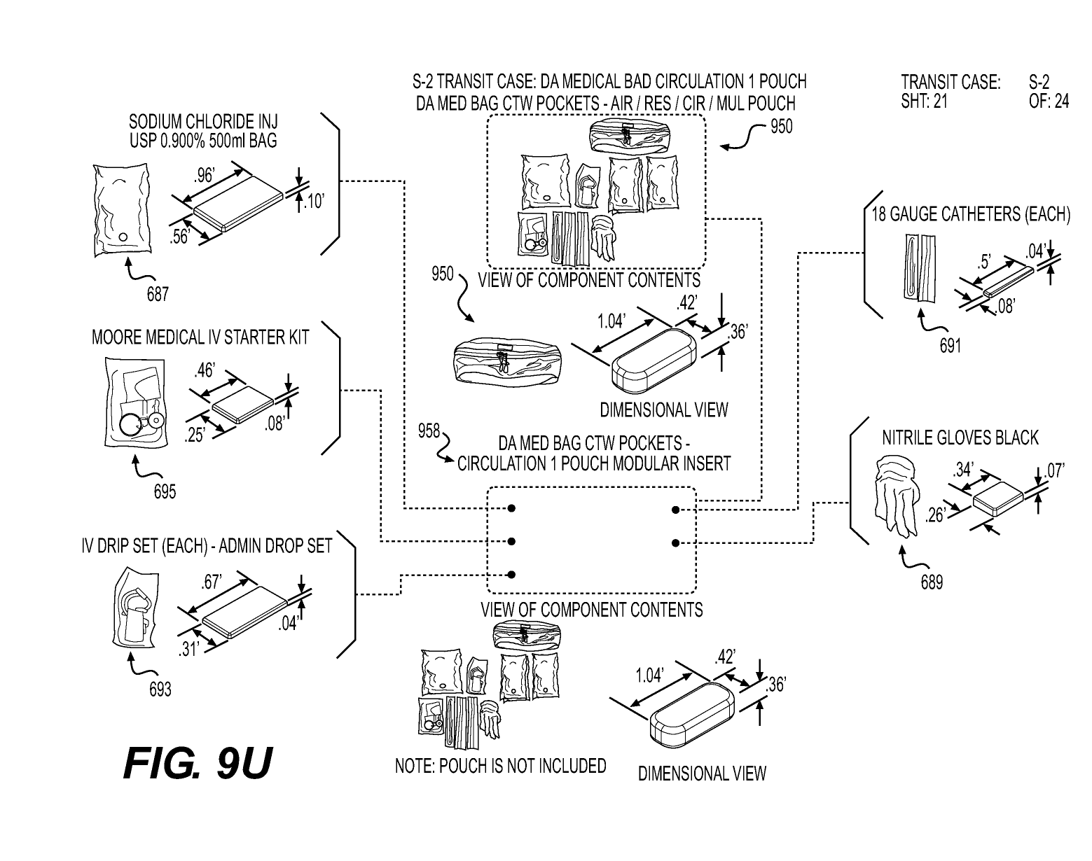

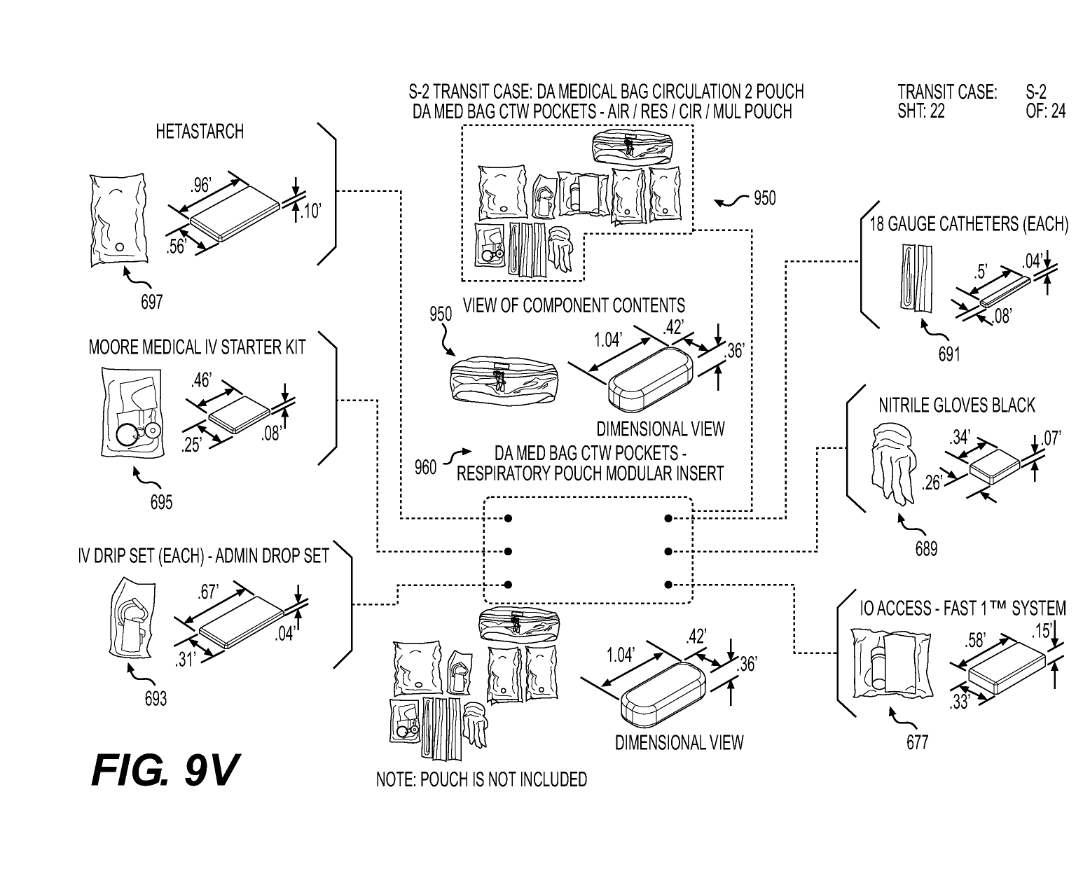

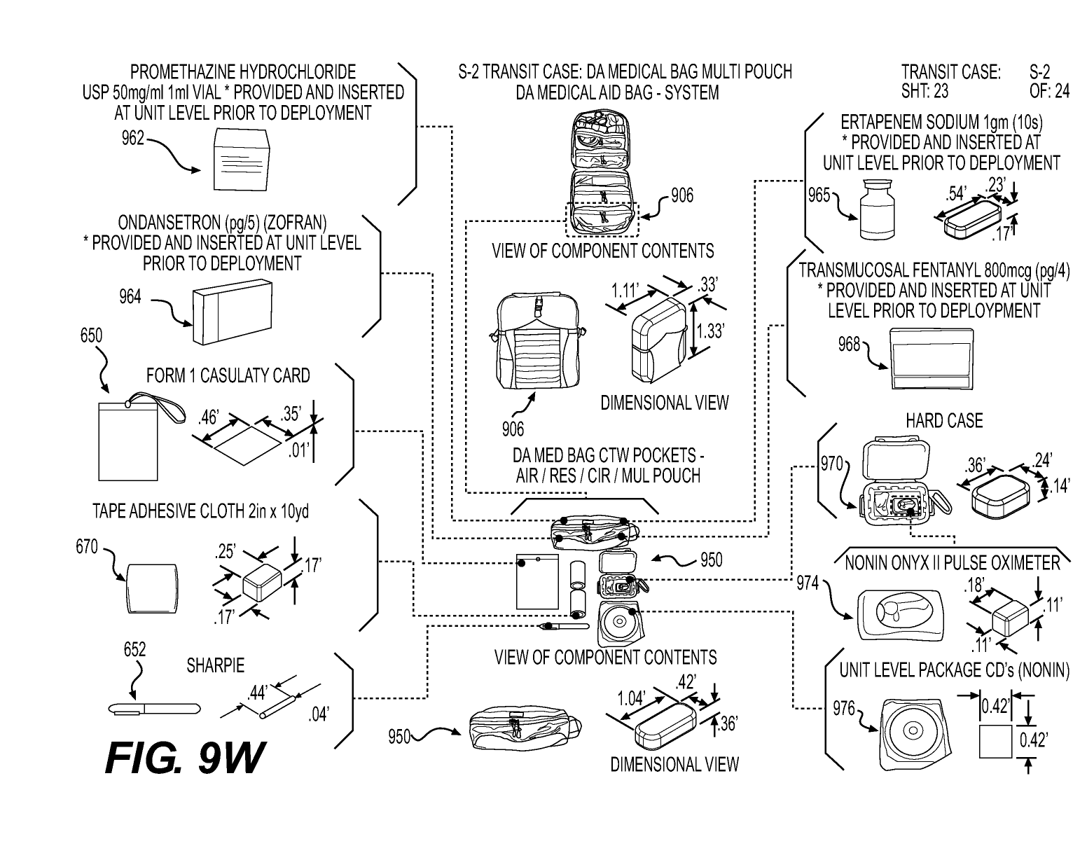

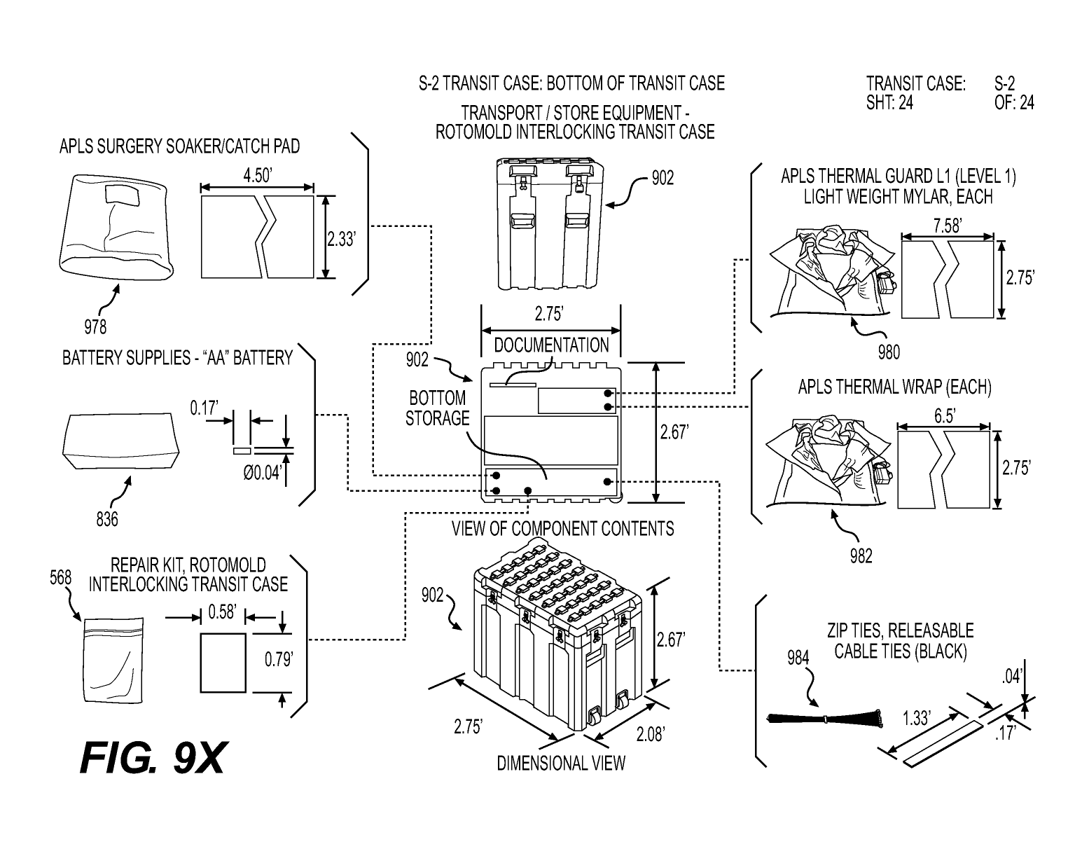

FIG. 9A illustrates exemplary case contents of the S-2 transit case 148 of the sustainment kit. Transport/Store Equipment—Rotomold Interlocking Transit Case 902 is shown containing Man-Packable Mounted/Dismounted—Pack System 904 and DA Medical Aid Bag—System 906. Transport/Store Equipment—Rotomold Interlocking Transit Case 902 may be NSN 8145-01-606-2292. Man-Packable Mounted/Dismounted—Pack System 904 may be NSN 8465-01-607-0566, 0568, 0571 or 0575. DA Medical Aid Bag—System 906 may be NSN 8465-01-607-0577, 0586, 0593 or 0598.

FIG. 9B illustrates the exemplary Pack System of the S-2 transit case 148. This is the first of two figures showing this pack system, the second being FIG. 9C below. Man-Packable Mounted/Dismounted—Pack System 904 is shown containing DA Med Bag Shoulder Straps 812, Pack System—Base 414, Pouch MPD 5-10-2.5 626, Standard Flap—Reversible 482 and Flatbed Full Panel Sustainment Kit Panel 814. This sustainment panel may be two panels side-by-side as shown on the Figure, similar to as described above.

FIG. 9C illustrates the exemplary Pack System of the S-2 transit case 148. This is the second of two figures showing this pack system, the first being FIG. 9B above. Man-Packable Mounted/Dismounted—Pack System 904 is shown containing IFAK/Strap/Mass Hem/Resp Pouch 628 (×2), Sustainment—WWTK Pouch 816, Air/Circ/Hypo Pouch 630 (×3) and Label System—S-2 (PKG of 8) 908. Label System—S-2 (PKG of 8) 908 may be NSN 7690-01-607-0563. The dual sustainment panels are shown on this figure too.

FIG. 9D illustrates the exemplary Lower Pouch of the S-2 transit case 148. Pouch MPD 5-10-2.5 626 is shown containing Headlamp Remix Pro, Red/Green/IR led/White (Tan Headstrap) 648, Emergency Lighting—LazerBrite Multi-Lux (low, high, flash)—Red & Infrared 642, Emergency Lighting—LazerBrite Multi-Lux (low, high, flash)—Blue & Green 644, Forceps curved—Kelly Forcep, 5½″ Curved 830, Form 1 Casualty Card 650 and Sharpie 652.

FIG. 9E illustrates the exemplary IFAK Pouch of the S-2 transit case 148. This is the first of two figures showing this pouch, the second being FIG. 9F below. IFAK/Strap/Mass Hem/Resp Pouch 628 is shown containing Sustainment 2 IFAK Pouch Modular Insert 656. Sustainment 2 IFAK Pouch Modular Insert 656 is shown containing Dressing Hemostatic—Combat Gauze 658, Petroleum Glaze 660, Z-Pack Dressing 662, Vionex Hand Wipes Each 664, Bandage Shears with Retractor 666, Tourniquet 668, Control Wrap 4″ 696, Tape Adhesive Cloth 2 in×10 yd 670, Nasopharyngeal Airway 672 and Lubricant—Pack, Surgical Lubricant, 5g 674.

FIG. 9F illustrates the exemplary IFAK Pouch of S-2 transit case 148. This is the second of two figures showing this pouch, the first being FIG. 9E above. IFAK/Strap/Mass Hem/Resp Pouch 628 is shown containing Sustainment 2 IFAK Pouch Modular Insert 656. Sustainment 2 IFAK Pouch Modular Insert 656 is shown containing Gloves—Blood ID—Nitrile Gloves (OD), XL, In heavy Mil zip-lock bag 676, Halo Chest Seal 678, Chest Dressing—6″ Chest Dressing (Isreali) 680 and Catheter Needle—IV Catheter 14GA, 3.25″ 682.

FIG. 9G illustrates the exemplary Airway Pouch of the S-2 transit case 148. This is the first of two figures showing this pouch, the second being FIG. 6H below. Air/Circ/Hypo Pouch 630 is shown containing Sustainment 2—Airway Pouch Modular Insert 910. Sustainment 2—Airway Pouch Modular Insert 910 is shown containing Handle Fiber Optic—Handle, Laryngoscope Fiber Optic, “AA” Size lightweight 834, Battery Supplies—“AA” Battery 836, Handle Fiber Optic—Bulb, “AA” Size Lightweight Fiber Optic Laryngoscope Handle 842, Forceps Angled—Magill Forceps 844, Laryngoscope Blade Metal Durable—Blade, Laryngoscope FO Miller No. 3 838, Laryngoscope Blade Metal Durable—Blade, Laryngoscope FO English No. 3 840, Adv Airway Care—Supraglottic Airway Kit, LT-D, red, size 4, adult, 5-6 ft tube, 60 cc syringe, lube jelly packet 912 and Advanced Airway—7.5 MM Endotracheal Tube insertion package, complete with stylet, 10 cc syringe and tube securing device 848. Battery Supplies—“AA” Battery 836 may be NSN 3120008370887. Sustainment 2—Airway Pouch Modular Insert 910 may be NSN 6545-01-607-9756. Adv Airway Care—Supraglottic Airway Kit, LT-D, red, size 4, adult, 5-6 ft tube, 60 cc syringe, lube jelly packet 912 may be NSN 6515-01-515-0151. The note on the figure that the pouch is not included referes to the modular insert so that the recipient or user can replenish the contents without ordering a new nylon pouch.

FIG. 9H illustrates the exemplary Airway Pouch of the S-2 Transit Case 148. This is the second of two Figures showing this, the first being FIG. 9G. Air/Circ/Hypo Pouch 630 is shown containing Sustainment 2—Airway Pouch Modular Insert 910. Sustainment 2—Airway Pouch Modular Insert 910 is shown containing Advanced Airway—8.0 MM Endotracheal tube insertion package, complete with stylet, 10 CC Syringe and Et tube securing device 850, Suction Kit Manual Disposable—Suction easy with suction catheter adapter 852, Adv Airway Care—Kit, Boundtree Nasal Airway 858, Hand Resuscitator—Adult Pocket (BVM) Bag Valve Mask 854, Adv Airway Care—Kit, Boundtree Berman Airway kit 914 and Surgical Airway Mgt—Emergency Cricothyroidotomy Kit 916. Adv Airway Care—Kit, Boundtree Berman Airway kit 914 may be NSN 6515-01-518-8593. Surgical Airway Mgt—Emergency Cricothyroidotomy Kit 916 may be NSN 6515-01-607-9809.

FIG. 9I illustrates the exemplary Respiratory Pouch of the S-2 transit case 148. IFAK/Strap/Mass Hem/Resp Pouch 628 is shown containing Sustainment 2—Respiratory Pouch Modular Insert 918. Sustainment 2—Respiratory Pouch Modular Insert 918 is shown containing Bandage Chest Wound Chest Wound Kit 699, Halo Chest Seal 678, Gloves—Blood ID—Nitrile Gloves (OD), XL, In heavy Mil Zip lock bag 676, Z-Pack Dressing 662, Dressing Abdominal, Emergency Abdominal 692, Chest Dressing—6″ Chest Dressing (Israeli) 680 and Adv Airway Care—H&H Chest Tube Insertion Kit 920. Sustainment 2—Respiratory Pouch Modular Insert 918 may be NSN 6545-01-607-9344. Adv Airway Care—H&H Chest Tube Insertion Kit 920 may be NSN 6515-01-579-3275.

FIG. 9J illustrates the exemplary Circulation Pouch of the S-2 transit case 148. This is the first of two figures showing this pouch, the second being FIG. 6L below. Air/Circ/Hypo Pouch 630 is shown containing Circulation Pouch Modular Insert 673. Circulation Pouch Modular Insert 673 is shown containing Hetastarch 697, Moore Medical IV Starter Kit 695 (×2), IV Drip Set (each) Admin Drop set 693 (×2), 18 Gauge Catheters 691 (×2), Nitrile Gloves Black 689 (×2) and Sodium Chloride Inj USP 0.900% 50 ml Bag 687.

FIG. 9K illustrates the exemplary Circulation Pouch of the S-2 transit case 148. This is the second of two figures showing this pouch, the first being FIG. 9J above. Air/Circ/Hypo Pouch 630 is shown containing Sustainment 2—Circulation Pouch Modular Insert 922. Sustainment 2—Circulation Pouch Modular Insert 922 is shown containing Ringer's Inj Lactated USP 500 ml Bag 685, Moore Medical IV Starter Kit 695, IV Drip Set (each)—Admin Drop Set 693, 18 Gauge Catheters 691, Nitrile Gloves Black 689, Protective Set Combat IV Coban 683, IV Start Kit—SBTM Tactival IV Starter Kit 681, Syringe—Syringe, 10 CC Luer Lock 679 and IO Access—Fast 1 (trademark) system 677. Sustainment 2—Circulation Pouch Modular Insert 922 may be NSN 6545-01-607-9335.

FIG. 9L illustrates the exemplary WWTK Pouch of the S-2 transit case 148. Sustainment—WWTK Pouch 816 is shown containing Sustainment 2—WWTK Pouch Modular Insert 924. Sustainment 2—WWTK Pouch Modular Insert 924 is shown containing Syringe—Syringe 10 CC Luer Lock 679, Assortment of Injection Needles (18GA) 866, Assortment of Injection Needles (21GA) 868, Burn Bandage—Dressing, Antimicrobial Burn Wrap, 4″×66″ 694 and Sage P2 Sharps Shuttle 870. Sustainment 2—WWTK Pouch Modular Insert 924 may be NSN 6545-01-607-9338.

FIG. 9M illustrates the exemplary Hypothermia Pouch of the S-2 transit case 148. Air/Circ/Hypo Pouch 630 is shown containing Hypothermia—Casualty Blanket—HPMK Blanket 688.

FIG. 9N illustrates the exemplary Trauma Kit of the S-2 transit case 148. This is the first of two figures showing this trauma kit, the second being FIG. 9O below. Pouch Medical Aid—SOF Individual Operator Trauma Kit 926 is shown containing Tourniquet 668, Would Pack—Moxiflixin, Meloxican, Acetaminophen 928, Nasopharyngeal Airway (Packaged in BOK) 930, Lubricant—Pack, Surgical Lubricant, 5g (Packaged in BOK (Blow out kit) 932, Dressing Hemostatic—Combat Gauze (Packaged in BOK) 934 and Immediate Action Cards, First responder self/buddy-aid (Packaged in BOK) 936.

FIG. 9O illustrates the exemplary Trauma Kit of the S-2 transit case 148. This is the second of two figures showing this trauma kit, the first being FIG. 9N above. Pouch Medical Aid—SOF Individual Operator Trauma Kit 926 is shown containing Halo Chest Seal 678, 4″ Olaes Bandage 938, Catheter Needle—IV Catheter 14GA, 3.25″ 940 and Gloves 1 pair xl 942. Catheter Needle—IV Catheter 14GA, 3.25″ 940 may be NSN 6515-01-0601-1292.

FIG. 9P illustrates the exemplary DA Medical Bag of the S-2 transit case 148. This is the first of two figures showing this bag, the second being FIG. 9Q below. DA Medical Aid Bag—System 906 is shown containing DA Medical Bag 944, DA Med Bag Shoulder Straps 812, DA Med Bag Organizer Panel 946 and DA Med Bag Label System (PKG of 6) 948. DA Medical Bag 944 may be NSN 8465-01-607-0585, 0591, 0597 or 0601. DA Med Bag Organizer Panel 946 may be NSN 8465-01-607-0631, 0635, 0649 or 0674. DA Med Bag Label System (PKG of 6) 948 may be NSN 7690-01-607-0768.

FIG. 9Q illustrates the exemplary DA Medical Bag of the S-2 transit case 148. This is the second of two figures showing this bag, the first being FIG. 6P above. DA Medical Aid Bag—System 906 is shown containing DA Med Bag Pockets—Air/Res/Cir/Mul Pouch 950 (×5). DA Med Bag Pockets—Air/Res/Cir/Mul Pouch 950 may be NSN 8465-01-607-0757, 0758, 0777 or 0763.

FIG. 9R illustrates the exemplary Med Bag Organizer Panel of the S-2 transit case 148. DA Medical Aid Bag—System 906 is shown containing DA Med Bag Organizer Panel 946. DA Med Bag Organizer Panel 946 is shown containing Dressing Hemostatic—Combat Gauze 658, Tourniquet 668, Bandage Shears with Retractor 666, Z-Pack Dressing 662 and Control Wrap 4″ 696.

FIG. 9S illustrates the exemplary Med Bag Airway Pouch of the S-2 transit case 148. DA Med Bag Pockets—Air/Res/Cir/Mul Pouch 950 is shown containing DA Med Bag Pockets—Airway Pouch Modular Insert 952. DA Med Bag Pockets—Airway Pouch Modular Insert 952 is shown containing Hand Resuscitator—Adult Pocket (BVM) Bag Valve Mask 854, Surgical Airway Mgt—Emergency Cricothyroidotomy Kit 916 and Adv Airway Care—Supraglottic Airway Kit, LT-D, red, size 4, adult, 5-6 ft tube, 60 cc syringe, lube jelly packet 912. DA Med Bag Pockets—Airway Pouch Modular Insert 952 may be NSN 6545-01-607-9324.

FIG. 9T illustrates the exemplary Med Bag Respiratory Pouch of the S-2 transit case 148. DA Med Bag Pockets—Air/Res/Cir/Mul Pouch 950 is shown containing DA Med Bag Pockets—Respiratory Pouch Modular Insert 954. DA Med Bag Pockets—Respiratory Pouch Modular Insert 954 is shown containing Bandage Chest wound Chest wound kit 699, Halo Chest Seal 678, Catheter Needle—IV Catheter 14GA, 3.25″ 682, Alcohol Wipes 956 and IV Starter Kit—SBTM Tactical IV Starter Kit 681. DA Med Bag Pockets—Respiratory Pouch Modular Insert 954 may be NSN 6545-01-607-9306. Alcohol Wipes 956 may be NSN 6510-01-497-0978.

FIG. 9U illustrates the exemplary DA Medical Bag Circulation 1 Pouch S-2 transit case 148. DA Med Bag Pockets—Air/Res/Cir/Mul Pouch 950 is shown containing DA Med Bag Pockets—Circulation 1—Pouch Modular Insert 958. DA Med Bag Pockets—Circulation 1—Pouch Modular Insert 958 is shown containing Sodium Chloride Inj USP 0.900% 500 ml Bag 687, Moore medical IV Starter Kit 695, IV Drip Set (each)—Admin Drop set 693, 18 Gauge Catheters (each) 691 and Nitrile Gloves Black 589. DA Med Bag Pockets—Circulation 1—Pouch Modular Insert 958 may be NSN 6545-01-608-2550.

FIG. 9V illustrates the exemplary DA Medical Bag Circulation 2 Pouch of the S-2 transit case 148. DA Med Bag Pockets—Air/Res/Cir/Mul Pouch 950 is shown containing DA Med Bag Pockets—Circulation 2—Pouch Modular Insert 960. DA Med Bag Pockets—Circulation 2—Pouch Modular Insert 960 is shown containing Hetastarch 697, Moore Medical IV Starter Kit 695, IV Drip Set (each)—Admin Drop Set 693, 18 Gauge Catheters (each) 691, Nitrile Gloves Black 689, and IO Access—Fast 1 (trademark) system 677. DA Med Bag Pockets—Circulation 2—Pouch Modular Insert 960 may be NSN 6545-01-608-2696.

FIG. 9W illustrates the exemplary DA Medical Bag Multi Pouch of the S-2 transit case 148. DA Medical Aid Bag—System 906 is shown containing DA Med Bag Pockets—Air/Res/Cir/Mul Pouch 950. DA Med Bag Pockets—Air/Res/Cir/Mul Pouch 950 is shown containing Promethazine Hydrochloride USP 50 mg/ml 1 ml Vial 962, Ondanestron (pg/5) (Zofran) 964, Form 1 Casualty Card 650, Tape Adhesive Cloth 2 in×10 yd 670, Sharpie 652, Ertapenem Sodium 1GM (10 s) 965, Transmucosal Fentanyl 800 mcg (pg/4) 968, Hard Case 970, Nonin Onyx II Pulse Oximeter 974 and Unit Level Package CD's (Nonin) 976. Promethazine Hydrochloride USP 50 mg/ml 1 ml Vial 962 may be NSN NDC 641149635. Hard Case 972 may be NSN 6515-01-519-7862. Nonin Onyx II Pulse Oximeter 974 may be NSN 6515-01-535-2728. Unit Level Package CD's (Nonin) 976 may be NSN 6515-01-535-2728.

FIG. 9X illustrates the exemplary bottom portion of the S-2 transit case 148. Transport/Store equipment—Rotomold Interlocking Transit Case 902 is shown containing APLS Surgery Soaker/Catch Pad 978, Battery Supplies—“AA” Battery 836, Repair Kit, Rotomold Interlocking Transit Case 568, APLS Thermal Guard L1 (Level 1) Light Weight Mylar, Each 980, APLS Thermal Wrap (each) 982 and Zip ties, releasable Cable Ties (Black) 984. LS Surgery Soaker/Catch Pad 978 may be NSN 6530-01-561-7741. APLS Thermal Wrap (each) 982 may be NSN 6530-01-576-6389. Zip ties, releasable Cable Ties (Black) 984 may be NSN 5975-01-608-3692.

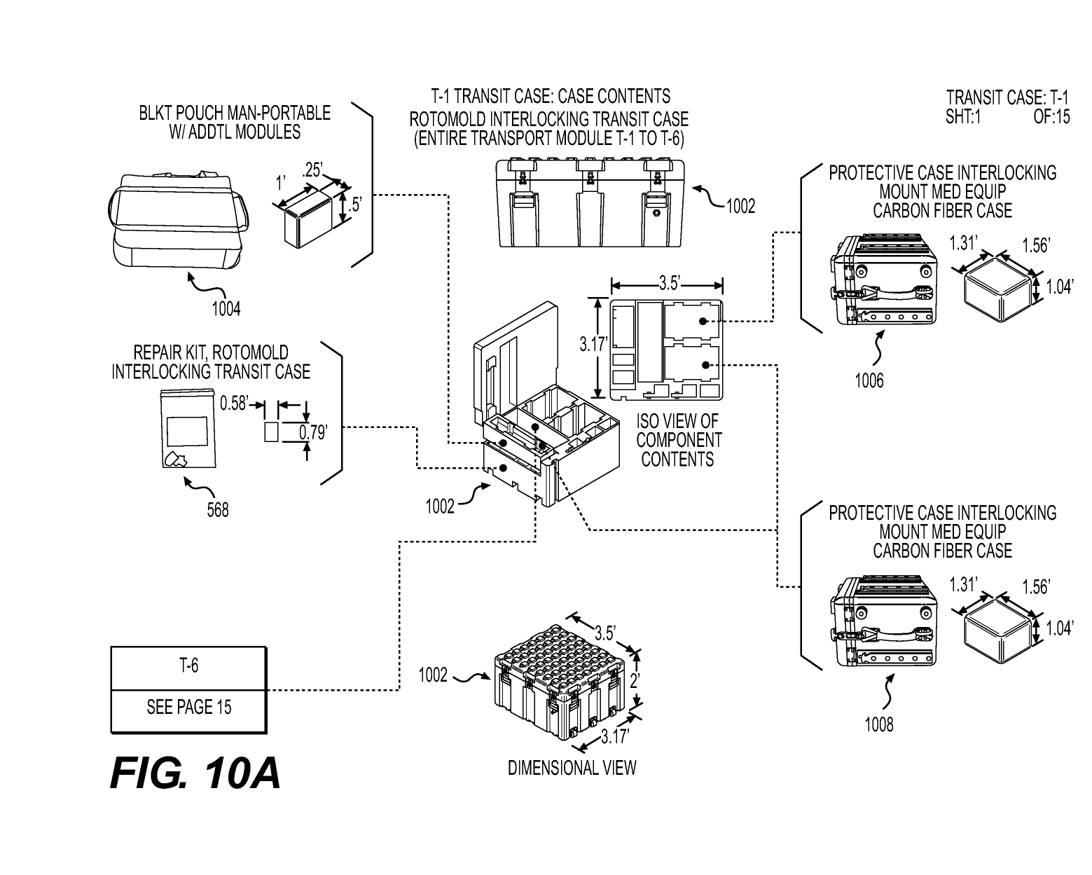

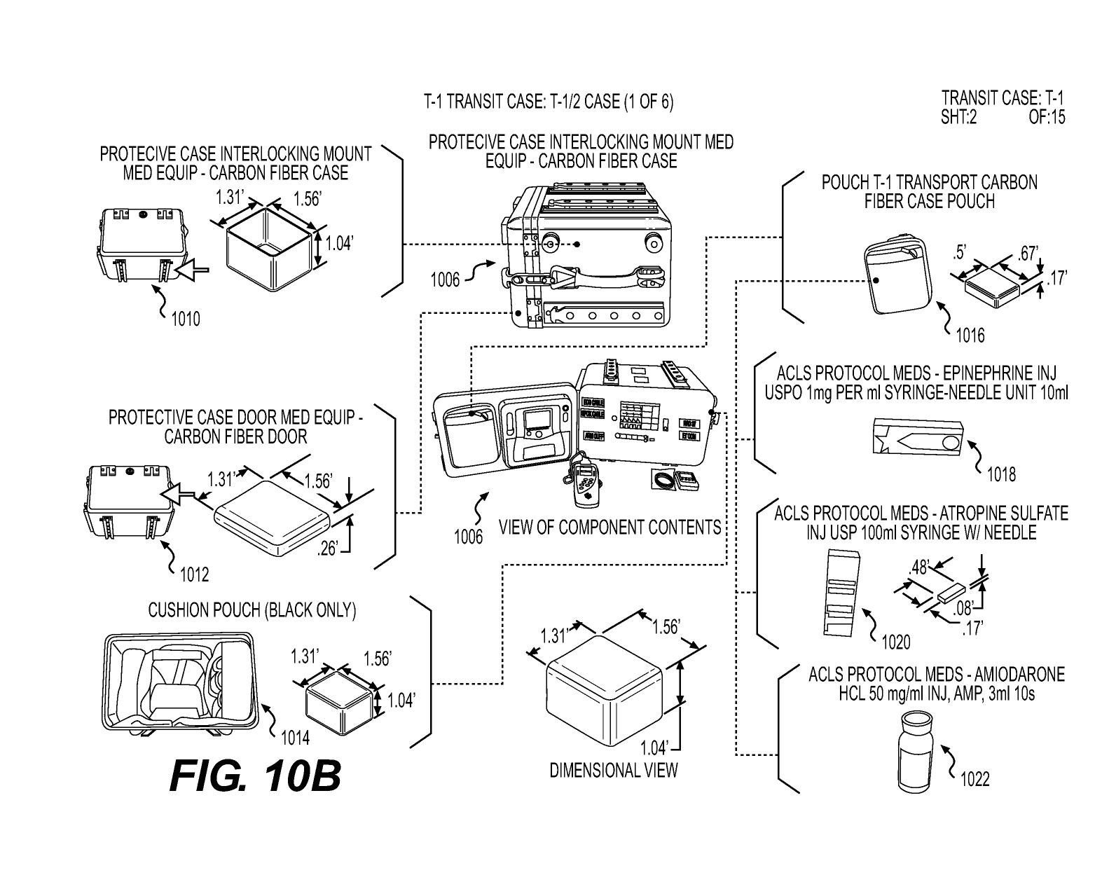

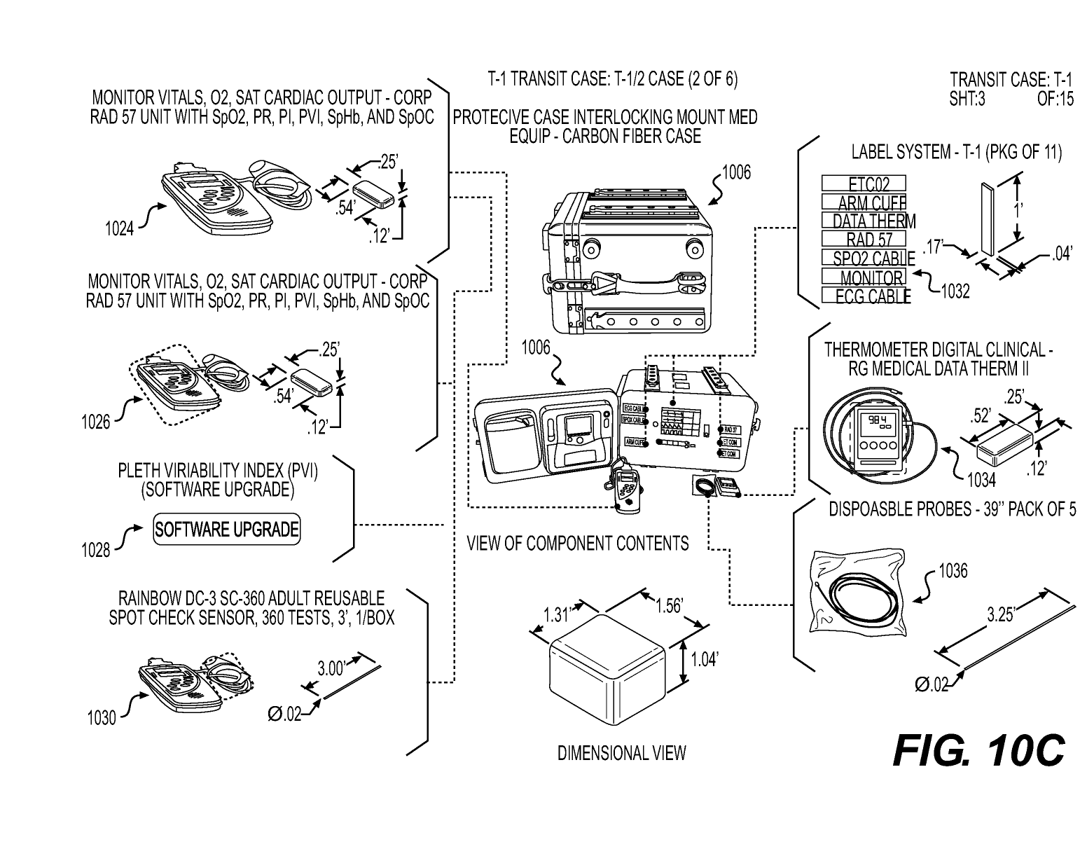

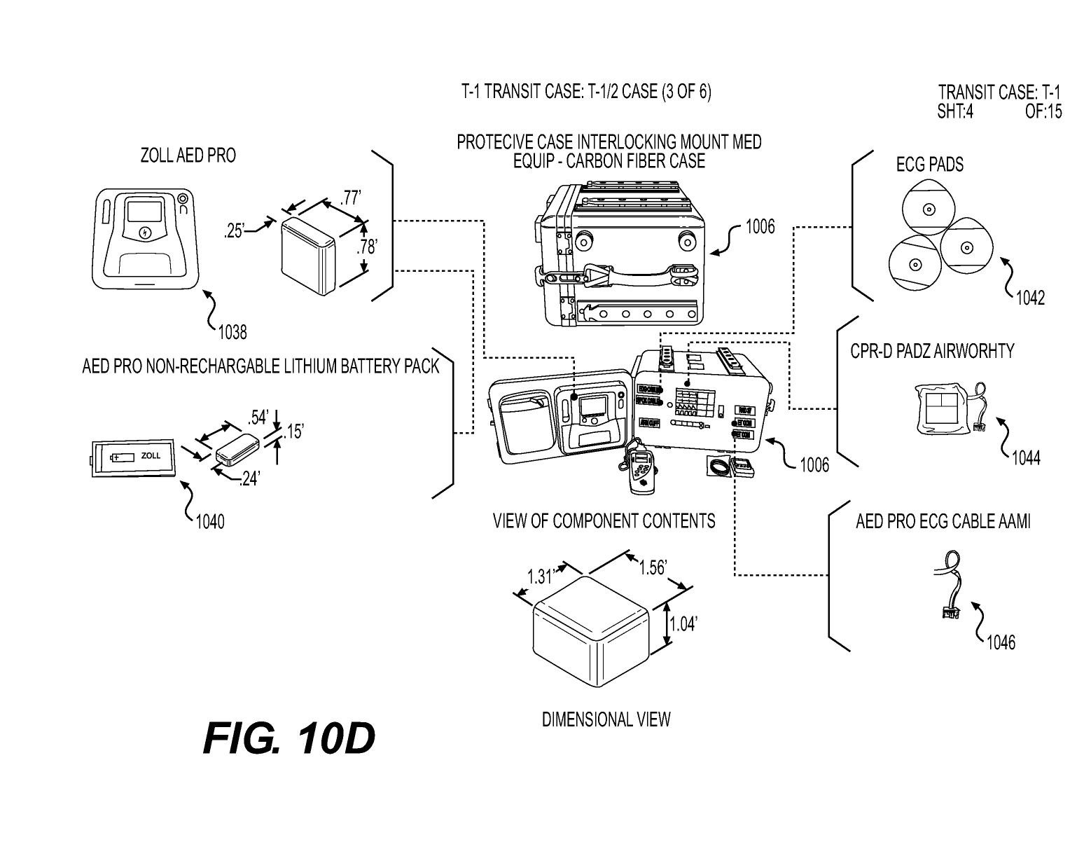

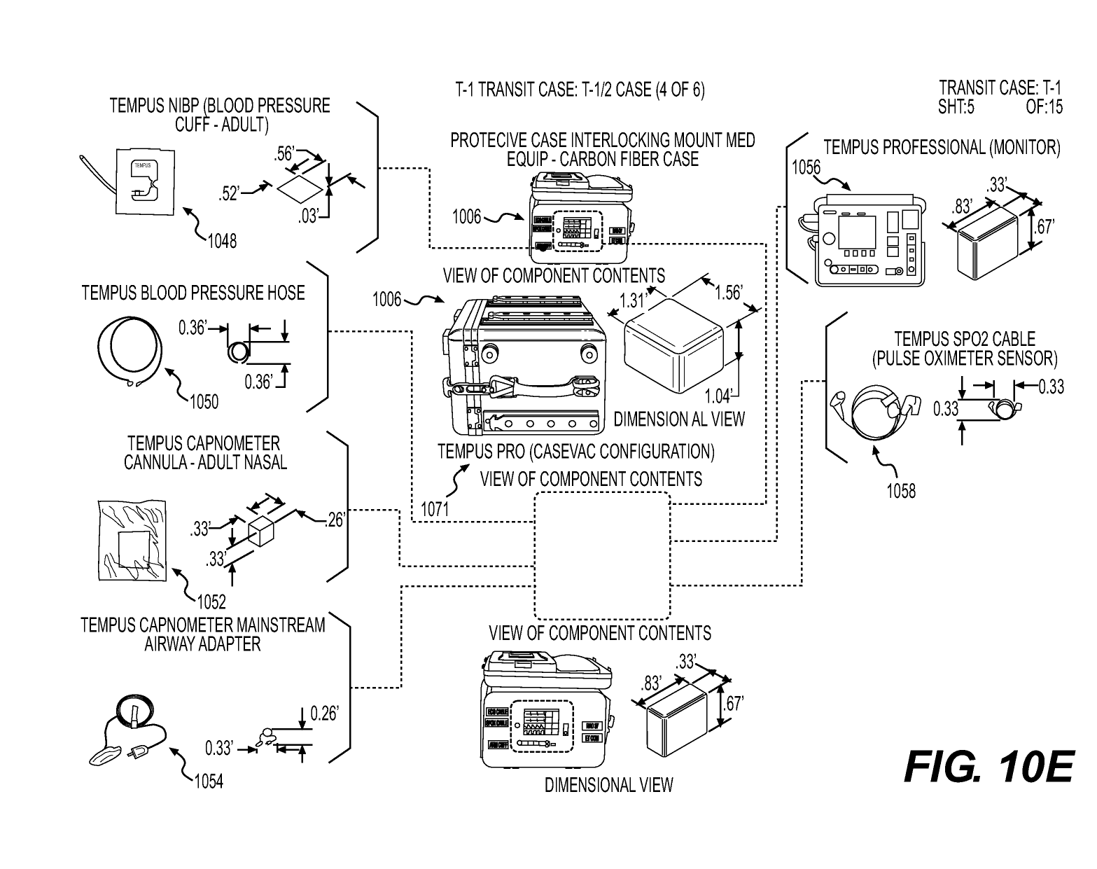

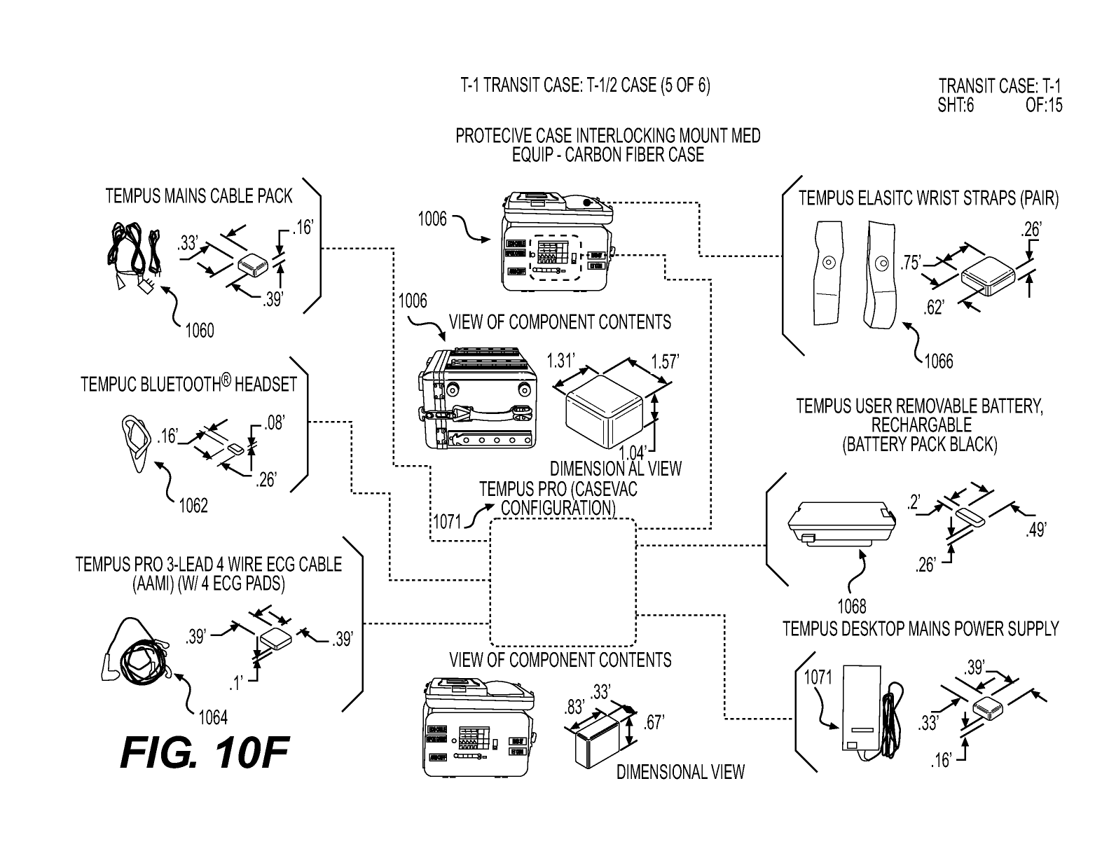

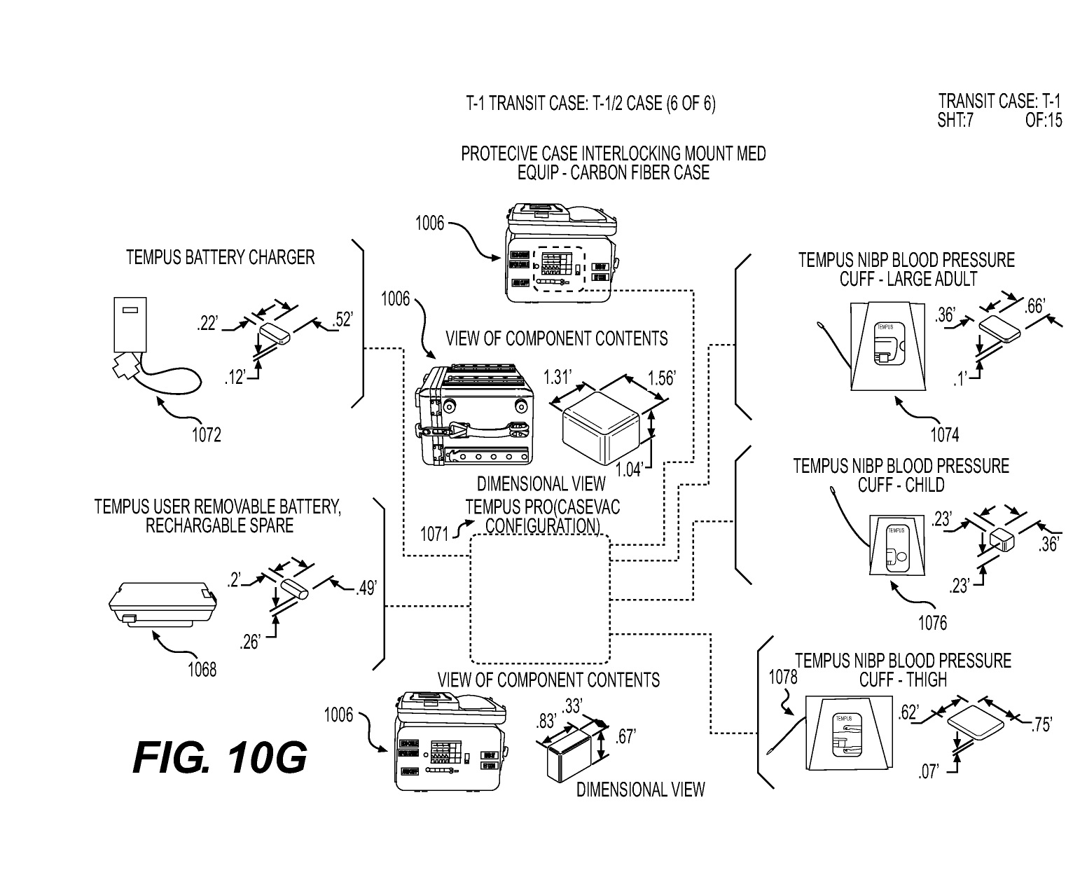

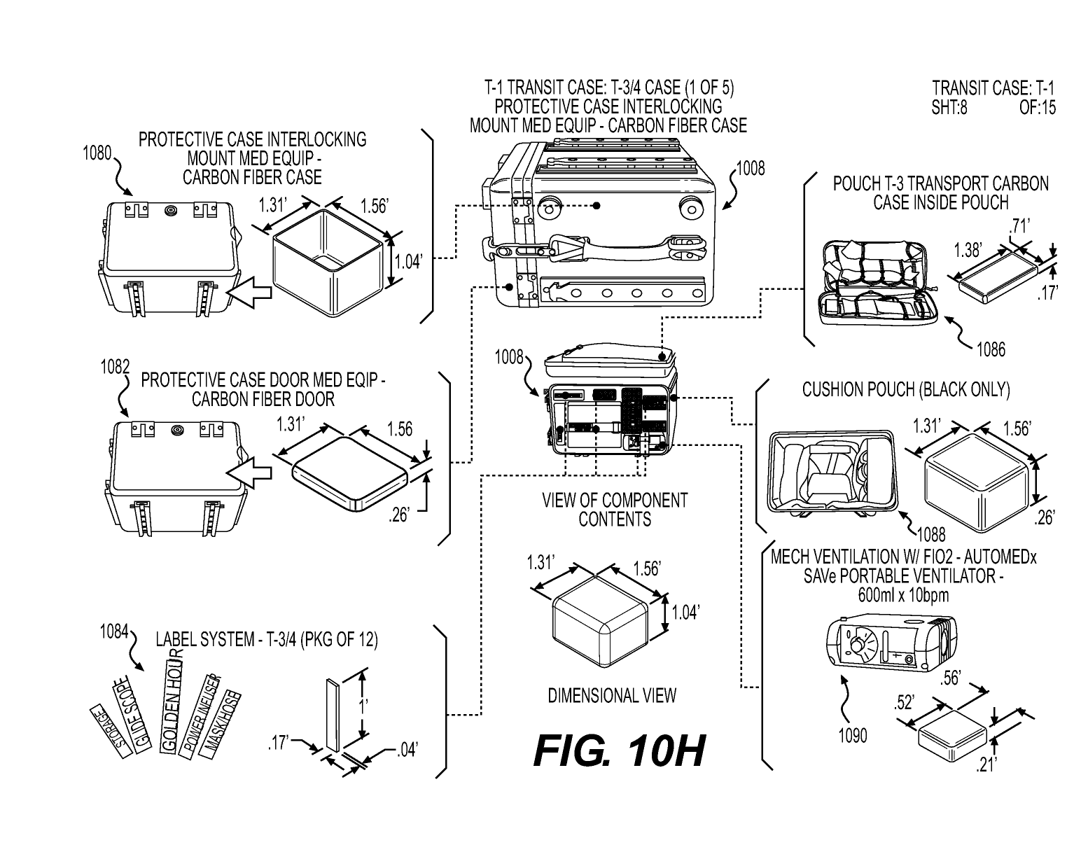

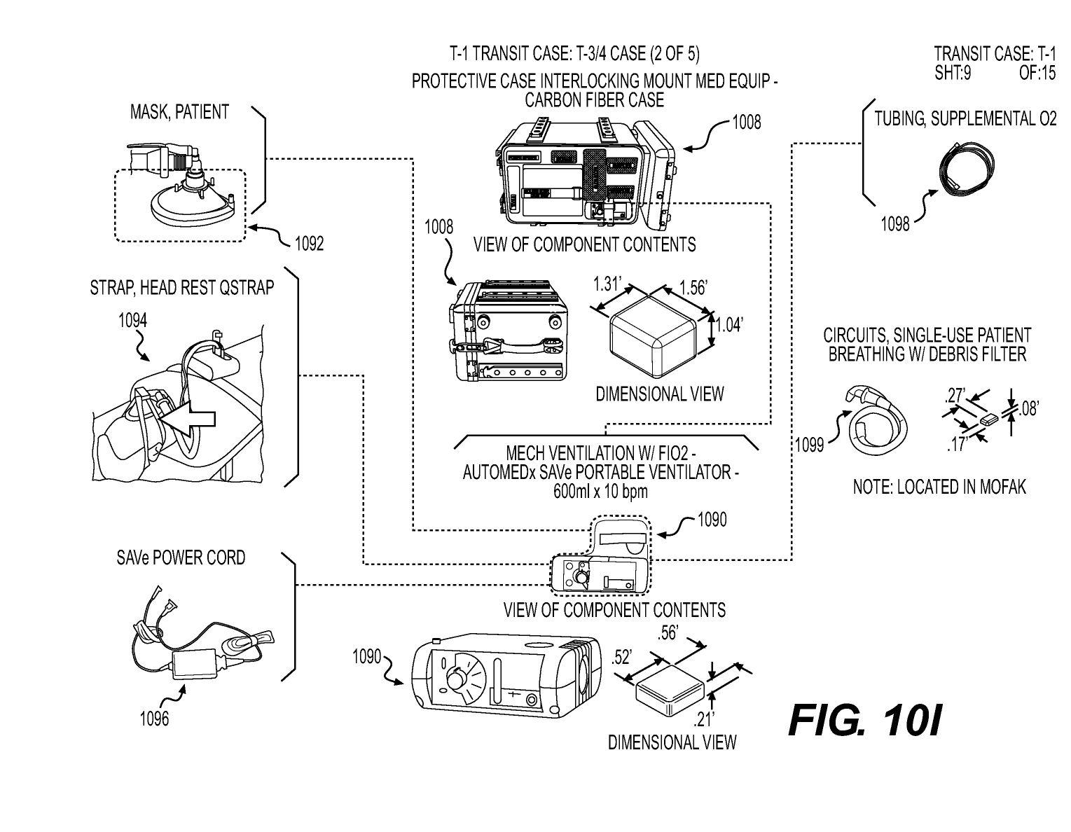

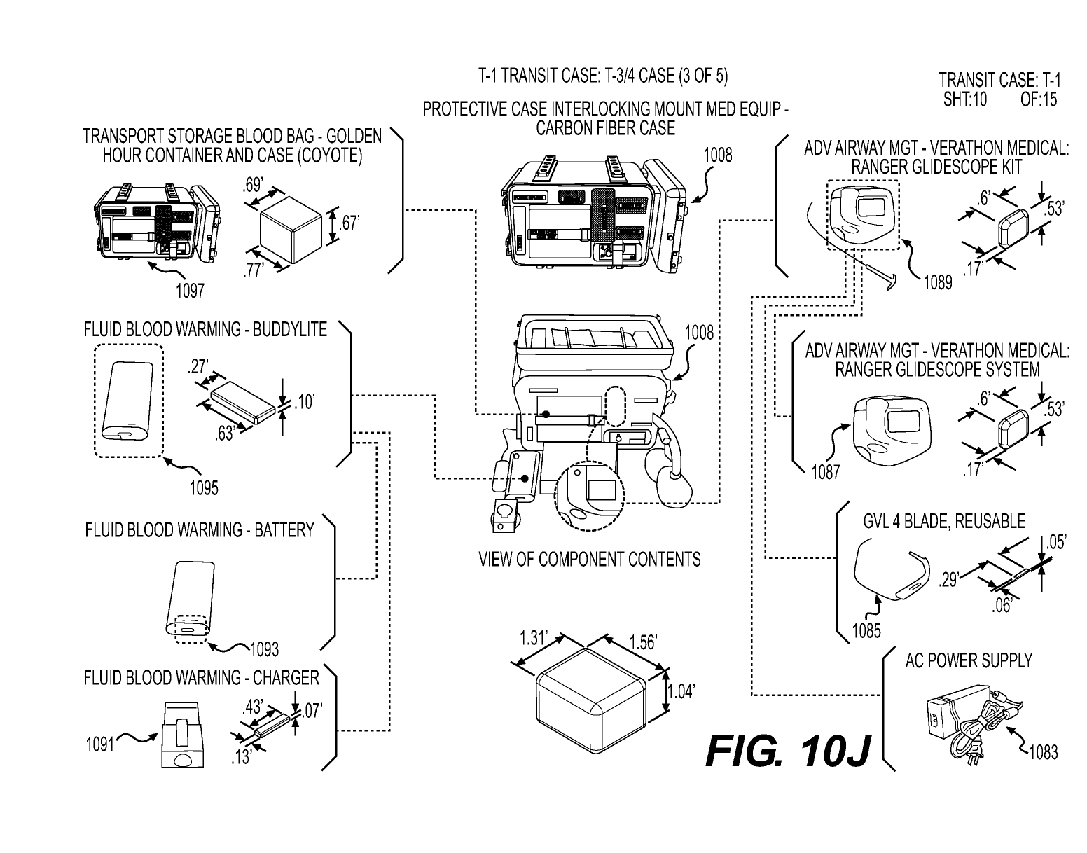

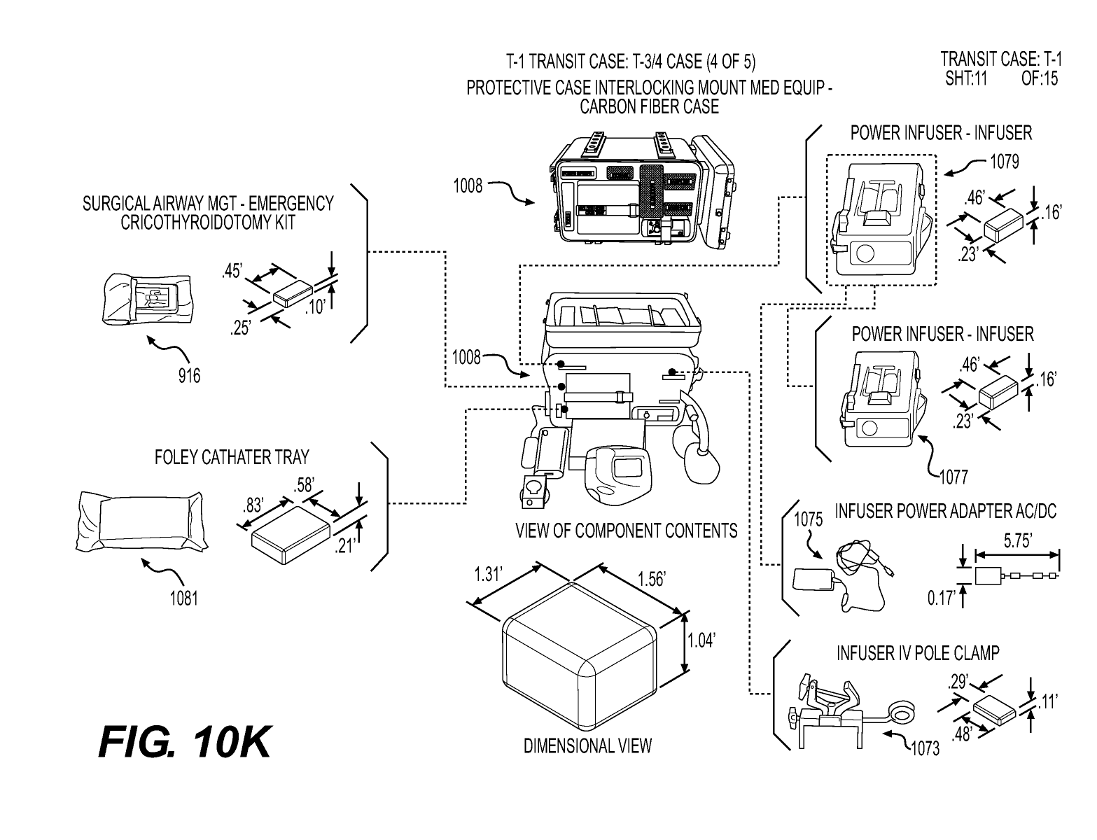

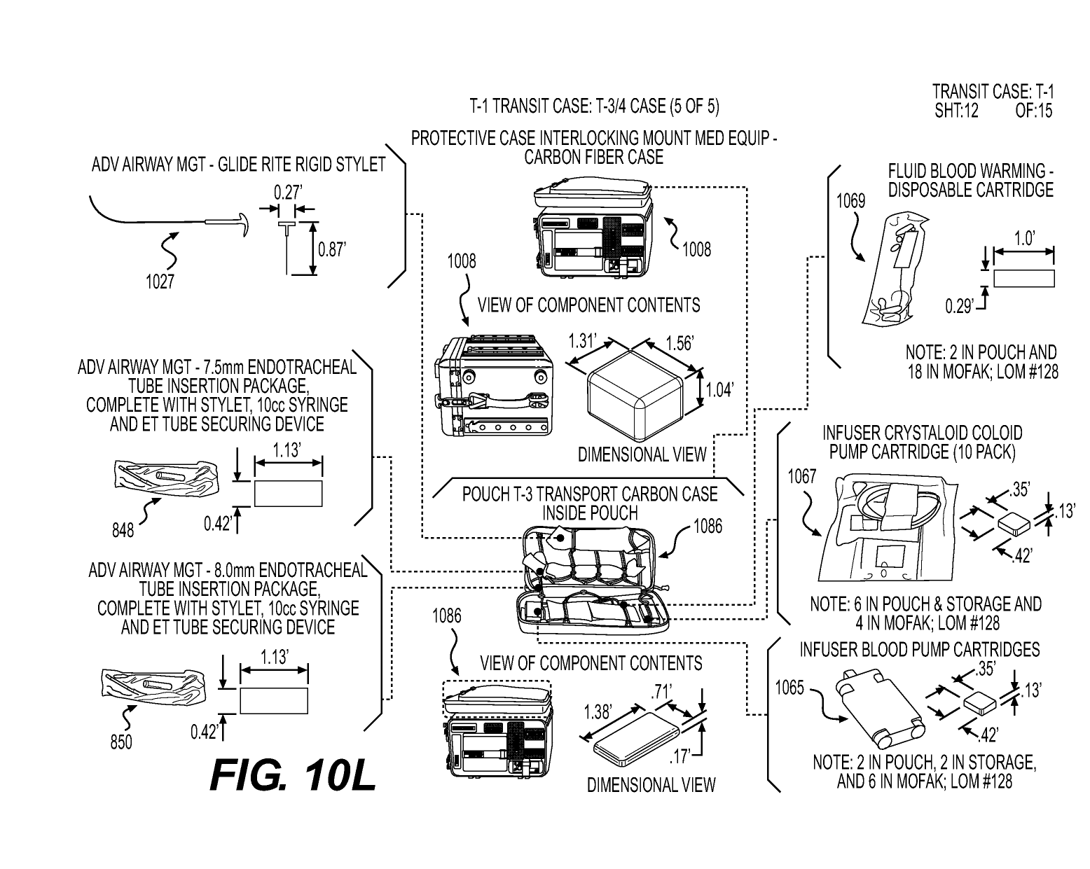

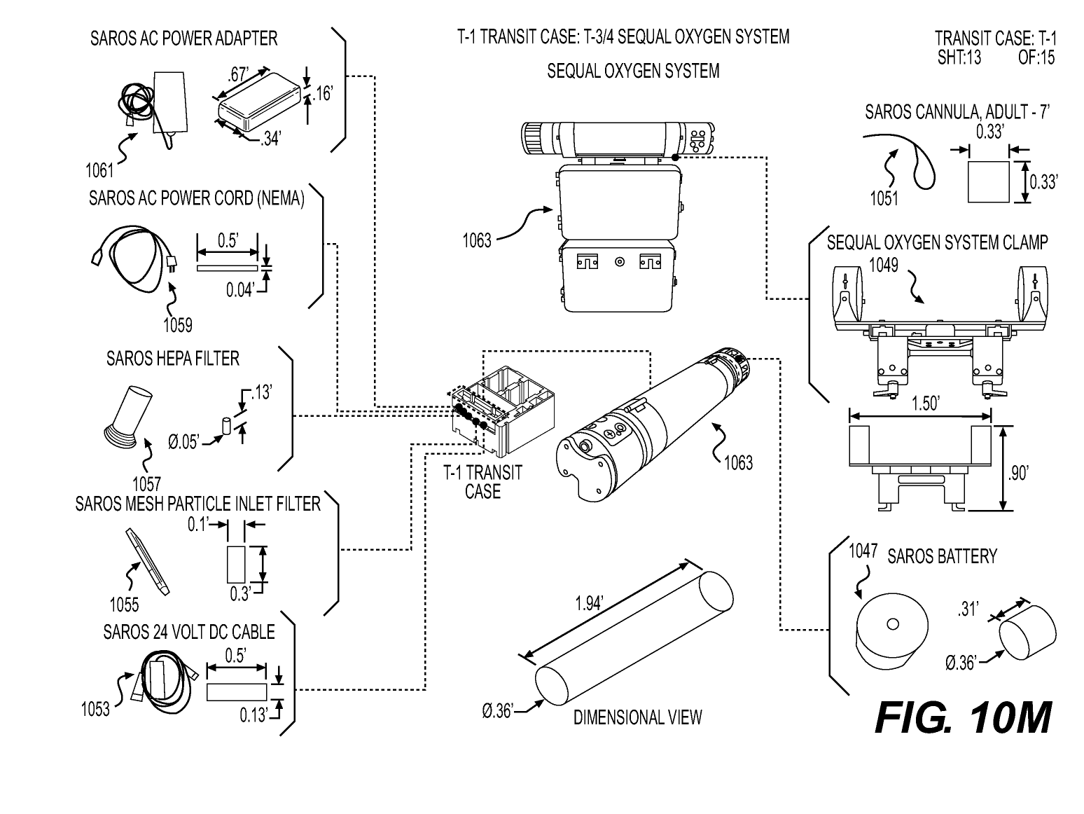

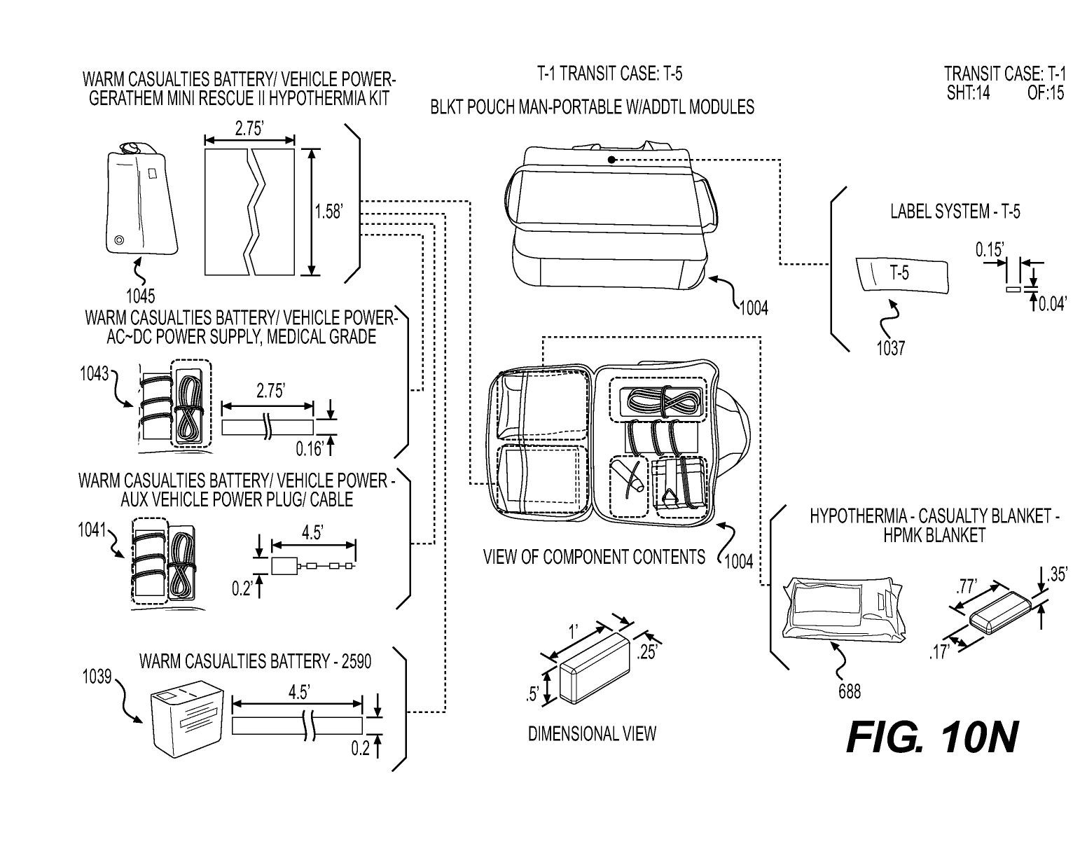

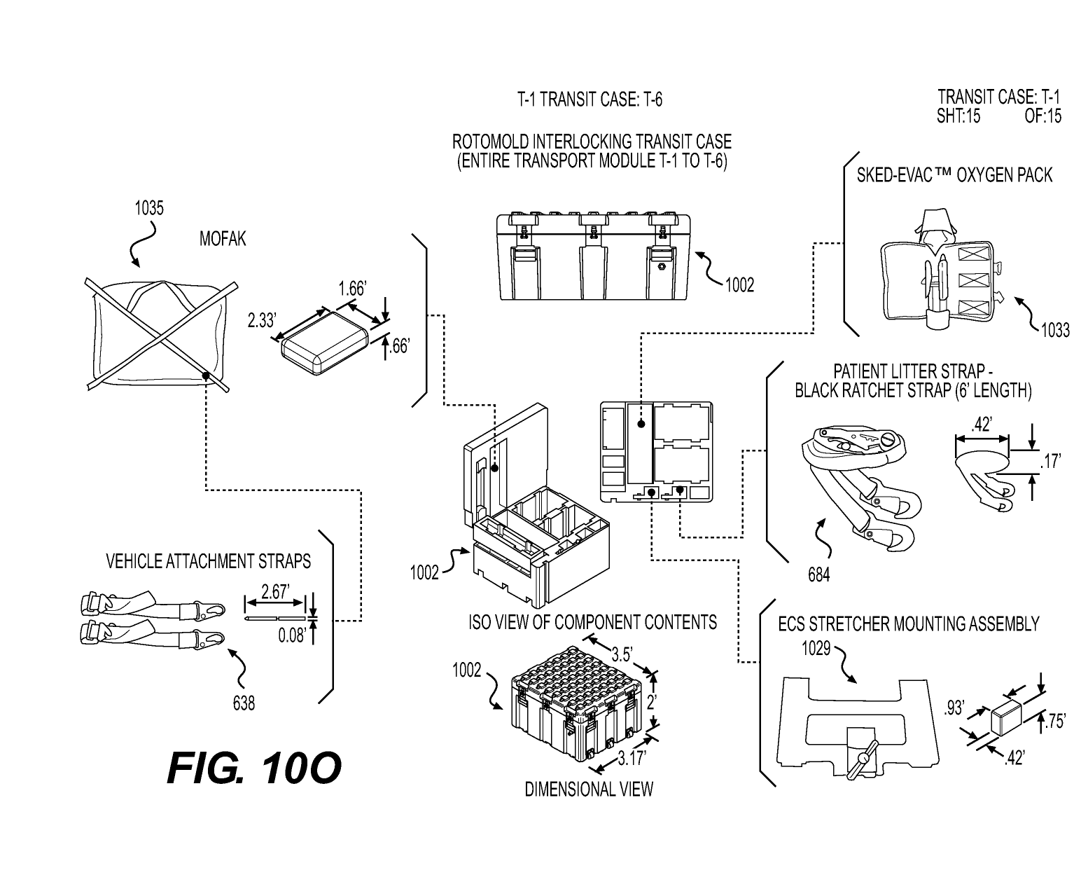

FIG. 10A illustrates the exemplary Case Contents of the T-1 transit case 128 of the transportation kit 106. Rotomold Interlocking Transit Case (Entire Transport Module T-1 to T-6) 1002 is shown containing Blanket Pouch Man-Portable with additional modules 1004, Repair Kit, Rotomold Interlocking Transit Case 568, Protective Case Interlocking Mount Med Equip—Carbon Fiber Case 1006, and Protective Case Interlocking Mount Med Equip—Carbon Fiber Case 1008. Rotomold Interlocking Transit Case (Entire Transport Module T-1 to T-6) 1002 may be NSN 8145-01-606-1544. Blkt Pouch Man-Portable with additional modules 1004 may be NSN 8465-01-607-0066, 0067, 0069 or 0071. Protective Case Interlocking Mount Med Equip—Carbon Fiber Case 1006 may be NSN 8145-01-606-9897, 9901, 9906 or 9929. Protective Case Interlocking Mount Med Equip—Carbon Fiber Case 1008 may be NSN 8145-01-606-9931, 9936, 9938 or 9944.

FIG. 10B illustrates the exemplary T-1 and T-2 Case (T-1/2) of the T-1 transit case 128. This is the first of six figures (FIGS. 10B-10G) showing the T-1/2 case. This case includes the T-1 and T-2 modules 130. Protective Case Interlocking Mount Med Equip—Carbon Fiber Case 1006 is shown containing Protective Case Interlocking Mount Med Equip—Carbon Fiber Case 1010, Protective Case Door Med Equip—Carbon Fiber door 1012, Cushion Pouch (Black Only) 1014 and Pouch T-1 Transport Carbon Fiber Case Pouch 1016. Pouch T-1 Transport Carbon Fiber Case Pouch 1016 is shown containing ACLS Protocol Meds—Epinephrine Enj USPO 1 mg per ml syringe—needle unit 10 ml 1018, ACLS Protocol Meds—Atropine Sulfate Inj USP 10ML Syringe with needle 1020 and ACLS Protocol Meds—Amiodarone HCL 50 mg/ml Inj, AMP, 3 ml 10 s 1022. Protective Case Interlocking Mount Med Equip—Carbon Fiber Case 1010 may be NSN 8145-01-606-1546, 1556, 1627 or 1632. Protective Case Door Med Equip—Carbon Fiber door 1012 may be NSN 8145-01-606-1584, 1589 or 1594. Pouch T-1 Transport Carbon Fiber Case Pouch 1016 may be NSN 8465-01-606-8663, 8666, 9945 or 9949. ACLS Protocol Meds—Epinephrine Enj USPO 1 mg per ml syringe—needle unit 10 ml 1018 may be NSN NDC 548331600. ACLS Protocol Meds—Amiodarone HCL 50 mg/ml Inj, AMP, 3 ml 10 s 1022 may be NSN NDC 67457015303.

FIG. 10C illustrates the exemplary T-1/2 Case of the T-1 transit case 128. This is the second of six figures (FIGS. 10B-10G) showing the T-1/2 case. Protective Case Interlocking Mount Med Equip—Carbon Fiber Case 1006 is shown containing Monitor Vitals, O2 Sat, Cardiac Output—Corp RAD 57 unit with SpO2, PR, PI, PVI, SpHb and SpOC 1024, Monitor Vitals, O2 Sat, Cardiac Output—Corp RAD 57 unit with SpO2, PR, PI, PVI, SpHb and SpOC 1026, Pleth Variability Index (PVI) (Software Upgrade) 1028 (Small patient diagnostic medical device), Rainbow DC-3 SC-360 Adult Reusable Spot Check Sensor, 360 tests, 3′, 1/box 1030, Label System—T-1 (PKG of 11) 1032, Thermometer Digital Clinical—RG Medical Data Therm II 1034, Disposable Probes—39″ Pack of 5 1036. Monitor Vitals, O2 Sat, Cardiac Output—Corp RAD 57 unit with SpO2, PR, PI, PVI, SpHb and SpOC 1024 may be NSN 6515-01-609-2004. Monitor Vitals, O2 Sat, Cardiac Output—Corp RAD 57 unit with SpO2, PR, PI, PVI, SpHb and SpOC 1026 may be NSN 6515-01-590-8493. Pleth Variability Index (PVI) (Software Upgrade) 1028? may be NSN 6515-01-590-8579. Rainbow DC-3 SC-360 Adult Reusable Spot Check Sensor, 360 tests, 3′, 1/box 1030 may be NSN 615-01-590-8899. Label System—T-1 (PKG of 11) 1032 may be NSN 7690-01-607-0029. Thermometer Digital Clinical—RG Medical Data Therm II 1034 may be NSN 6515-01-593-9284. Disposable Probes—39″ Pack of 5 1036 may be NSN 6515-01-595-5410.

FIG. 10D illustrates the exemplary T-1/2 Case of the T-1 transit case 128. This is the third of six figures (FIGS. 10B-10G) showing the T-1/2 case. Protective Case Interlocking Mount Med Equip—Carbon Fiber Case 1006 is shown containing Zoll AED Pro 1038, AED Pro Non-Rechargeable Lithium Battery Pack 1040, ECG Pads 1042, CPR-D Padz Airworthy 1044 and AED Pro ECG Cable AAMI 1046. Zoll AED Pro 1038 may be NSN 6515-01-609-1208. CPR-D Padz Airworthy 1044 may be NSN 6515-01-519-6131. AED Pro ECG Cable AAMI 1046 may be NSN 6515-01-530-8137.

FIG. 10E illustrates the exemplary T-1/2 Case of the T-1 transit case 128. This is the fourth of six figures (FIGS. 10B-10G) showing the T-1/2 case. Protective Case Interlocking Mount Med Equip—Carbon Fiber Case 1006 is shown containing Tempus Pro (TICS CASEVAC Configuration) 1071. Tempus Pro (TICS CASEVAC Configuration) 1071 is shown containing Tempus NIBP (Noninvasive blood pressure) Cuff—Adult 1048, Tempus Blood Pressure Hose 1050, Tempus Capnometer Cannula—Adult Nasal 1052, Tempus Capnometer Mainstream Airway Adapter 1054, Tempus Professional (Monitor) 1056 and Tempus SPO2 Cable (Pulse Oximeter Sensor) 1056. Tempus NIBP (Blood Pressure Cuff—Adult) 1048 may be NSN 6515-01-607-6997. Tempus Blood Pressure Hose 1050 may be NSN 6515-01-607-6986. Tempus SPO2 Cable (Pulse Oximeter Sensor) 1058 may be NSN 6515-01-607-6966.

FIG. 10F illustrates the exemplary T-1/2 Case of the T-1 transit case 128. This is the fifth of six figures (FIGS. 10B-10G) showing the T-1/2 case. Protective Case Interlocking Mount Med Equip—Carbon Fiber Case 1006 is shown containing Tempus Pro (TICS CASEVAC Configuration) 1071. Tempus Pro (TICS CASEVAC Configuration) 1071 is shown containing Tempus Mains Cable Pack 1060, Tempus Bluetooth Headset 1062, Tempus Pro 3-lead 4 Wire ECG Cable (AAMI) (with 4 ECG Pads) 1064, Tempus Elastic Wrist Straps (Pair) 1066, Tempus User Removable Battery, Rechargeable (Battery Pack Black) 1068 and Tempus Desktop Mains Power Supply 1070. Tempus Mains Cable Pack 1060 may be NSN6515-01-607-6951. Tempus Elastic Wrist Straps (Pair) 1066 may be NSN 6515-01-607-6929. Rechargeable (Battery Pack Black) 1068 may be NSN 6140-01-607-6918.

FIG. 10G illustrates the exemplary T-1/2 Case of the T-1 transit case 128. This is the sixth of six figures (FIGS. 10B-10G) showing the T-1/2 case. Protective Case Interlocking Mount Med Equip—Carbon Fiber Case 1006 is shown containing Tempus Pro (CASEVAC Configuration) 1071. Tempus Pro (CASEVAC Configuration) 1071 is shown containing Tempus Battery Charger 1072, Tempus User Removable Battery, Rechargeable Spare 1068, Tempus NIBP Blood Pressure Cuff—Large Adult 1074, Tempus NIBP Blood Pressure Cuff—Child 1076 and Tempus NIBP Blood Pressure Cuff—Thigh 1078. Tempus Battery Charger 1072 may be NSN 6130-01-607-9281. Tempus NIBP Blood Pressure Cuff—Large Adult 1074 may be NSN 6515-01-607-7480. Tempus NIBP Blood Pressure Cuff—Child 1076 may be NSN 6515-01-607-7499. Tempus NIBP Blood Pressure Cuff—Thigh 1078 may be NSN 6515-01-607-7487.

FIG. 10H illustrates the T-3 and T-4 (T-3/4) Case of the T-1 transit case 128. This is the first of five figures (FIGS. 10H-10L) showing the T-3/4 case. This case includes the T-3 and T-4 modules 134. Protective Case Interlocking Mount Med Equip—Carbon Fiber Case 1008 is shown containing Protective Case Interlocking Mount Med Equip—Carbon Fiber Case 1080, Protective Case Door Med Equip—Carbon Fiber Door 1082, Label System—T-3/4 (PKG of 12) 1084, Pouch T-3 Transport Carbon Case Inside Pouch 1086, Cushion Pouch (Black Only) 1088 and Mech Ventilation with FIO2—AutoMedx SAVe Portable Ventilator—600 mL×10 BPM 1090. Protective Case Interlocking Mount Med Equip—Carbon Fiber Case 1080 may be NSN 8145-01-606-1642, 1859, 1866 or 1871. Protective Case Door Med Equip—Carbon Fiber Door 1082 may be NSN 8145-01-606-1551, 1584, 1589 or 1594. Label System—T-3/4 (PKG of 12) 1084 may be NSN 7690-01-607-0043. Pouch T-3 Transport Carbon Case Inside Pouch 1086 may be NSN 8465-01-606-9965, 9967, 9969 or 0055. Cushion Pouch (Black Only) 1088 may be NSN 8135-01-606-1554. Mech Ventilation with FIO2—AutoMedx SAVe Portable Ventilator—600 mL×10 BPM 1090 may be NSN 6515-01-560-7288.

FIG. 10I illustrates the T-3/4 Case of the T-1 transit case 128. This is the second of five figures (FIGS. 10H-10L) showing the T-3/4 case. Protective Case Interlocking Mount Med Equip—Carbon Fiber Case 1008 is shown containing Mech Ventilation with FIO2—AutoMedx SAVe Portable Ventilator—600 mL×10 BPM 1090. Mech Ventilation with FIO2—AutoMedx SAVe Portable Ventilator—600 mL×10 BPM 1090 is shown containing Patient Mask 1092, Strap, Head RestQStrap 1094, SAVe Power Cord 1096, Tubing supplemental O2 1098 and Circuits, Single-Use Patient Breathing with Debris Filter 1099. Mask, Patient 1092 may be NSN 6515-01-580-3498. Strap, Head RestQStrap 1094 may be NSN 6515-01-580-2923. SAVe Power Cord 1096 may be NSN 6515-01-580-3522. Tubing, Supplemental O2 1098 may be NSN 6515-01-580-3532. Circuits, Single-Use Patient Breathing with Debris Filter 1099 may be NSN 6515-01-580-0768.

FIG. 10J illustrates the T-3/4 Case of the T-1 transit case 128. This is the third of five figures (FIGS. 10H-10L) showing the T-3/4 case. Protective Case Interlocking Mount Med Equip—Carbon Fiber Case 1008 is shown containing Transport Storage Blood Bag—Golden Hour Container and Case (Coyote) 1097, Fluid Blood Warming—BuddyLite 1095, Fluid Blood Warming—Battery 1093, Fluid Blood Warming—Charger 1091, Adv Airway Mgt—Verathon Medical: Ranger Glidescope Kit 1089, Adv Airway Mgt—Verathon Medical: Ranger Glidescope System 1087, GVL 4 Blade, Reusable 1085 and AC Power Supply 1083. Transport Storage Blood Bag—Golden Hour Container and Case (Coyote) 1097 may be NSN 6530-01-505-5306. Fluid Blood Warming—BuddyLite 1095 may be NSN 6515-01-598-8290. Fluid Blood Warming—Battery 1093 may be NSN 6140-01-598-3618. Fluid Blood Warming—Charger 1091 may be NSN 6130-01-598-8940. Adv Airway Mgt—Verathon Medical: Ranger Glidescope System 1087 may be NSN 6515-01-572-7262. GVL 4 Blade, Reusable 1085 may be NSN 6515-01-573-2460. AC Power Supply 1083 may be NSN 6515-01-573-2290.

FIG. 10K illustrates the T-3/4 Case of the T-1 transit case 128. This is the fourth of five figures (FIGS. 10H-10L) showing the T-3/4 case. Protective Case Interlocking Mount Med Equip—Carbon Fiber Case 1008 is shown containing Surgical Airway Mgt—Emergency Cricothyroidotomy Kit 916, Foley Catheter Tray 1081, Power Infuser—Infuser 1079, Power Fuser—Infuser 1077, Infuser Power Adapter AC/DC 1075 and Infuser IV Pole Clamp 1073. Power Fuser—Infuser 1077 may be NSN 6515-01-499-5112. Infuser Power Adapter AC/DC 1075 may be NSN 6515-01-466-1481. Infuser IV Pole Clamp 1073 may be NSN 6515-01-470-9907.

FIG. 10L illustrates the T-3/4 Case of the T-1 transit case 128. This is the fifth of five figures (FIGS. 10H-10L) showing the T-3/4 case. Protective Case Interlocking Mount Med Equip—Carbon Fiber Case 1008 is shown containing Adv Airway Mgt—Glide Rite Rigid Stylet 1027, Adv Airway Mgt—7.5 MM Endotracheal Tube insertion package, complete with stylet, 10 cc syringe and tube securing device 848, Adv Airway Mgt—8.0 MM Endotracheal Tube insertion package, complete with stylet, 10 cc syringe and tube securing device 850, Fluid Blood Warming—Disposable Cartridge 1069, Infuser Crystaloid Coloid Pump Cartridge (10 pack) 1067 and Infuser Blood Pump Cartridges 1065. Adv Airway Mgt—Glide Rite Rigid Stylet 1027 may be NSN 6515-01-588-8949. Fluid Blood Warming—Disposable Cartridge 1069 may be NSN 6515-01-542-4545. Infuser Crystaloid Coloid Pump Cartridge (10 pack) 1067 may be NSN 6515-01-466-1488. Infuser Blood Pump Cartridges 1065 may be NSN 6515-01-543-5684.

FIG. 10M illustrates the exemplary T-3/4 SeQual Oxygen System of the T-1 transit case 128. Sequal Oxygen System 1063 is shown containing Saros AC Power Adapter 1061, Saros AC Power Cord (NEMA) 1059, Saros HEPA Filter 1057, Saros Mesh Particle Inlet Filter 1055, Saros 24 Volt DC Cable 1053, Saros Cannula, Adult—7′ 1051, Sequal Oxygen System Camp 1049 and Saros Battery 1047. Sequal Oxygen System 1063 may be NSN 6515-01-588-3083. Saros AC Power Adapter 1061 may be NSN 6150-01-599-2024. Saros AC Power Cord (NEMA) 1059 may be NSN 6515-01-599-2017. Saros HEPA Filter 1057 may be NSN 6515-01-599-1970. Saros Mesh Particle Inlet Filter 1055 may be NSN 6515-01-599-2297. Saros 24 Volt DC Cable 1053 may be NSN 6150-01-599-2678. Saros Battery 1047 may be NSN 6140-01-599-3127.

FIG. 10N illustrates the exemplary T-5 module 136 of the T-1 transit case 128. Blanket Pouch Man-Portable with additional modules 1004 is shown containing Warm Casualties Battery/Vehicle Power—Geratherm Mini Rescue II Hypothermia kit 1045, Warm Casualties Battery/Vehicle Power—AC-DC Power Supply, Medical Grade 1043, Warm Casualties Battery/Vehicle Power—Aux Vehicle Power Plug/Cable 1041, Warm Casualties Battery—2590 1039, Label System—T-5 1037 and Hypothermia—Casualty Blanket—HPMK Blanket 688. Warm Casualties Battery/Vehicle Power—Geratherm Mini Rescue II Hypothermia kit 1045 may be NSN 6532-01-596-1253. Warm Casualties Battery/Vehicle Power—Aux Vehicle Power Plug/Cable 1041 may be NSN 6150-01-607-0154. Warm Casualties Battery—2590 1039 may be NSN 6140-01-533-3527. Label System—T-5 1037 may be NSN 7690-01-607-0051.

FIG. 10O illustrates the exemplary T-6 module 138 of the T-1 transit case 128. Rotomold Interlocking Transit Case (Entire Transport Module T-1 to T-6) 1002 is shown containing MOFAK 1035, Vehicle Attachment Straps 638, SKED-EVAC (trademark) Oxygen Pack 1033, Patient Litter Strap—Black Ratchet Strap (6′ Length) 684 and ECS Stretcher Mounting Assembly 1029. MOFAK 1035 may be NSN 8465-01-607-0156, 0157, 0159 or 0165. SKED-EVAC (trademark) Oxygen Pack 1033 may be NSN 6530-01-537-8511. ECS Stretcher Mounting Assembly 1029 may be NSN 5340-01-606-2195.

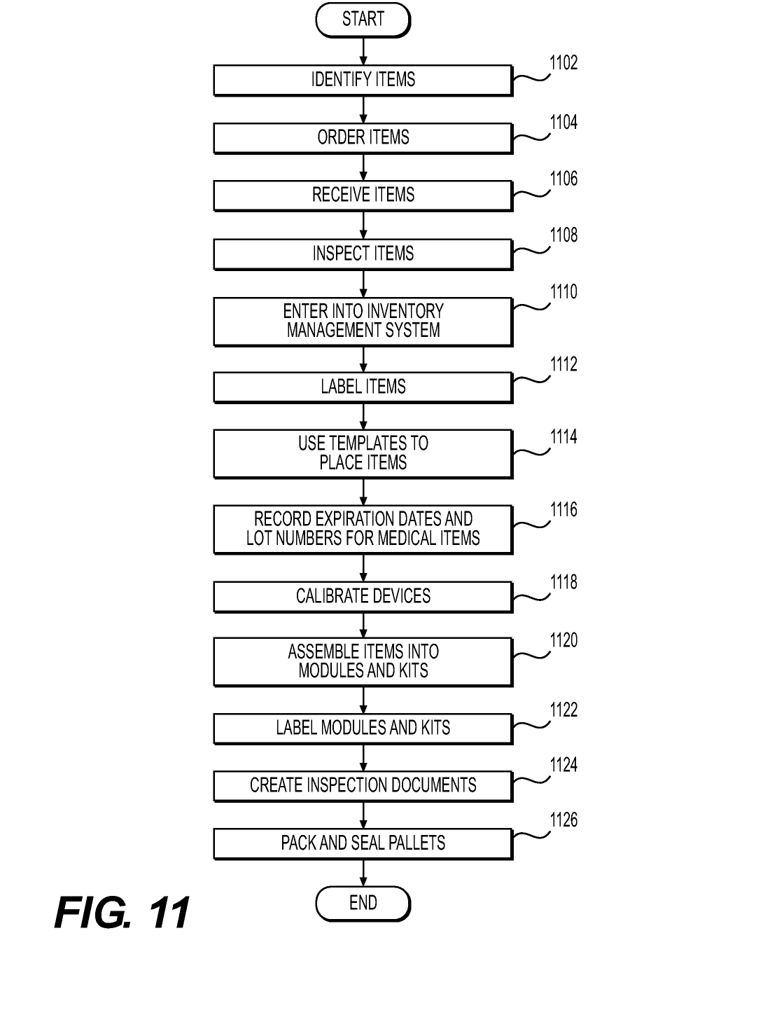

FIG. 11 is a flowchart that illustrates an exemplary method for providing medical kits in accordance with the present invention. An exemplary process for creating the kits is described below, however, other processes may be used, according to the components and configurations of FIGS. 1-10O, a user or company may procure, pack and ship the components that make up the medical supply kits and set. This may be done, for example, in a warehouse, and this facility may be the place for kitting, distributing and servicing the kits. Components that make up the set may be supplied, for example, by vendors or sub-vendors. Kits may be shipped directly to any unit for time sensitive contingency deployments. The process may generally include identifying an item, ordering and receiving the item, and packing the item in the appropriate module or kit. The process may also include shipping and/or using the resulting kit.

In additional detail, a user may identify the items desired, according the set, kits and modules of FIGS. 1-10O (step 1102). The user may procure the items, for example, by ordering (step 1104) or otherwise procuring the items. The user may receive (step 1106) and inspect equipment (step 1108) for shipping damage, order accuracy and quantities. Incoming equipment may optionally be scanned and placed into an inventory management system, which may be a computer system for tracking the inventory (step 1110).

Furthermore, equipment for deliveries orders may be allocated to a specific location in the warehouse and labeled (step 1112). In one implementation, once the equipment is in for a delivery order, kits and modules are placed into an assembly. Wheeled kitting tables may be moved to the location of a delivery order in the warehouse to create kitting stations. Kitting templates that have pictures, part numbers, National Stock Numbers (NSN's) and the sequence of items are placed on the kitting tables to complete a kitting station. These custom kitting templates ensure accuracy to aid in the inspection process. These kitting templates may have part numbers, and product outlines that not only ensure products are correct in quantity but ensure that products are packed in the correct sequence to save medical providers time once they have opened a pouch.

Individual items are then placed on their respective templates (step 1114), and items and templates are inspected for accuracy and completeness. Then, expiration dates and lot numbers are recorded for medical items (step 1116). In one implementation, medical devices and other suitable components are calibrated (step 1118). Calibration ensures that medical devices are within tolerances before they are shipped to the end user for use on deployments. In one implementation, medical devices and items that are over $5,000 dollars, for example, are labeled with an Item Unique Identification (IUID) label after calibration.

Items are then assembled into modules and kits (step 1120). Modules and kits are labeled (step 1122) and, in one implementation, inspection documents are created for potential inspections (e.g., Defense Contract Management Agency (DCMA) inspections) (step 1124). After inspections, the set, kits, and modules are sealed with serialized self-locking tags, palletized, banded, and shrink-wrapped (step 1126). Pictures may be taken of the orders and inspections documents are scanned for DCMA. Shipments are scheduled for large orders on dedicated trucks, and the set and/or kits are delivered to the intended recipient (step 1128).

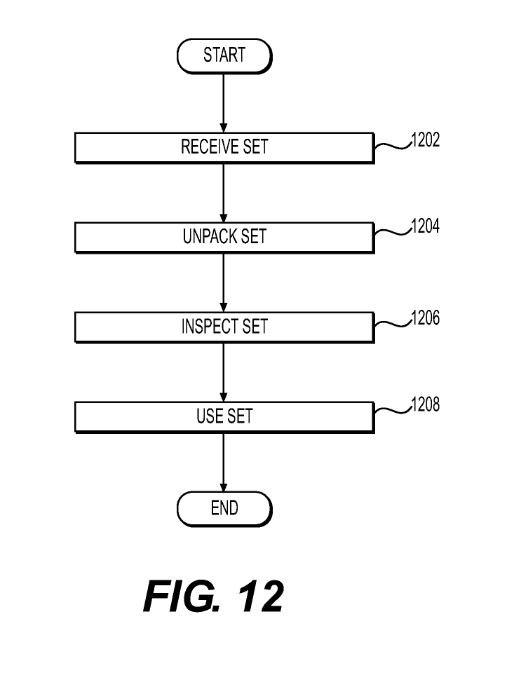

FIG. 12 illustrates an exemplary method for using medical kits in accordance with the present invention. An exemplary process for using the kits is described below, however, other processes may be used. The complete set is received (step 1202) and unpacked (step 1204). It may also be inspected (step 1206) and used (step 1208).

In one implementation, the complete set 100 contains 956 medical items that are comprised in the kits with “grab and go” pack configurations so they are mission ready. In one implementation, of these 956 items, 691 custom assemblies (contents of medical items placed in a pouch or pocket) are created that are packed in the same way in each single kit.

The foregoing description of various embodiments provides illustration and description, but is not intended to be exhaustive or to limit the invention to the precise form disclosed. Modifications and variations are possible in light of the above teachings or may be acquired from practice in accordance with the present invention.

APPENDIX

| | | | Transit | | | Total |
|---|---|---|---|---|---|---|
| FIG. | Kit | Module | Case | Sheet Title | Page | Page |
| 4A | Extraction | E-1 Patient Packaging | E-1/2 | Case Contents | 1 | 15 |
| 4B | Extraction | E-1 Patient Packaging | E-1/2 | E-1 Pack System | 2 | 15 |
| 4C | Extraction | E-1 Patient Packaging | E-1/2 | E-1 Top Pouch | 3 | 15 |
| 4D | Extraction | E-1 Patient Packaging | E-1/2 | E-1 Lateral Bag Set (1 of 2) | 4 | 15 |
| 4E | Extraction | E-1 Patient Packaging | E-1/2 | E-1 Lateral Bag Set (2 of 2) | 5 | 15 |
| 4F | Extraction | E-1 Patient Packaging | E-1/2 | E-1 Pack Contents (1 of 2) | 6 | 15 |
| 4G | Extraction | E-1 Patient Packaging | E-1/2 | E-1 Pack Contents (2 of 2) | 7 | 15 |
| 4H | Extraction | E-2 Rigging | E-1/2 | E-2 Pack System | 8 | 15 |
| 4I | Extraction | E-2 Rigging | E-1/2 | E-2 Top Pouch | 9 | 15 |
| 4J | Extraction | E-2 Rigging | E-1/2 | E-2 Bottom Pouch | 10 | 15 |
| 4K | Extraction | E-2 Rigging | E-1/2 | E-2 Pack Contents (1 of 2) | 11 | 15 |
| 4L | Extraction | E-2 Rigging | E-1/2 | E-2 Pack Contents (2 of 2) | 12 | 15 |
| 4M | Extraction | E-2 Rigging | E-1/2 | E-2 High-Angle Recovery Bag (1 of 2) | 13 | 15 |
| 4N | Extraction | E-2 Rigging | E-1/2 | E-2 High-Angle Recovery Bag (2 of 2) | 14 | 15 |
| 4O | Extraction | E-2 Rigging | E-1/2 | E-1 Bottom Storage | 15 | 15 |
| 5A | Extraction | E-3 Extrication | E-3 | Case Contents | 1 | 9 |
| 5B | Extraction | E-3 Extrication | E-3 | LE Pack System (1 of 2) | 2 | 9 |
| 5C | Extraction | E-3 Extrication | E-3 | LE Pack System (2 of 2) | 3 | 9 |
| 5D | Extraction | E-3 Extrication | E-3 | LE Pack System Pouches | 4 | 9 |
| 5E | Extraction | E-3 Extrication | E-3 | LE Pack Contents | 5 | 9 |
| 5F | Extraction | E-3 Extrication | E-3 | LL Pack System | 6 | 9 |
| 5G | Extraction | E-3 Extrication | E-3 | LL Pack System Pouches | 7 | 9 |
| 5H | Extraction | E-3 Extrication | E-3 | LL Pack Contents | 8 | 9 |
| 5I | Extraction | E-3 Extrication | E-3 | Bottom Storage | 9 | 9 |
| | | | Transit | | | Total |
| FIG. | Kit | Module | Case | Sheet Title | Page | Pages |
| 6A | Mobility | M-1 Tactical Field Care | M-1 | Case Contents | 1 | 10 |
| 6B | Mobility | M-2 Tactical Field Care | M-1 | M-2 Pack System (1 of 2) | 2 | 10 |
| 6C | Mobility | M-2 Tactical Field Care | M-1 | M-2 Pack System (2 of 2) | 3 | 10 |
| 6D | Mobility | M-2 Tactical Field Care | M-1 | M-2 Pouch Components | 4 | 10 |
| 6E | Mobility | M-2 Tactical Field Care | M-1 | M-2 IFAK Pouch (1 of 2) | 5 | 10 |
| 6F | Mobility | M-2 Tactical Field Care | M-1 | M-2 IFAK Pouch (2 of 2) | 6 | 10 |
| 6G | Mobility | M-2 Tactical Field Care | M-1 | M-2 Strao & Hvoothermia Pouches | 7 | 10 |
| 6H | Mobility—TFC | M-2 Tactical Field Care | M-1 | M-2 Mass Hem Pouch | 8 | 10 |
| 6I | Mobility—TFC | M-2 Tactical Field Care | M-1 | M-2 Circulation Pouch (1 of 2) | 9 | 10 |
| 6J | Mobility—TFC | M-2 Tactical Field Care | M-1 | M-2 Circulation Pouch (2 of 2) | 10 | 10 |
| 10A | Transportation - TT | T-1/2 ACLS | T-1 | Case Contents | 1 | 15 |
| 10B | Transoortation—TT | T-1/2 ACLS | T-1 | T-1/2 Case 11 of 6) | 2 | 15 |
| 10C | Transportation - TT | T-1/2 ACLS | T-1 | T-1/2 Case (2 of 6) | 3 | 15 |
| 10D | Transportation - TT | T-1/2 ACLS | T-1 | T-1/2 Case (3 of 6) | 4 | 15 |
| 10E | Transportation - TT | T-1/2 ACLS | T-1 | T-1/2 Case (4 of 6) | 5 | 15 |
| 10F | Transportation - TT | T-1/2 ACLS | T-1 | T-1/2 Case (5 of 6) | 6 | 15 |
| 10G | Transportation - TT | T-1/2 ACLS | T-1 | T-1/2 Case (6 of 6) | 7 | 15 |
| 10H | Transportation - TT | T-3/4 AAM/AFM | T-1 | T-3/4 Case (1 of 5) | 8 | 15 |
| 10I | Transportation - TT | T-3/4 AAM/AFM | T-1 | T-3/4 Case (2 of 5) | 9 | 15 |
| 10J | Transportation - TT | T-3/4 AAM/AFM | T-1 | T-3/4 Case (3 of 5) | 10 | 15 |
| 10K | Transportation - TT | T-3/4 AAM/AFM | T-1 | T-3/4 Case (4 of 5) | 11 | 15 |
| 10L | Transportation - TT | T-3/4 AAM/AFM | T-1 | T-3/4 Case (5 of 5) | 12 | 15 |
| 10M | Transportation - TT | T-3/4 AAM/AFM | T-1 | T-3/4 Sequal Oxygen System | 13 | 15 |
| 10N | Transportation - TT | T-5 Hypothermia | T-1 | T-5 | 14 | 15 |
| 10O | Transportation - TT | T-6 Universal Attach | T-1 | T-6 | 15 | 15 |
| 7 | Sustainment - TCS | S-1 Sustainment | S-1 Med | Med Storage Chest | 1 | 1 |
| | | | Transit | | | Total |
| Set | Kit | Module | Case | Sheet Title | Page | Pages |
| 8A | Sustainment - TCS | S-1 Sustainment | S-1 | Case Contents | 1 | 15 |
| 8B | Sustainment - TCS | S-1 Sustainment | S-1 | Pack System (1 of 2) | 2 | 15 |
| 8C | Sustainment - TCS | S-1 Sustainment | S-1 | Pack System (2 of 2) | 3 | 15 |
| 8D | Sustainment - TCS | S-1 Sustainment | S-1 | Top Pouch | 4 | 15 |
| V8E | Sustainment - TCS | S-1 Sustainment | S-1 | Bottom Pouch | 5 | 15 |
| 8F | Sustainment - TCS | S-1 Sustainment | S-1 | IFAK Pouch (1 of 2) | 6 | 15 |
| 8G | Sustainment - TCS | S-1 Sustainment | S-1 | IFAK Pouch (2 of 2) | 7 | 15 |
| 8H | Sustainment - TCS | S-1 Sustainment | S-1 | Airwav Pouch (1 of 2) | 8 | 15 |
| 8I | Sustainment - TCS | S-1 Sustainment | S-1 | Airway Pouch (2 of 2) | 9 | 15 |
| 8J | Sustainment - TCS | S-1 Sustainment | S-1 | Respiratory Pouch | 10 | 15 |
| 8K | Sustainment - TCS | S-1 Sustainment | S-1 | Circulation Pouch (1 of 2) | 11 | 15 |
| 8L | Sustainment - TCS | S-1 Sustainment | S-1 | Circulation Pouch (2 of 2) | 12 | 15 |
| 8M | Sustainment - TCS | S-1 Sustainment | S-1 | WWTK Pouch | 13 | 15 |
| 8N | Sustainment - TCS | S-1 Sustainment | S-1 | Hypothermia Pouch | 14 | 15 |
| 8O | Sustainment - TCS | S-1 Sustainment | S-1 | Bottom Storage | 15 | 15 |
| 8A | Sustainment - TCS | S-1 Sustainment | S-1 | Case Contents | 1 | 15 |
| 8B | Sustainment - TCS | S-1 Sustainment | S-1 | Pack System (1 of 2) | 2 | 15 |
| 8C | Sustainment - TCS | S-1 Sustainment | S-1 | Pack System (2 of 2) | 3 | 15 |
| 8D | Sustainment - TCS | S-1 Sustainment | S-1 | Top Pouch | 4 | 15 |
| 8E | Sustainment - TCS | S-1 Sustainment | S-1 | Bottom Pouch | 5 | 15 |
| 8F | Sustainment - TCS | S-1 Sustainment | S-1 | IFAK Pouch (1 of 2) | 6 | 15 |
| 8G | Sustainment - TCS | S-1 Sustainment | S-1 | IFAK Pouch (2 of 2) | 7 | 15 |
| 8H | Sustainment - TCS | S-1 Sustainment | S-1 | Airwav Pouch (1 of 2) | 8 | 15 |
| 8I | Sustainment - TCS | S-1 Sustainment | S-1 | Airway Pouch (2 of 2) | 9 | 15 |
| 8J | Sustainment - TCS | S-1 Sustainment | S-1 | Respiratory Pouch | 10 | 15 |
| 8K | Sustainment - TCS | S-1 Sustainment | S-1 | Circulation Pouch (1 of 2) | 11 | 15 |
| 8L | Sustainment - TCS | S-1 Sustainment | S-1 | Circulation Pouch (2 of 2) | 12 | 15 |
| 8M | Sustainment - TCS | S-1 Sustainment | S-1 | WWTK Pouch | 13 | 15 |
| 8N | Sustainment - TCS | S-1 Sustainment | S-1 | Hypothermia Pouch | 14 | 15 |
| 8O | Sustainment - TCS | S-1 Sustainment | S-1 | Bottom Storage | 15 | 15 |

1. A set of mission-ready tactical medical kits for military personnel, comprising:
an extraction medical kit comprising first equipment configured to assist a user in extracting a patient at a point of injury;
a mobility medical kit including second equipment configured to assist the user in moving the patient from a point of injury;
a transportation medical kit including third equipment configured to assist the user in treating and monitoring the patient during patient transport; and
a sustainment kit including fourth equipment configured to assist the user in sustaining a patient for up to three days, the fourth equipment including a pack system including two or more sustainment kit panels configured to attach pouches of medical equipment in the pack system;
wherein the extraction, mobility, transportation and sustainment medical kits are usable by the user without further assembly.

2. The set of tactical medical kits of claim 1, further comprising Rotomold transit cases containing at least a portion of each of the first, second, third and fourth equipment.

3. The set of tactical medical kits of claim 1, wherein:
the extraction medical kit comprises two Rotomold transit cases;
the mobility medical kit comprises a Rotomold transit case;
the transportation medical kit comprises a Rotomold transit case; and
the sustainment medical kit comprises two Rotomold transit cases.

4. The set of tactical medical kits of claim 3, wherein:
the extraction medical kit comprises four pack systems;
the mobility medical kit comprises a pack system;
the transportation medical kit comprises two carbon fiber cases; and
the sustainment medical kit comprises a pack system.

5. The set of mission-ready tactical medical kits of claim 1, wherein the mobility kit includes a folding rigid litter.

6. A set of mission-ready tactical medical kits for military personnel, comprising:
an extraction medical kit comprising first equipment configured to assist a user in extracting a patient at a point of injury;
a mobility medical kit including second equipment configured to assist the user in moving the patient from a point of injury;
a transportation medical kit including third equipment configured to assist the user in treating and monitoring the patient during patient transport; and
a sustainment kit including fourth equipment configured to assist the user in sustaining a patient for up to three days;
wherein the extraction, mobility, transportation and sustainment medical kits comprise Rotomold transit cases for storage of the first, second, third and fourth equipment.

7. The set of tactical medical kits of claim 6, wherein the extraction, mobility, transportation and sustainment medical kits are usable by the user without further assembly.

8. The set of tactical medical kits of claim 6, wherein the Rotomold transit cases are interlocking Rotomold transit cases.

9. The set of tactical medical kits of claim 6, wherein the extraction medical kit comprises a Rotomold transit case and two pack systems.

10. The set of tactical medical kits of claim 6, wherein the mobility kit includes a folding rigid litter.

12. The set of tactical medical kits of claim 6, wherein the transportation medical kit comprises a Rotomold transit case further comprising two carbon fiber cases.

13. The set of tactical medical kits of claim 6, wherein the sustainment medical kit comprises a Rotomold transit case and a pack system.

14. A method for providing a set of mission-ready tactical medical kits for military personnel, comprising:
receiving equipment to assemble medical kits;
assembling, from the received equipment, an extraction medical kit comprising first equipment configured to assist a user in extracting a patient at a point of injury;
assembling, from the received equipment, a mobility medical kit including second equipment configured to assist the user in moving the patient from a point of injury;
assembling, from the received equipment, a transportation medical kit including third equipment configured to assist the user in treating and monitoring the patient during patient transport; and
assembling, from the received equipment, a sustainment kit including fourth equipment configured to assist the user in sustaining a patient for up to three days, the fourth equipment including a pack system including two or more sustainment kit panels configured to attach pouches of medical equipment in the pack system;
wherein the extraction, mobility, transportation and sustainment medical kits are usable by the user without further assembly.

15. The method of claim 14, further comprising sending the set of tactical medical kits.

16. The method of claim 14, further comprising ordering the equipment used to assemble the extraction, mobility, transportation and sustainment medical kits.

17. The method of claim 14, wherein the received equipment further comprises Rotomold transit cases for containing at least a portion of each of the first, second, third and fourth equipment.

18. The method of claim 14, wherein:
the extraction medical kit comprises two Rotomold transit cases;
the mobility medical kit comprises a Rotomold transit case;
the transportation medical kit comprises a Rotomold transit case; and
the sustainment medical kit comprises two Rotomold transit cases.

19. A method for providing a set of mission-ready tactical medical kits for military personnel, comprising:
receiving equipment to assemble medical kits;
assembling, from the received equipment, an extraction medical kit comprising first equipment configured to assist a user in extracting a patient at a point of injury;
assembling, from the received equipment, a mobility medical kit including second equipment configured to assist the user in moving the patient from a point of injury;
assembling, from the received equipment, a transportation medical kit including third equipment configured to assist the user in treating and monitoring the patient during patient transport; and
assembling, from the received equipment a sustainment kit including fourth equipment configured to assist the user in sustaining a patient for up to three days;
wherein the extraction, mobility, transportation and sustainment medical kits comprise Rotomold transit cases for storage of the first, second, third and fourth equipment.

20. The method of claim 19, further comprising sending the set of tactical medical kits.

21. The method of claim 19, further comprising ordering the equipment used to assemble the extraction, mobility, transportation and sustainment medical kits.

22. The method of claim 19, wherein the received equipment further comprises Rotomold transit cases for containing at least a portion of each of the first, second, third and fourth equipment.

23. The method of claim 19, wherein:
the extraction medical kit comprises two Rotomold transit cases;
the mobility medical kit comprises a Rotomold transit case;
the transportation medical kit comprises a Rotomold transit case; and
the sustainment medical kit comprises two Rotomold transit cases.
